(19) United States
(12) Patent Application Publication
(10) Pub. No.: US 2026/0125366 A1
(43) Pub. Date: May 7, 2026

(54) METHODS FOR TREATING CANCER

(71) Applicant: SCORPION THERAPEUTICS, INC., Indianapolis, IN (US)

(72) Inventors: David ST. JEAN, JR., Natick, MA (US); Natasja BROOIJMANS, Boston, MA (US); Angel GUZMAN-PEREZ, Belmont, MA (US)

(21) Appl. No.: 19/118,135

(22) PCT Filed: Oct. 4, 2023
(86) PCT No.: PCT/US2023/075902

§ 371 Date: Apr. 3, 2025

Related U.S. Application Data
(60) Provisional application No. 63/414,173, filed on Oct. 7, 2022.

(51) Int. Cl. C07D 403/12; A61K 31/506; C07D 471/04

(52) U.S. Cl. CPC C07D 403/12; A61K 31/506; C07D 471/04

(57) ABSTRACT

This disclosure provides compounds of Formula (I), and pharmaceutically acceptable salts thereof, and compounds of Formula (II), and pharmaceutically acceptable salts thereof, that inhibit phosphatidylinositol 4,5-bisphosphate 3-kinase (PI3K) isoform alpha (PI3Kα). These chemical entities are useful, e.g., for treating a condition, disease or disorder in which increased (e.g., excessive) PI3Kα activation contributes to the pathology and/or symptoms and/or progression of the condition, disease or disorder (e.g., cancer) in a subject (e.g., a human). This disclosure also provides compositions containing the same as well as methods of using and making the same.

### CROSS REFERENCE TO RELATED APPLICATIONS

This application claims the benefit of U.S. Provisional Application No. 63/414,173, filed on Oct. 7, 2022, which is incorporated herein by reference in its entirety.

### SEQUENCE LISTING

This application contains a Sequence Listing that has been submitted electronically as an XML file named “50006-0102W01_ST26_SL.XML.” The XML file, created on Oct. 3, 2023, is 2,935 bytes in size. The material in the XML file is hereby incorporated by reference in its entirety.

### TECHNICAL FIELD

This disclosure provides compounds of Formula (I), and pharmaceutically acceptable salts thereof, and compounds of Formula (II), and pharmaceutically acceptable salts thereof, that inhibit phosphatidylinositol 4,5-bisphosphate 3-kinase (PI3K) isoform alpha (PI3Kα). These chemical entities are useful, e.g., for treating a condition, disease or disorder in which increased (e.g., excessive) PI3Kα activation contributes to the pathology and/or symptoms and/or progression of the condition, disease or disorder (e.g., cancer) in a subject (e.g., a human). This disclosure also provides compositions containing the same as well as methods of using and making the same.

### BACKGROUND

Phosphatidylinositol 4,5-bisphosphate 3-kinase (PI3K) isoform alpha (PI3Kα), encoded by the PIK3CA gene is a part of the PI3K/AKT/TOR signaling network and is altered in several human cancers. Several investigators have demonstrated the role of PI3K/AKT signaling is involved in physiological and pathophysiological functions that drive tumor progression such as metabolism, cell growth, proliferation, angiogenesis and metastasis. (See, Fruman, D. A. The PI3K Pathway in Human Disease. Cell 2017, 170, 605-635 and Janku, F. et al., Targeting the PI3K pathway in cancer: Are we making headway?Nat. Rev. Clin. Oncol.2018, 15, 273-291.) Suppression (e.g., pharmacological or genetic) of PI3K/AKT/TOR signaling may cause cancer cell death and regression of tumor growth.

The PI3K pathway can be activated via, for example, point mutation(s) of the PIK3CA gene or via inactivation of the phosphatase and tensin homolog (PTEN) gene. Activation of this pathway occurs in approximately 30-50% human cancers and contributes to resistance to various anti-cancer therapies. (See, Martini, M. et al., PI3K/AKT signaling pathway and cancer: An updated review. Ann. Med. 2014, 46, 372-383 and Bauer, T. M. et al., Targeting PI3 kinase in cancer. Pharmacol. Ther. 2015, 146, 53-60.) PI3K consists of three subunits: p85 regulatory subunit, p55 regulatory subunit, andp110 catalytic subunit. According to their different structures and specific substrates, PI3K is divided into 3 classes: classes I, II, and III. Class I PI3Ks include class IA and class IB PI3Ks. Class IA P13K, a heterodimer of p85 regulatory subunit and p110 catalytic subunit, is the type most clearly implicated in human cancer. Class IA PI3K includes p110α, p110β and p110δ catalytic subunits produced from different genes (PIK3CA, PIK3CB and PIK3CD, respectively), while p110γ produced by PIK3CG represents the only catalytic subunit in class IB PI3K. PIK3CA, the gene encoding the p110α subunit, is frequently mutated or amplified in many human cancers, such as breast cancer, colon cancer, gastric cancer, cervical cancer, prostate cancer, and lung cancer. (See, Samuels Y, et al. High frequency of mutations of the PIK3CA gene in human cancers. Science. 2004; 304:554.)

However, the development of PI3K inhibitors has been problematic for several reasons including (i) adaptive molecular mechanisms upon therapeutic inhibition of PI3K, (ii) inability to specifically inhibit signaling by PIK3CA mutations while sparing endogenous p110α, (iii) the limited use of these therapies in rational combinations, including those informed with strong mechanistic support, and (iv) dose-limiting toxicities that prevent sustained PI3K pathway suppression. (See, Hanker et al., Challenges for the Clinical Development of PI3K Inhibitors: strategies to Improve Their Impact in solid Tumors, Cancer Discovery, April 2019;9: 482-491.) For example, alpelisib is an alpha-selective PI3K inhibitor that is equipotent against wild-type and mutant forms of PI3Kα. However, the therapeutic benefit of alpelisib is limited by wild-type PI3Kα inhibition in normal tissues, resulting in dose-limiting toxicities including hyperglycemia.

Additionally, there are other factors and compensatory pathways derived from both clinical and in vitro lab studies, which affect PI3K signaling, such as HRAS and KRAS mutations, which reduce susceptibility to PI3K inhibitors (and knockdown of these has shown to improve sensitivity to PI3K inhibitors). (See, Misrha, R.; PI3K Inhibitors in Cancer: Clinical Implications and Adverse Effects. Int. J. Mol. Sci. 2021, 22, 3464.)

Domain deletions in PIK3CA can activate PI3K signaling significantly and also enhance the sensitivity to PI3K inhibitors. (See, Croessmann, S. et al., Clin. Cancer Res. 2018, 24, 1426-1435.) Thus, targeting PI3Kα represents an approach for the treatment of proliferative disorders such as cancer.

### SUMMARY

Some embodiments provide a compound of Formula (I):

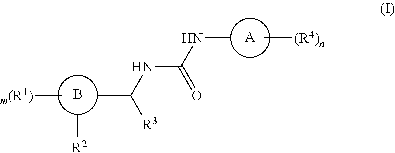

or a pharmaceutically acceptable salt thereof, wherein:
Ring B is a 9-membered heteroaryl group, wherein Ring B is not 2-benzofuranyl or 2-indolyl;
each R¹ is independently selected from halogen, hydroxyl, cyano, C1-C6 alkyl optionally substituted with hydroxyl, and C3-C6 cycloalkyl;
m is 0, 1, 2, or 3;
R² is halogen, hydroxyl, C1-C6 alkyl optionally substituted with hydroxyl, C1-C6 haloalkyl, or C3-C6 cycloalkyl optionally substituted with 1 or 2 fluoro;
R³ is a C1-C6 alkyl, a C1-C6 haloalkyl, or a C3-C6 cycloalkyl optionally substituted with 1 or 2 substituents independently selected from fluoro and C1-C6 alkyl;
Ring A is a 6-10 membered aryl, a C3-C8 cycloalkyl, a 5-10 membered heteroaryl, or a 4-10 membered heterocyclyl;
each R⁴ is independently selected from the group consisting of:
(i) halogen,
(ii) C1-C6 alkyl optionally substituted with 1 or 2 hydroxyl or —NR^{A}R^{B},
(iii) C1-C6 alkoxy optionally substituted with 1-2 substituents independently selected from hydroxyl and C3-C6 cycloalkyl,
(iv) C1-C6 haloalkyl,
(v) hydroxyl,
(vi) cyano,
(vii) —CO₂H,
(viii) —NR^{A}R^{B},
(ix) ═NR^{A2},
(x) —C(═O)NR^{C}R^{D},
(xi) —SO₂(NR^{E}R^{F}),
(xii) —SO₂(C1-C6 alkyl),
(xiii) —S(═O)(═NH)(C1-C6 alkyl),
(xiv) —C(═O)(C1-C6 alkyl),
(xv) —CO₂(C1-C6 alkyl),
(xvi) 5-6 membered heteroaryl optionally substituted with C1-C6 alkyl,
(xvii) 3-9 membered heterocyclyl optionally substituted with 1 or 2 independently selected R^{G}, and
(xviii) C3-C6 cycloalkyl optionally substituted with 1 or 2 independently selected R^{G};
n is 0, 1, or 2;
each R^{A}, R^{A1}, R^{B}, R^{B1}, R^{C}, R^{C1}, R^{D}, R^{D1}, R^{E}, and R^{F} is independently
(i) hydrogen,
(ii) hydroxyl,
(iii) 4-6 membered heterocyclyl,
(iv) C1-C6 haloalkyl,
(v) —C(═O)(C1-C6 alkyl),
(vi) —C(═O)O(C1-C6 alkyl),
(vii) —SO₂(C1-C6 alkyl),
(viii) C3-C6 cycloalkyl optionally substituted with hydroxyl, or
(ix) C1-C6 alkyl optionally substituted with 1-2 substituents independently selected from hydroxyl, —C(═O)NR^{B2}R^{C2}, 5-6 membered heteroaryl, C3-C6 cycloalkyl, —SO₂(C1-C6 alkyl), —CO₂H, and —SO₂(NH₂); or
R^{C} and R^{D}, together with the nitrogen atom to which they are attached form a 4-10 membered heterocyclyl optionally substituted with 1-2 substituents independently selected from hydroxyl, halogen, —C(═O)NR^{B1}R^{C1}, —SO₂(C1-C6 alkyl), —CO₂H, C1-C6 alkyl optionally substituted with hydroxyl, C1-C6 alkoxy, and C1-C6 haloalkoxy;
each R^{A2}, R^{B2}, and R^{C2} is independently hydrogen or C1-C6 alkyl;
each R^{G} is independently selected from the group consisting of: fluoro, cyano, hydroxyl, C1-C6 alkyl optionally substituted with hydroxyl, C1-C6 alkoxy, —NR^{A1}R^{B1}, ═NR^{A2}, —C(═O)NR^{C1}R^{D1}, —CO₂(C1-C6 alkyl), C1-C6 haloalkyl, C3-C6 cycloalkyl, C1-C6 haloalkoxy, —SO₂(C1-C6 alkyl), and —CO₂H.

Some embodiments provide a compound of Formula (II):

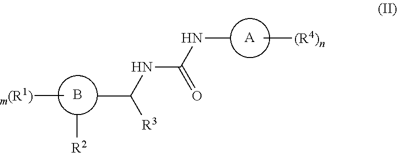

or a pharmaceutically acceptable salt thereof, wherein:
Ring B is a 9-membered heteroaryl group;
each R¹ is independently selected from halogen, hydroxyl, cyano, C1-C6 alkyl optionally substituted with hydroxyl, and C3-C6 cycloalkyl;
m is 0, 1, 2, or 3;
R² is halogen, hydroxyl, C1-C6 alkyl optionally substituted with hydroxyl, C1-C6 haloalkyl, or C3-C6 cycloalkyl optionally substituted with 1 or 2 fluoro;
R³ is a C1-C6 alkyl, a C1-C6 haloalkyl, or a C3-C6 cycloalkyl optionally substituted with 1 or 2 substituents independently selected from fluoro and C1-C6 alkyl;
Ring A is a 6-10 membered aryl, a C3-C8 cycloalkyl, a 5-10 membered heteroaryl, or a 4-10 membered heterocyclyl;
each R⁴ is independently selected from the group consisting of:
(i) halogen,
(ii) C1-C6 alkyl optionally substituted with 1 or 2 hydroxyl or —NR^{A}R^{B},
(iii) C1-C6 alkoxy optionally substituted with 1-2 substituents independently selected from hydroxyl and C3-C6 cycloalkyl,
(iv) C1-C6 haloalkyl,
(v) hydroxyl,
(vi) cyano,
(vii) —CO₂H,
(viii) —NR^{A}R^{B},
(ix) ═NR^{A2},
(x) —C(═O)NR^{C}R^{D},
(xi) —SO₂(NR^{E}R^{F}),
(xii) —SO₂(C1-C6 alkyl),
(xiii) —S(═O)(═NH)(C1-C6 alkyl),
(xiv) —C(═O)(C1-C6 alkyl),
(xv) —CO₂(C1-C6 alkyl),
(xvi) 5-6 membered heteroaryl optionally substituted with C1-C6 alkyl,
(xvii) 3-9 membered heterocyclyl optionally substituted with 1 or 2 independently selected R^{G}, and
(xviii) C3-C6 cycloalkyl optionally substituted with 1 or 2 independently selected R^{G};
n is 0, 1, or 2;
each R^{A}, R^{A1}, R^{B}, R^{B1}, R^{C}, R^{C1}, R^{D}, R^{D1}, R^{E}, and R^{F} is independently
(i) hydrogen,
(ii) hydroxyl,
(iii) 4-6 membered heterocyclyl,
(iv) C1-C6 haloalkyl,
(v) —C(═O)(C1-C6 alkyl),
(vi) —C(═O)O(C1-C6 alkyl),
(vii) —SO₂(C1-C6 alkyl),
(viii) C3-C6 cycloalkyl optionally substituted with hydroxyl, or
(ix) C1-C6 alkyl optionally substituted with 1-2 substituents independently selected from hydroxyl, —C(═O)NR^{B2}R^{C2}, 5-6 membered heteroaryl, C3-C6 cycloalkyl, —SO₂(C1-C6 alkyl), —CO₂H, and —SO₂(NH₂); or
R^{C} and R^{D}, together with the nitrogen atom to which they are attached form a 4-10 membered heterocyclyl optionally substituted with 1-2 substituents independently selected from hydroxyl, halogen, —C(═O)NR^{B1}R^{C1}, —SO₂(C1-C6 alkyl), —CO₂H, C1-C6 alkyl optionally substituted with hydroxyl, C1-C6 alkoxy, and C1-C6 haloalkoxy;
each R^{A2}, R^{B2}, and R^{C2} is independently hydrogen or C1-C6 alkyl;
   each R^{G} is independently selected from the group consisting of: fluoro, cyano, hydroxyl, C1-C6 alkyl optionally substituted with hydroxyl, C1-C6 alkoxy, —NR^{A1}R^{B1}, ═NR^{A2}, —C(═O)NR^{C1}R^{D1}, —CO₂(C1-C6 alkyl), C1-C6 haloalkyl, C3-C6 cycloalkyl, C1-C6 haloalkoxy, —SO₂(C1-C6 alkyl), and —CO₂H; and
wherein the compound of Formula (II) is not a compound disclosed in PCT/US2022/033255, which is hereby incorporated by reference in its entirety for the purpose of excluding the compounds contained therein.

Also provided herein is a pharmaceutical composition comprising a compound of Formula (I), or a pharmaceutically acceptable salt thereof, and one or more pharmaceutically acceptable excipients.

Provided herein is a method for treating cancer in a subject in need thereof, the method comprising administering to the subject a therapeutically effective amount of a compound of Formula (I), or a pharmaceutically acceptable salt thereof, or a pharmaceutical composition as provided herein.

Also provided herein is a method for treating cancer in a subject in need thereof, the method comprising (a) determining that the cancer is associated with a dysregulation of a PIK3CA gene, a PI3Kα protein, or expression or activity or level of any of the same; and (b) administering to the subject a therapeutically effective amount of a compound of Formula (I), or a pharmaceutically acceptable salt thereof, or a pharmaceutical composition as provided herein.

Provided herein is a method of treating a PI3Kα-associated disease or disorder in a subject, the method comprising administering to a subject identified or diagnosed as having a PI3Kα-associated disease or disorder a therapeutically effective amount of a compound of Formula (I), or a pharmaceutically acceptable salt thereof, or a pharmaceutical composition as provided herein.

This disclosure also provides a method of treating a PI3Kα-associated disease or disorder in a subject, the method comprising: determining that the cancer in the subject is a PI3Kα-associated disease or disorder; and administering to the subject a therapeutically effective amount of a compound of Formula (I), or a pharmaceutically acceptable salt thereof, or a pharmaceutical composition as provided herein.

Further provided herein is a method of treating a PI3Kα-associated cancer in a subject, the method comprising administering to a subject identified or diagnosed as having a PI3Kα-associated cancer a therapeutically effective amount of a compound of Formula (I), or a pharmaceutically acceptable salt thereof, or a pharmaceutical composition as provided herein.

This disclosure also provides a method of treating a PI3Kα-associated cancer in a subject, the method comprising: determining that the cancer in the subject is a PI3Kα-associated cancer; and administering to the subject a therapeutically effective amount of a compound of Formula (I), or a pharmaceutically acceptable salt thereof, or a pharmaceutical composition as provided herein.

Provided herein is a method of treating a subject, the method comprising administering a therapeutically effective amount of a compound of Formula (I), or a pharmaceutically acceptable salt thereof, or a pharmaceutical composition as provided herein, to a subject having a clinical record that indicates that the subject has a dysregulation of a PIK3CA gene, a PI3Kα protein, or expression or activity or level of any of the same.

This disclosure also provides a method for inhibiting PI3Kα in a mammalian cell, the method comprising contacting the mammalian cell with an effective amount of a compound of Formula (I), or a pharmaceutically acceptable salt thereof.

Also provided herein is a pharmaceutical composition comprising a compound of Formula (II), or a pharmaceutically acceptable salt thereof, and one or more pharmaceutically acceptable excipients.

Provided herein is a method for treating cancer in a subject in need thereof, the method comprising administering to the subject a therapeutically effective amount of a compound of Formula (II), or a pharmaceutically acceptable salt thereof, or a pharmaceutical composition as provided herein.

Also provided herein is a method for treating cancer in a subject in need thereof, the method comprising (a) determining that the cancer is associated with a dysregulation of a PIK3CA gene, a PI3Kα protein, or expression or activity or level of any of the same; and (b) administering to the subject a therapeutically effective amount of a compound of Formula (II), or a pharmaceutically acceptable salt thereof, or a pharmaceutical composition as provided herein.

Provided herein is a method of treating a PI3Kα-associated disease or disorder in a subject, the method comprising administering to a subject identified or diagnosed as having a PI3Kα-associated disease or disorder a therapeutically effective amount of a compound of Formula (II), or a pharmaceutically acceptable salt thereof, or a pharmaceutical composition as provided herein.

This disclosure also provides a method of treating a PI3Kα-associated disease or disorder in a subject, the method comprising: determining that the cancer in the subject is a PI3Kα-associated disease or disorder; and administering to the subject a therapeutically effective amount of a compound of Formula (II), or a pharmaceutically acceptable salt thereof, or a pharmaceutical composition as provided herein.

Further provided herein is a method of treating a PI3Kα-associated cancer in a subject, the method comprising administering to a subject identified or diagnosed as having a PI3Kα-associated cancer a therapeutically effective amount of a compound of Formula (II), or a pharmaceutically acceptable salt thereof, or a pharmaceutical composition as provided herein.

This disclosure also provides a method of treating a PI3Kα-associated cancer in a subject, the method comprising: determining that the cancer in the subject is a PI3Kα-associated cancer; and administering to the subject a therapeutically effective amount of a compound of Formula (II), or a pharmaceutically acceptable salt thereof, or a pharmaceutical composition as provided herein.

Provided herein is a method of treating a subject, the method comprising administering a therapeutically effective amount of a compound of Formula (II), or a pharmaceutically acceptable salt thereof, or a pharmaceutical composition as provided herein, to a subject having a clinical record that indicates that the subject has a dysregulation of a PIK3CA gene, a PI3Kα protein, or expression or activity or level of any of the same.

This disclosure also provides a method for inhibiting PI3Kα in a mammalian cell, the method comprising contacting the mammalian cell with an effective amount of a compound of Formula (II), or a pharmaceutically acceptable salt thereof.

Other embodiments include those described in the Detailed Description and/or in the claims.

### Additional Definitions

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

The term “about” when referring to a number or a numerical range means that the number or numerical range referred to is an approximation, for example, within experimental variability and/or statistical experimental error, and thus the number or numerical range may vary up to ±10% of the stated number or numerical range.

The term “acceptable” with respect to a formulation, composition or ingredient, as used herein, means having no persistent detrimental effect on the general health of the subject being treated.

The term “inhibit” or “inhibition of” means to reduce by a measurable amount, or to prevent entirely (e.g., 100% inhibition).

“API” refers to an active pharmaceutical ingredient.

The term “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutically acceptable salt” refers to a formulation of a compound that does not cause significant irritation to an organism to which it is administered and does not abrogate the biological activity and properties of the compound. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. In some instances, pharmaceutically acceptable salts are obtained by reacting a compound having acidic group described herein with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described hereinform with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid:organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “pharmaceutically acceptable excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: rectal, oral, intravenous, aerosol, parenteral, ophthalmic, pulmonary, and topical administration.

As used herein, the terms “subject,” “individual,” or “patient,” are used interchangeably, refers to any animal, including mammals such as primates (e.g., humans), mice, rats, other rodents, rabbits, dogs, cats, swine, cattle, sheep, horses, primates, and humans. In some embodiments, the subject is a human. In some embodiments, the subject has experienced and/or exhibited at least one symptom of the disease or disorder to be treated and/or prevented.

As used herein, terms “treat” or “treatment” refer to therapeutic or palliative measures. Beneficial or desired clinical results include, but are not limited to, alleviation, in whole or in part, of symptoms associated with a disease or disorder or condition, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

The term “halo” refers to fluoro (F), chloro (Cl), bromo (Br), or iodo (I).

The term “oxo” refers to a divalent doubly bonded oxygen atom (i.e., “=O”). As used herein, oxo groups are attached to carbon atoms to form carbonyls.

The term “hydroxyl” refers to an —OH radical.

The term “cyano” refers to a —CN radical.

The term “alkyl” refers to a saturated acyclic hydrocarbon radical that may be a straight chain or branched chain, containing the indicated number of carbon atoms. For example, C1io indicates that the group may have from 1 to 10 (inclusive) carbon atoms in it. Alkyl groups can either be unsubstituted or substituted with one or more substituents. Non-limiting examples include methyl, ethyl, iso-propyl, tert-butyl, n-hexyl. The term “saturated” as used in this context means only single bonds present between constituent carbon atoms and other available valences occupied by hydrogen and/or other substituents as defined herein.

The term “haloalkyl” refers to an alkyl, in which one or more hydrogen atoms is/are replaced with an independently selected halo.

The term “alkoxy” refers to an —O-alkyl radical (e.g., —OCH₃).

The term “aryl” refers to a 6-20 carbon mono-, bi-, tri- or polycyclic group wherein at least one ring in the system is aromatic (e.g., 6-carbon monocyclic, 10-carbon bicyclic, or 14-carbon tricyclic aromatic ring system); and wherein 0, 1, 2, 3, or 4 atoms of each ring may be substituted by a substituent. Examples of aryl groups include phenyl, naphthyl, tetrahydronaphthyl, and the like.

The term “cycloalkyl” as used herein refers to cyclic saturated hydrocarbon groups having, e.g., 3 to 20 ring carbons, preferably 3 to 16 ring carbons, and more preferably 3 to 12 ring carbons or 3-10 ring carbons or 3-6 ring carbons, wherein the cycloalkyl group may be optionally substituted. Examples of cycloalkyl groups include, without limitation, cyclopropyl, cyclobutyl, cyclopentyl, cyclohexyl, cycloheptyl, and cyclooctyl. Cycloalkyl may include multiple fused and/or bridged rings. Non-limiting examples of fused/bridged cycloalkyl includes: bicyclo[1.1.0]butane, bicyclo[2.1.0]pentane, bicyclo[1.1.1]pentane, bicyclo[3.1.0]hexane, bicyclo[2.1.1]hexane, bicyclo[3.2.0]heptane, bicyclo[4.1.0]heptane, bicyclo[2.2.1]heptane, bicyclo[3.1.1]heptane, bicyclo[4.2.0]octane, bicyclo[3.2.1]octane, bicyclo[2.2.2]octane, and the like. Cycloalkyl also includes spirocyclic rings (e.g., spirocyclic bicycle wherein two rings are connected through just one atom). Non-limiting examples of spirocyclic cycloalkyls include spiro[2.2]pentane, spiro[2.5]octane, spiro[3.5]nonane, spiro[3.5]nonane, spiro[3.5]nonane, spiro[4.4]nonane, spiro[2.6]nonane, spiro[4.5]decane, spiro[3.6]decane, spiro[5.5]undecane, and the like. The term “saturated” as used in this context means only single bonds present between constituent carbon atoms.

The term “heteroaryl”, as used herein, means a mono-, bi-, tri- or polycyclic group having 5 to 20 ring atoms, alternatively 5, 6, 9, 10, or 14 ring atoms; wherein at least one ring in the system contains one or more heteroatoms independently selected from the group consisting of N, O, and S and at least one ring in the system is aromatic (but does not have to be a ring which contains a heteroatom, e.g. tetrahydroisoquinolinyl, e.g., tetrahydroquinolinyl). Heteroaryl groups can either be unsubstituted or substituted with one or more substituents. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others. In some embodiments, the heteroaryl is selected from thienyl, pyridinyl, furyl, pyrazolyl, imidazolyl, isoindolinyl, pyranyl, pyrazinyl, and pyrimidinyl. For purposes of clarification, heteroaryl also includes aromatic lactams, aromatic cyclic ureas, or vinylogous analogs thereof, in which each ring nitrogen adjacent to a carbonyl is tertiary (i.e., all three valences are occupied by non-hydrogen substituents), such as one or more of pyridone

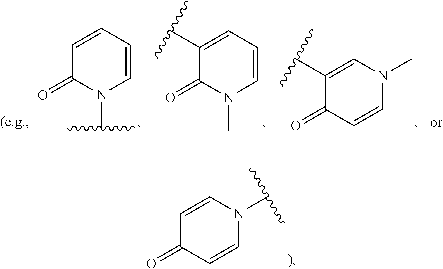

pyrimidone

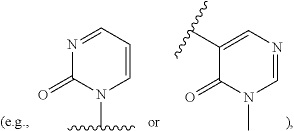

pyridazinone

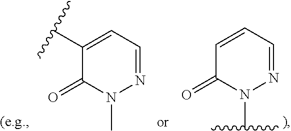

pyrazinone

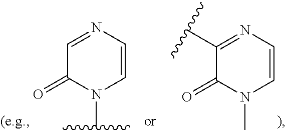

and imidazolone

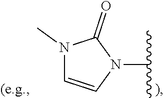 [SMILES: *n1ccn(C)c1=O]

wherein each ring nitrogen adjacent to a carbonyl is tertiary (i.e., the oxo group (i.e., “═O”) herein is a constituent part of the heteroaryl ring).

The term “heterocyclyl” refers to a mono-, bi-, tri-, or polycyclic saturated or partially unsaturated ring system with 3-16 ring atoms (e.g., 5-8 membered monocyclic, 8-12 membered bicyclic, or 11-14 membered tricyclic ring system) having 1-3 heteroatoms if monocyclic, 1-6 heteroatoms if bicyclic, or 1-9 heteroatoms if tricyclic or polycyclic, said heteroatoms selected from O, N, or S (e.g., carbon atoms and 1-3, 1-6, or 1-9 heteroatoms of N, O, or S if monocyclic, bicyclic, or tricyclic, respectively), wherein one or more ring atoms may be substituted by 1-3 oxo (forming, e.g., a lactam) and one or more N or S atoms may be substituted by 1-2 oxido (forming, e.g., an N-oxide, an S-oxide, or an S,S-dioxide), valence permitting; and wherein 0, 1, 2 or 3 atoms of each ring may be substituted by a substituent. Examples of heterocyclyl groups include piperazinyl, pyrrolidinyl, dioxanyl, morpholinyl, tetrahydrofuranyl, tetrahydropyridyl, dihydropyrazinyl, dihydropyridyl, dihydropyrrolyl, dihydrofuranyl, dihydrothiophenyl, and the like. Heterocyclyl may include multiple fused and bridged rings. Non-limiting examples of fused/bridged heteorocyclyl includes: 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, 3-azabicyclo[3.2.1]octane, 2-oxabicyclo[1.1.0]butane, 2-oxabicyclo[2.1.0]pentane, 2-oxabicyclo[1.1.1]pentane, 3-oxabicyclo[3.1.0]hexane, 5-oxabicyclo[2.1.1]hexane, 3-oxabicyclo[3.2.0]heptane, 3-oxabicyclo[4.1.0]heptane, 7-oxabicyclo[2.2.1]heptane, 6-oxabicyclo[3.1.1]heptane, 7-oxabicyclo[4.2.0]octane, 2-oxabicyclo[2.2.2]octane, 3-oxabicyclo[3.2.1]octane, and the like. Heterocyclyl also includes spirocyclic rings (e.g., spirocyclic bicycle wherein two rings are connected through just one atom). Non-limiting examples of spirocyclic heterocyclyls include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 7-azaspiro[4.5]decane 2,5-diazaspiro[3.6]decane, 3-azaspiro[5.5]undecane, 2-oxaspiro[2.2]pentane, 4-oxaspiro[2.5]octane, 1-oxaspiro[3.5]nonane, 2-oxaspiro[3.5]nonane, 7-oxaspiro[3.5]nonane, 2-oxaspiro[4.4]nonane, 6-oxaspiro[2.6]nonane, 1,7-dioxaspiro[4.5]decane, 2,5-dioxaspiro[3.6]decane, 1-oxaspiro[5.5]undecane, 3-oxaspiro[5.5]undecane, 3-oxa-9-azaspiro[5.5]undecane and the like.

As used herein, examples of aromatic rings include: benzene, pyridine, pyrimidine, pyrazine, pyridazine, pyridone, pyrrole, pyrazole, oxazole, thioazole, isoxazole, isothiazole, and the like.

As used herein, when a ring is described as being “partially unsaturated”, it means said ring has one or more additional degrees of unsaturation (in addition to the degree of unsaturation attributed to the ring itself; e.g., one or more double or tirple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

For the avoidance of doubt, and unless otherwise specified, for rings and cyclic groups (e.g., aryl, heteroaryl, heterocyclyl, cycloalkyl, and the like described herein) containing a sufficient number of ring atoms to form bicyclic or higher order ring systems (e.g., tricyclic, polycyclic ring systems), it is understood that such rings and cyclic groups encompass those having fused rings, including those in which the points of fusion are located (i) on adjacent ring atoms (e.g., [x.x.0] ring systems, in which 0 represents a zero atom bridge

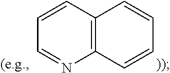

(ii) a single ring atom (spiro-fused ring systems)

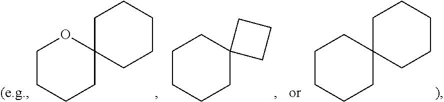

or (iii) a contiguous array of ring atoms (bridged ring systems having all bridge lengths >0)

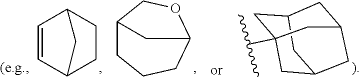

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, the compounds generically or specifically disclosed herein are intended to include all tautomeric forms. Thus, by way of example, a compound containing the moiety:

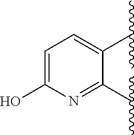 [SMILES: *c1ccc(O)nc1*]

encompasses the tautomeric form containing the moiety:

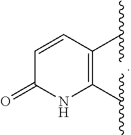 [SMILES: *c1ccc(=O)[nH]c1*]

Similarly, a pyridinyl or pyrimidinyl moiety that is described to be optionally substituted with hydroxyl encompasses pyridone or pyrimidone tautomeric forms.

The compounds provided herein may encompass various stereochemical forms. The compounds also encompass enantiomers (e.g., R and S isomers), diastereomers, as well as mixtures of enantiomers (e.g., R and S isomers) including racemic mixtures and mixtures of diastereomers, as well as individual enantiomers and diastereomers, which arise as a consequence of structural asymmetry in certain compounds. Unless otherwise indicated, when a disclosed compound is named or depicted by a structure without specifying the stereochemistry (e.g., a “flat” structure) and has one or more chiral centers, it is understood to represent all possible stereoisomers of the compound. Likewise, unless otherwise indicated, when a disclosed compound is named or depicted by a structure that specifies the stereochemistry (e.g., a structure with “wedge” and/or “dashed” bonds) and has one or more chiral centers, it is understood to represent the indicated stereoisomer of the compound.

The details of one or more embodiments of this disclosure are set forth in the accompanying drawings and the description below. Other features and advantages of the present disclosure will be apparent from the description and drawings, and from the claims.

### DETAILED DESCRIPTION

This disclosure provides compounds of Formula (I), and pharmaceutically acceptable salts thereof, that inhibit phosphatidylinositol 4,5-bisphosphate 3-kinase (PI3K) isoform alpha (PI3Kα). These chemical entities are useful, e.g., for treating a condition, disease or disorder in which increased (e.g., excessive) PI3Kα activation contributes to the pathology and/or symptoms and/or progression of the condition, disease or disorder (e.g., cancer) in a subject (e.g., a human). This disclosure also provides compositions containing the same as well as methods of using and making the same.

### Formulae (I) Compounds

Some embodiments provide a compound of Formula (I):

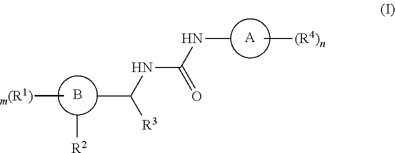

or a pharmaceutically acceptable salt thereof, wherein:
Ring B is a 9-membered heteroaryl group, wherein Ring B is not 2-benzofuranyl or 2-indolyl;
each R¹ is independently selected from halogen, hydroxyl, cyano, C1-C6 alkyl optionally substituted with hydroxyl, and C3-C6 cycloalkyl;
m is 0, 1, 2, or 3;
R² is halogen, hydroxyl, C1-C6 alkyl optionally substituted with hydroxyl, C1-C6 haloalkyl, or C3-C6 cycloalkyl optionally substituted with 1 or 2 fluoro;
R³ is a C1-C6 alkyl, a C1-C6 haloalkyl, or a C3-C6 cycloalkyl optionally substituted with 1 or 2 substituents independently selected from fluoro and C1-C6 alkyl;
Ring A is a 6-10 membered aryl, a C3-C8 cycloalkyl, a 5-10 membered heteroaryl, or a 4-10 membered heterocyclyl;
each R⁴ is independently selected from the group consisting of:
(i) halogen,
(ii) C1-C6 alkyl optionally substituted with 1 or 2 hydroxyl or —NR^{A}R^{B},
(iii) C1-C6 alkoxy optionally substituted with 1-2 substituents independently selected from hydroxyl and C3-C6 cycloalkyl,
(iv) C1-C6 haloalkyl,
(v) hydroxyl,
(vi) cyano,
(vii) —CO₂H,
(viii) —NR^{A}R^{B},
(ix) ═NR^{A2},
(x) —C(═O)NR^{C}R^{D},
(xi) —SO₂(NR^{E}R^{F}),
(xii) —SO₂(C1-C6 alkyl),
(xiii) —S(═O)(═NH)(C1-C6 alkyl),
(xiv) —C(═O)(C1-C6 alkyl),
(xv) —CO₂(C1-C6 alkyl),
(xvi) 5-6 membered heteroaryl optionally substituted with C1-C6 alkyl,
(xvii) 3-9 membered heterocyclyl optionally substituted with 1 or 2 independently selected R^{G}, and
(xviii) C3-C6 cycloalkyl optionally substituted with 1 or 2 independently selected R^{G};
n is 0, 1, or 2;
each R^{A}, R^{A1}, R^{B}, R^{B1}, R^{C}, R^{C1}, R^{D}, R^{D1}, R^{E}, and R^{F} is independently
(i) hydrogen,
(ii) hydroxyl,
(iii) 4-6 membered heterocyclyl,
(iv) C1-C6 haloalkyl,
(v) —C(═O)(C1-C6 alkyl),
(vi) —C(═O)O(C1-C6 alkyl),
(vii) —SO₂(C1-C6 alkyl),
(viii) C3-C6 cycloalkyl optionally substituted with hydroxyl, or
(ix) C1-C6 alkyl optionally substituted with 1-2 substituents independently selected from hydroxyl, —C(═O)NR^{B2}R^{C2}, 5-6 membered heteroaryl, C3-C6 cycloalkyl, —SO₂(C1-C6 alkyl), —CO₂H, and —SO₂(NH₂); or
R^{C} and R^{D}, together with the nitrogen atom to which they are attached form a 4-10 membered heterocyclyl optionally substituted with 1-2 substituents independently selected from hydroxyl, halogen, —C(═O)NR^{B1}R^{C1}, —SO₂(C1-C6 alkyl), —CO₂H, C1-C6 alkyl optionally substituted with hydroxyl, C1-C6 alkoxy, and C1-C6 haloalkoxy;
each R^{A2}, R^{B2}, and R^{C2} is independently hydrogen or C1-C6 alkyl;
each R^{G} is independently selected from the group consisting of: fluoro, cyano, hydroxyl, C1-C6 alkyl optionally substituted with hydroxyl, C1-C6 alkoxy, —NR^{A1}R^{B1}, ═NR^{A2}, —C(═O)NR^{C1}R^{D1}, —CO₂(C1-C6 alkyl), C1-C6 haloalkyl, C3-C6 cycloalkyl, C1-C6 haloalkoxy, —SO₂(C1-C6 alkyl), and —CO₂H.

In some embodiments, m is 0. In some embodiments, m is 1. In some embodiments, m is 2. In some embodiments, m is 3.

In some embodiments of Formula (I),

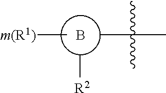

is selected from the group consisting of

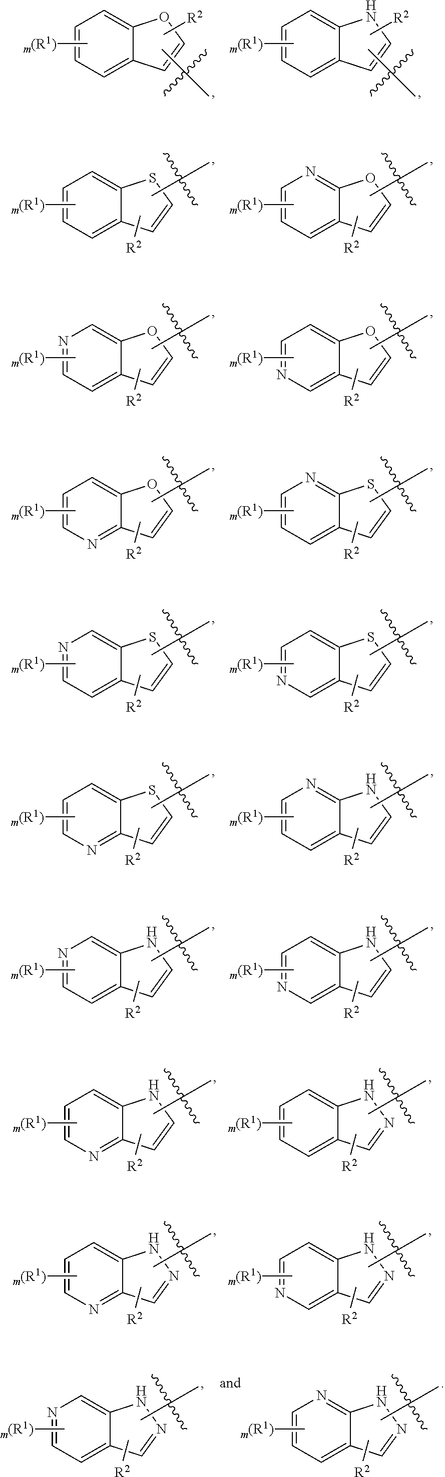

In some embodiments,

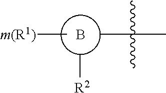

is selected from the group consisting of

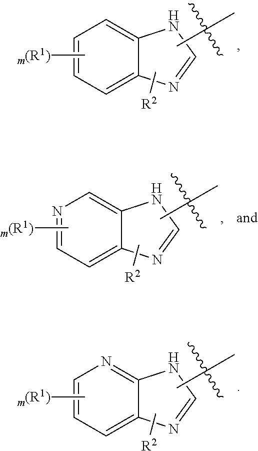

In some embodiments,

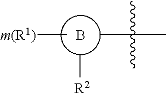

is selected from the group consisting of

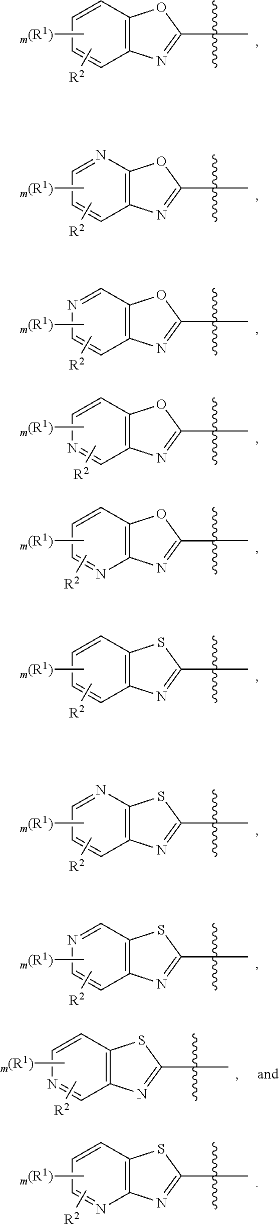

In some embodiments,

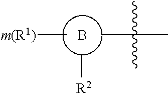

is selected from the group consisting of

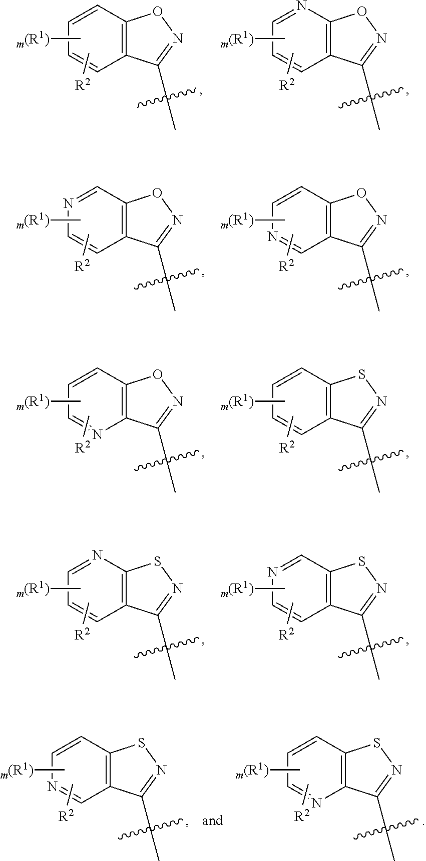

In some embodiments,

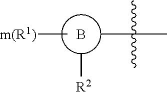

is selected from the group consisting of

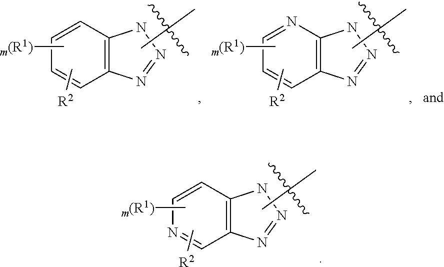

In some embodiments,

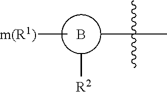

is selected from the group consisting of

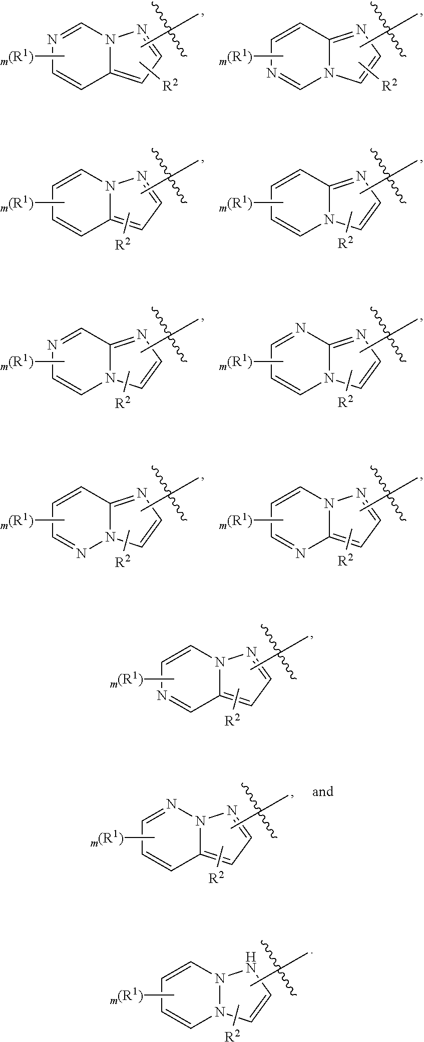

In some embodiments,

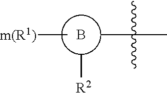

is selected from the group consisting of

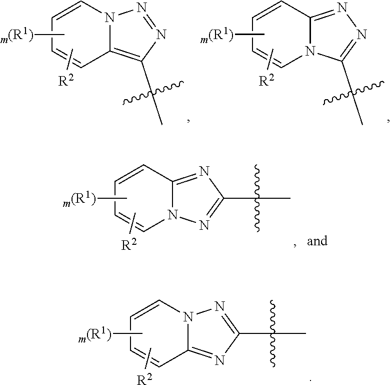

In some embodiments,

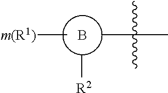

is selected from the group consisting of

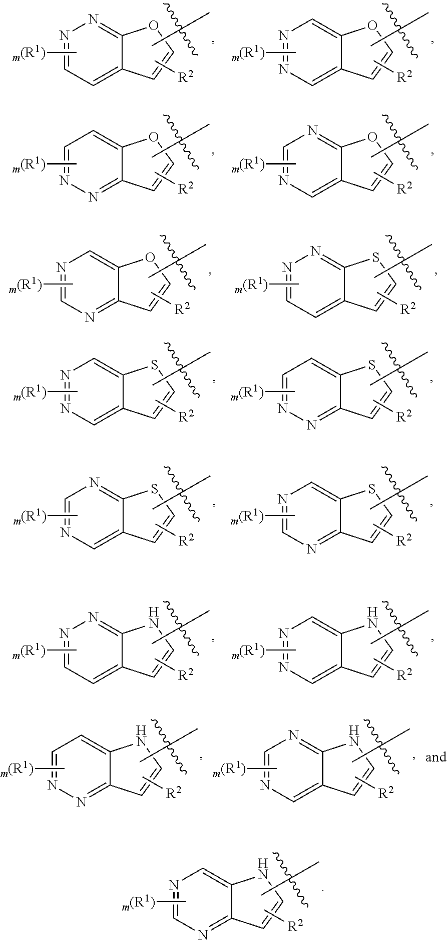

In some embodiments,

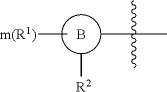

is selected from the group consisting of

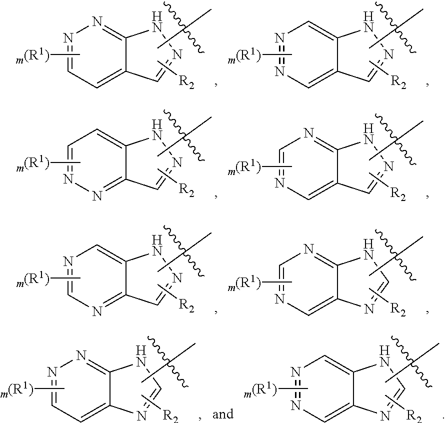

In some embodiments,

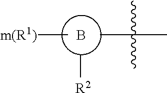

is selected from the group consisting of

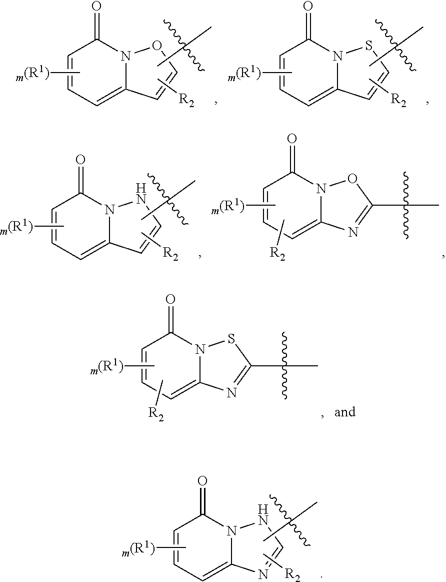

In some embodiments,

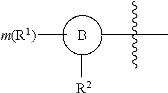

is selected from the group consisting of

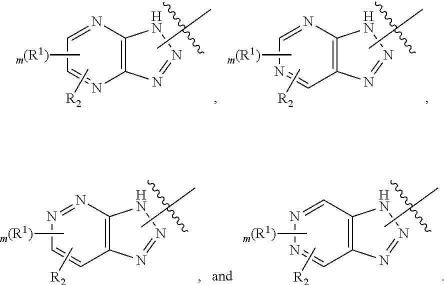

In some embodiments,

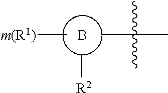

is selected from the group consisting of

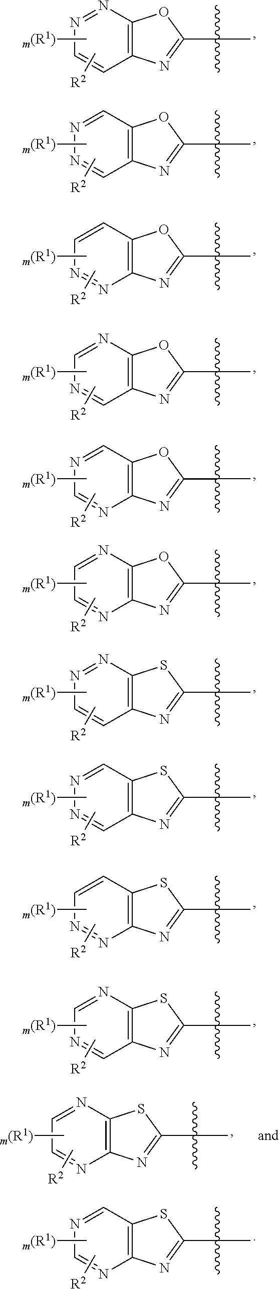

In some embodiments,

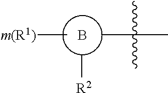

is selected from the group consisting of

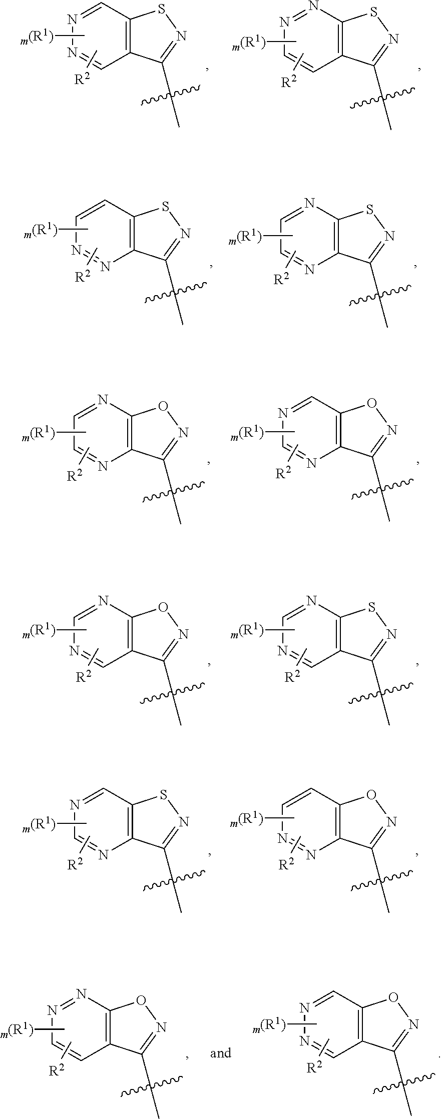

In some embodiments,

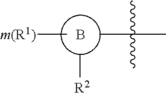

is selected from the group consisting of

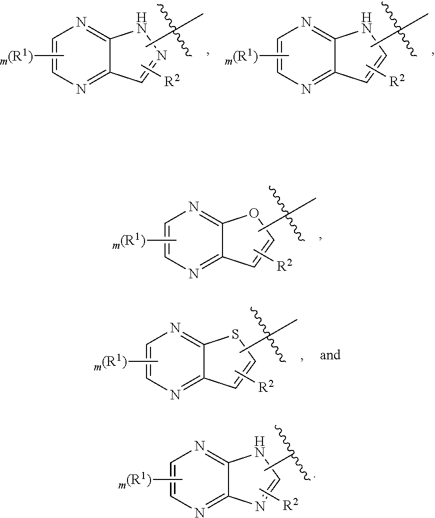

In some embodiments,

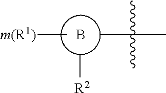

is selected from the group consisting of

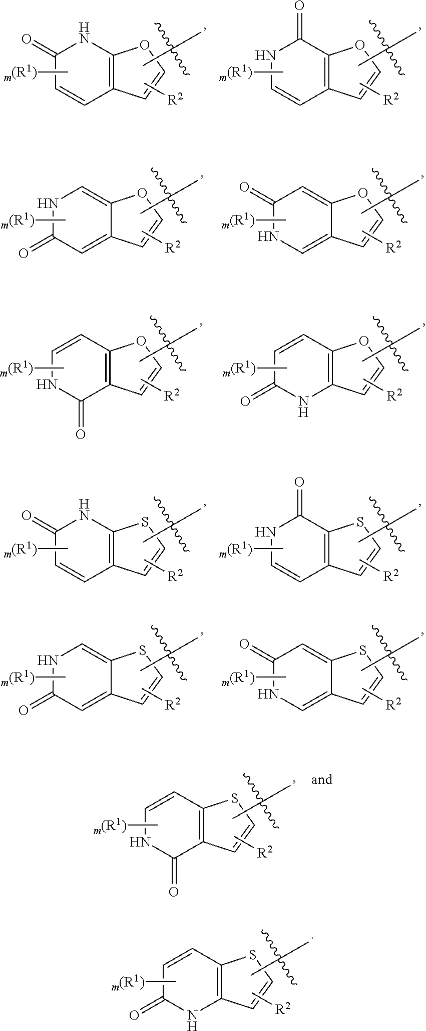

In some embodiments,

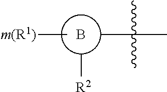

is selected from the group consisting of

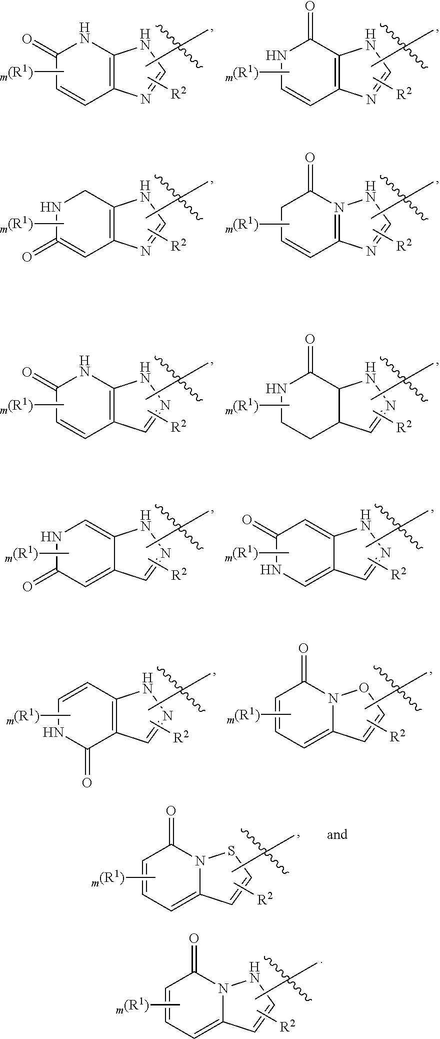

In some embodiments,

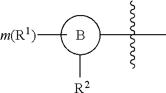

is selected from the group consisting of

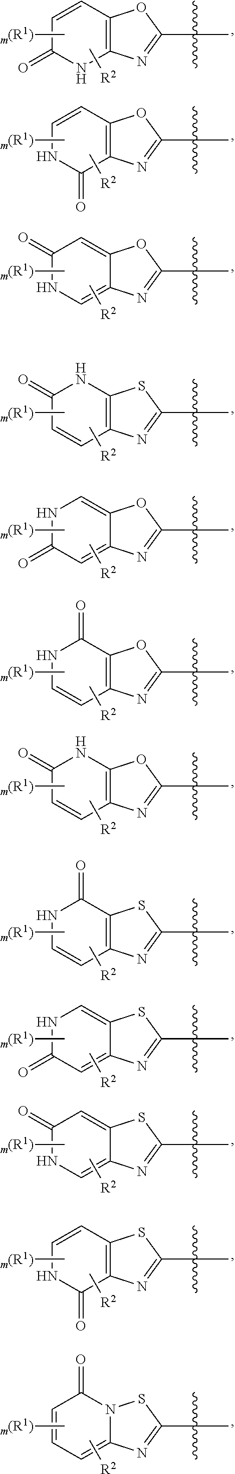

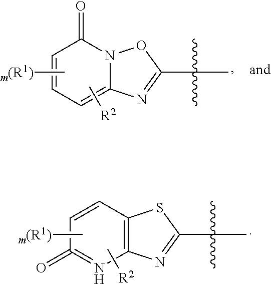

In some embodiments,

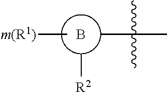

is selected from the group consisting of

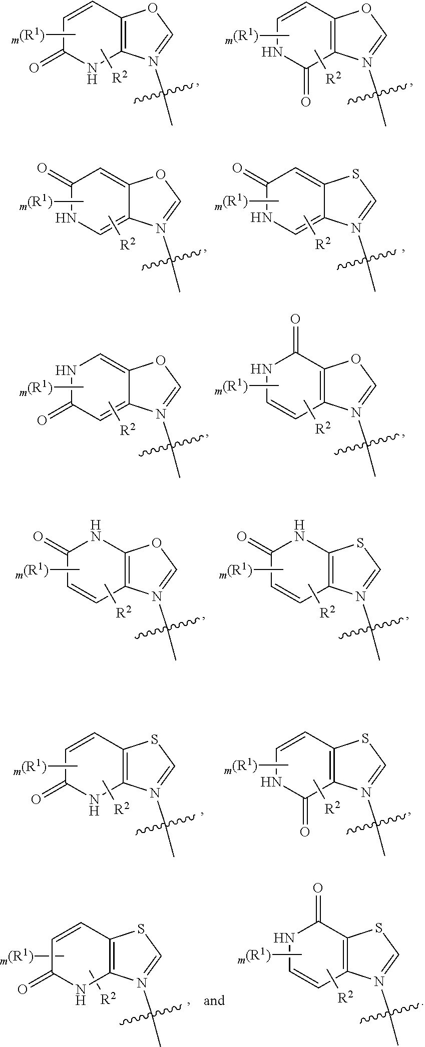

In some embodiments,

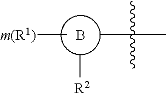

is selected from the group consisting of

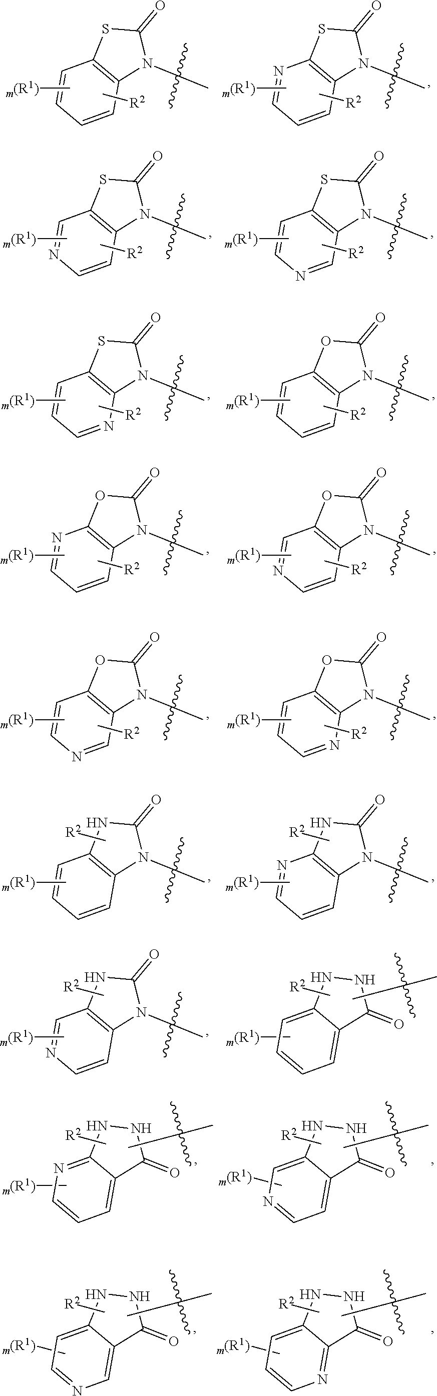

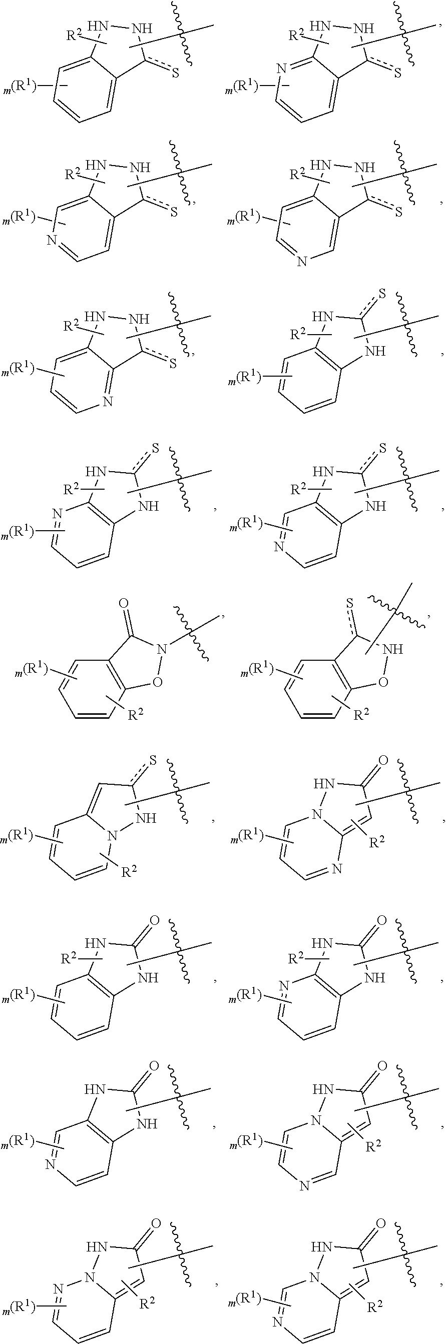

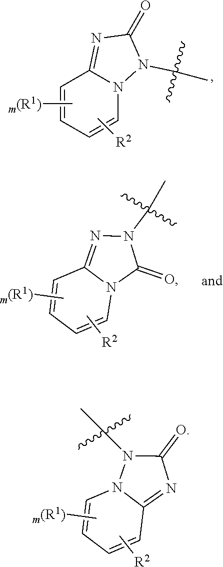

In some embodiments,

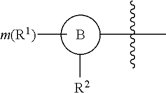

is selected from the group consisting of

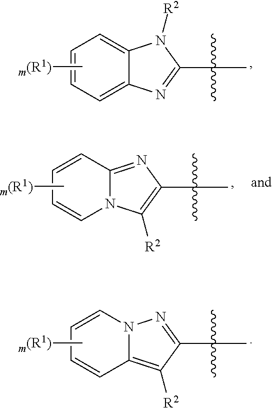

In some embodiments,

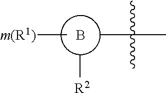

is

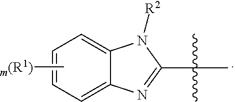

In some embodiments,

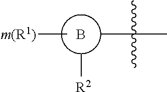

is selected from the group consisting of

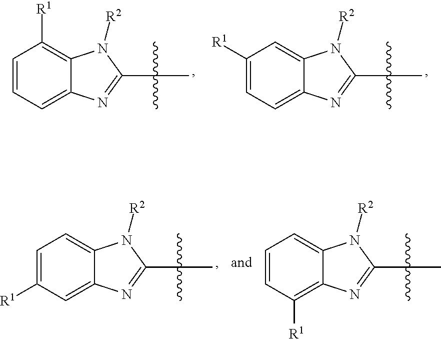

In some embodiments,

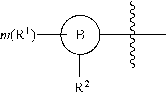

is selected from the group consisting of

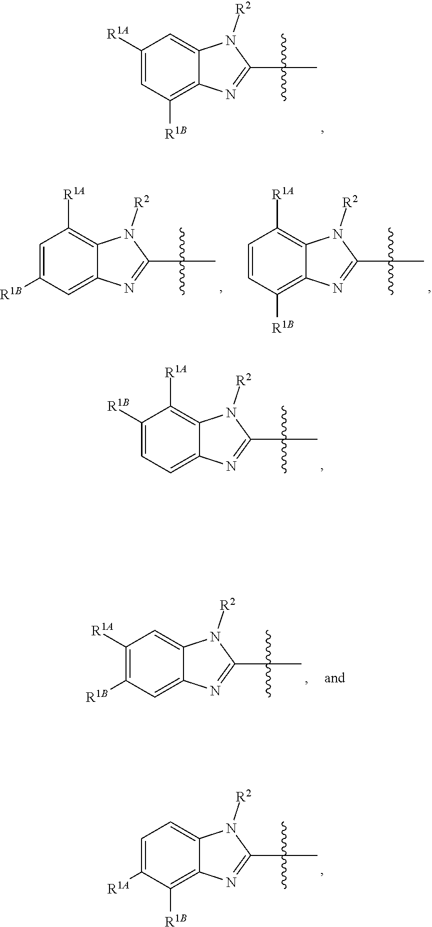

wherein R^{1A} and R^{1B} are independently selected from R¹.

In some embodiments,

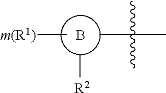

is

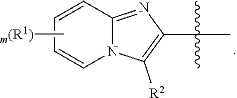

In some embodiments,

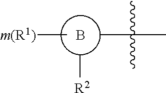

is selected from the group consisting of

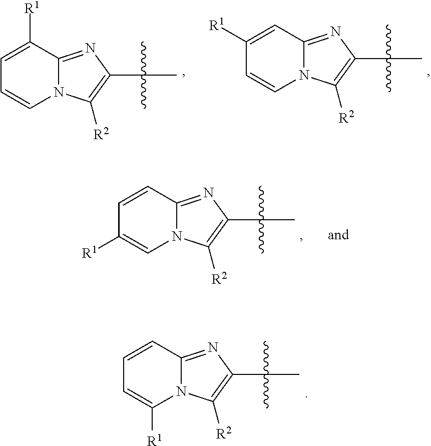

In some embodiments,

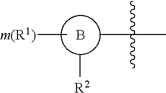

is selected from the

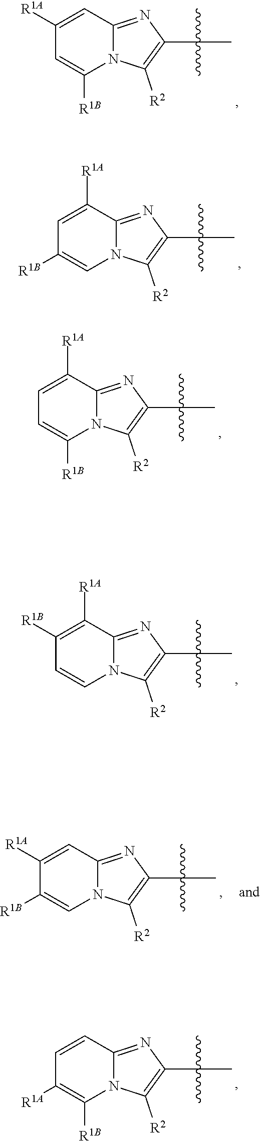

wherein R^{1A} and R^{1B} are independently selected from R¹.

In some embodiments,

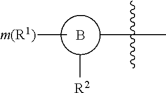

is

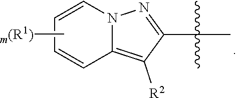

In some embodiments

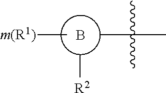

is selected from the group consisting of

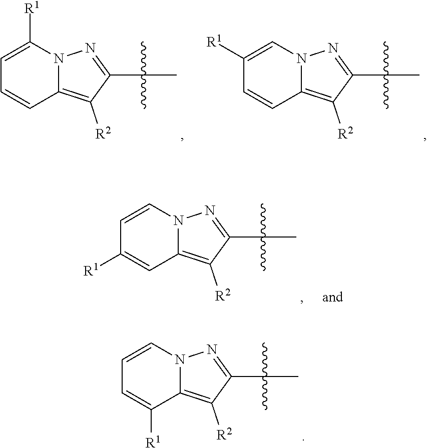

In some embodiments,

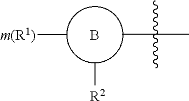

is selected from the group consisting of

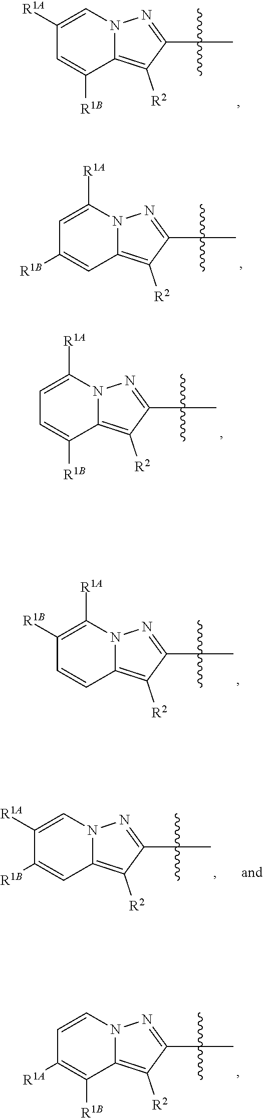

wherein R^{1A} and R^{IB} are independently selected from R¹.

In some embodiments, each R¹ is an independently selected halogen. In some embodiments, each R¹ is independently selected from fluoro and chloro. In some embodiments, each R¹ is independently selected from fluoro and bromo. In some embodiments, each R¹ is fluoro. In some embodiments, at least one R¹ is an independently selected halogen. In some embodiments, at least one R¹ is independently selected from fluoro and chloro. In some embodiments, at least one R¹ is fluoro.

In some embodiments, at least one R¹ is cyano. In some embodiments, at least one R¹ is hydroxyl. In some embodiments, at least one R¹ is C1-C6 alkyl optionally substituted with hydroxyl. In some embodiments, at least one R¹ is C1-C6 alkyl substituted with hydroxyl. In some embodiments, at least one R¹ is C1-C3 alkyl substituted with hydroxyl. In some embodiments, at least one R¹ is hydroxymethyl. In some embodiments, at least one R¹ is unsubstituted C1-C6 alkyl. In some embodiments, at least one R¹ is methyl. In some embodiments, at least one R¹ is C3-C6 cycloalkyl. In some embodiments, at least one R¹ is cyclopropyl.

In some embodiments, m is 2; one R¹ is halogen; and the other R¹ is C1-C6 alkyl. In some embodiments, m is 2; one R¹ is fluoro; and the other R¹ is methyl In some embodiments, m is 2; one R¹ is halogen; and the other R¹ is C3-C6 cycloalkyl. In some embodiments, m is 2; one R¹ is halogen; and the other R¹ is cyclopropyl. In some embodiments, m is 2; one R¹ is fluoro; and the other R¹ is cyano. In some embodiments, m is 2; one R¹ is halogen; and the other R¹ is halogen. In some embodiments, m is 2; one R¹ is fluoro; and the other R¹ is fluoro.

In some embodiments, R² is hydroxyl. In some embodiments, R² is C1-C6 alkyl optionally substituted with hydroxyl. In some embodiments, R² is C1-C6 alkyl substituted with hydroxyl. In some embodiments, R² is C1-C3 alkyl substituted with hydroxyl. In some embodiments, R² is hydroxymethyl. In some embodiments, R² is an unsubstituted C1-C6 alkyl. In some embodiments, R² is unsubstituted C1-C3 alkyl. In some embodiments, R² is methyl.

In some embodiments, R² is a C1-C6 haloalkyl. In some embodiments, R² is a C1-C3 haloalkyl. In some embodiments, R² is difluoromethyl. In some embodiments, R² is trifluoromethyl.

In some embodiments, R² is halogen. In some embodiments, R² is fluoro. In some embodiments, R² is chloro.

In some embodiments, R² is C3-C6 cycloalkyl optionally substituted with 1 or 2 fluoro. In some embodiments, R² is C3-C6 cycloalkyl substituted with 1 or 2 fluoro. In some embodiments, R² is C3-C6 cycloalkyl substituted with 1 fluoro. In some embodiments, R² is C3-C6 cycloalkyl substituted with 2 fluoro. In some embodiments, R² is C3-C4 cycloalkyl substituted with 1 fluoro. In some embodiments, R² is C3-C4 cycloalkyl substituted with 2 fluoro. In some embodiments, R² is an unsubstituted C3-C6 cycloalkyl.

In some embodiments, R³ is a C1-C6 alkyl. In some embodiments, R³ is a C1-C3 alkyl. In some embodiments, R³ is methyl, ethyl, t-butyl, or isopropyl. In some embodiments, R³ is methyl, ethyl, or isopropyl. In some embodiments, R³ is methyl. In some embodiments, R³ is ethyl. In some embodiments, R³ is isopropyl.

In some embodiments, R³ is a C1-C6 haloalkyl. In some embodiments, R³ is a C1-C3 haloalkyl. In some embodiments, R³ is difluoromethyl. In some embodiments, R³ is trifluoromethyl.

In some embodiments, R³ is C3-C6 cycloalkyl optionally substituted with 1 or 2 substituents independently selected from fluoro and C1-C6 alkyl. In some embodiments, R³ is C3-C6 cycloalkyl optionally substituted with 1 or 2 fluoro. In some embodiments, R³ is C3-C6 cycloalkyl substituted with 1 or 2 fluoro. In some embodiments, R³ is C3-C6 cycloalkyl substituted with 1 fluoro. In some embodiments, R³ is C3-C6 cycloalkyl substituted with 1 fluoro at the position of the C3-C6 cycloalkyl that is bonded to the methine of Formula (I). In some embodiments, R³ is 2,2-difluorocyclopropyl or 3,3-difluorocyclopropyl. In some embodiments, R³ is C3-C6 cycloalkyl optionally substituted with 1 or 2 methyl. In some embodiments, R³ is C3-C6 cycloalkyl substituted with 1 or 2 methyl. In some embodiments, R³ is C3-C6 cycloalkyl substituted with 1 methyl. In some embodiments, R³ is C3-C6 cycloalkyl substituted with 1 methyl at the position of the C3-C6 cycloalkyl that is bonded to the methine of Formula (I). In some embodiments, R³ is an unsubstituted C3-C6 cycloalkyl. In some embodiments, the R³ C3-C6 cycloalkyl is cyclopropyl. In some embodiments, R³ is cyclopropyl. In some embodiments, R³ is cyclobutyl. In some embodiments, R³ is cyclopentyl. In some embodiments, R³ is cyclohexyl.

In some embodiments, Ring A is a 6-10 membered aryl. In some embodiments, Ring A is phenyl, naphthyl, or tetrahydronaphthyl. In some embodiments, Ring A is phenyl.

In some embodiments, Ring A is a C3-C8 cycloalkyl. In some embodiments, Ring A is a C5-C6 cycloalkyl. In some embodiments, Ring A is cyclohexyl.

In some embodiments, Ring A is a 5-10 membered heteroaryl. In some embodiments, Ring A is a 9-10 membered heteroaryl. In some embodiments, Ring A is a 9 membered heteroaryl. In some embodiments, Ring A is a 9 membered heteroaryl, wherein the point of attachment to the urea nitrogen atom in Formula (I) is on a 6-membered ring of Ring A. In some embodiments, Ring A is a 9 membered heteroaryl, wherein the point of attachment to the urea nitrogen atom in Formula (I) is on a 5-membered ring of Ring A.

In some embodiments, Ring A is benzimidazolyl, indazolyl, indolyl, quinazolone, isobenzofuranonyl, isoindolinonyl, imidazo[1,2-a]pyridinyl, or imidazo[1,2-a]pyrimidinyl. In some embodiments, Ring A is benzimidazolyl, indazolyl, indolyl, quinazolone, isobenzofuranonyl, isoindolinonyl, 5,6,7,8-tetrahydroimidazo[1,5-a]pyridin-6-yl, or imidazo[1,2-a]pyridinyl. In some embodiments, Ring A is benzimidazolyl, indazolyl, indolyl, or imidazo[1,2-a]pyridinyl. In some embodiments, Ring A is 2-benzimidazolyl, 5-indazolyl, 2-indolyl, 7-imidazo[1,2-a]pyridinyl,

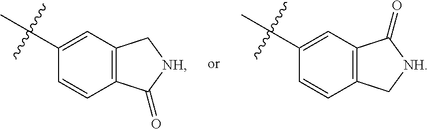 [SMILES: *c1ccc2c(c1)CNC2=O ; "or" ; *c1ccc2c(c1)C(=O)NC2]

In some embodiments, Ring A is selected from the group consisting of

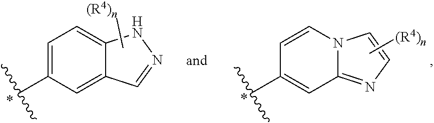

wherein “*” indicates the attachment point to the urea nitrogen atom in Formula (I).

In some embodiments, Ring A is a 5-6 membered heteroaryl. In some embodiments, Ring A is selected from the group consisting of pyrrolyl, pyrazolyl, imidazolyl, triazolyl, tetrazolyl, furanyl, thiophenyl, oxazolyl, isoxazolyl, isothiazolyl, thiazolyl, furzanyl, oxadiazolyl, thiadiazolyl, oxatriazolyl, and thiatriazolyl. In some embodiments, Ring A is selected from the groups consisting of pyridinyl, pyrimidinyl, pyrazinyl, pyridazinyl, and triazinyl. In some embodiments, Ring A is pyrimidinyl, pyridyl, thiazolyl, thiophenyl, or pyrazolyl. In some embodiments, Ring A is pyrimidinyl, pyridyl, or pyrazolyl. In some embodiments, Ring A is 5-pyrimidinyl, 3-pyridyl, or 4-pyrazolyl. In some embodiments, Ring A is selected from the group consisting of

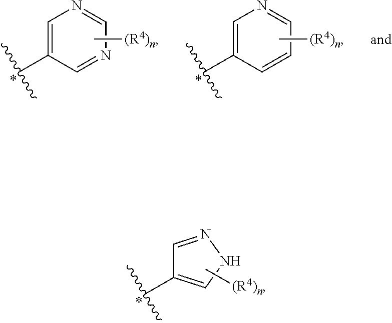

wherein “*” indicates the attachment point to the urea nitrogen atom in Formula (I). In some embodiments, Ring A is pyrimidinyl. In some embodiments, Ring A is 5-pyrimidinyl. In some embodiments, Ring A is

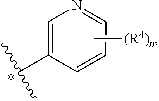

wherein “*” indicates the attachment point to the urea nitrogen atom in Formula (I). In some embodiments, Ring A is a 4-10 membered heterocyclyl. In some embodiments, Ring A is a 6-9 membered heterocyclyl. In some embodiments, Ring A is piperidinyl, isoindolinone, or tetrahydro-2H-thiopyranyl-1,1-dioxide.

In some embodiments, Ring A is 2-benzimidazolyl, 5-indazolyl, 2-indolyl, 7-imidazo[1,2-a]pyridinyl,

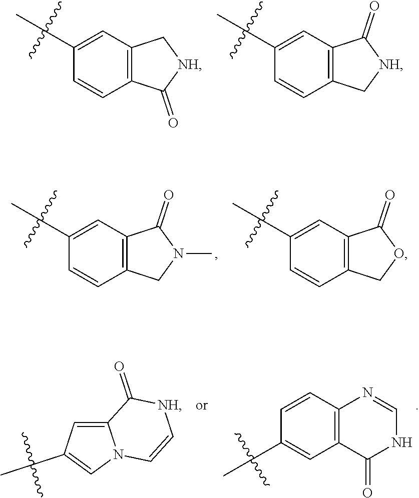 [SMILES: *c1ccc2c(c1)CNC2=O ; *c1ccc2c(c1)C(=O)NC2 ; *c1ccc2c(c1)C(=O)N(C)C2 ; *c1ccc2c(c1)C(=O)OC2 ; *c1cc2c(=O)[nH]ccn2c1 ; *c1ccc2nc[nH]c(=O)c2c1]

In some embodiments, Ring A is 2-benzimidazolyl, 5-indazolyl, 2-indolyl, 7-imidazo[1,2-a]pyridinyl,

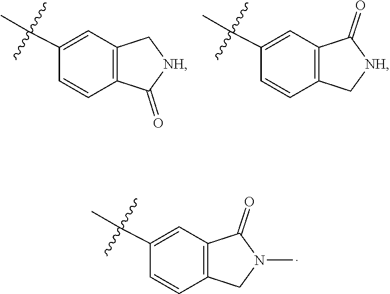 [SMILES: *c1ccc2c(c1)CNC2=O ; *c1ccc2c(c1)C(=O)NC2 ; *c1ccc2c(c1)C(=O)N(C)C2]

In some embodiments, Ring A is selected from the group consisting of 3-piperidinyl,

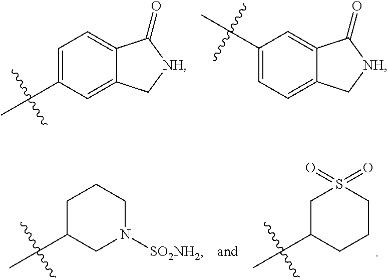 [SMILES: *c1ccc2c(c1)CNC2=O ; *c1ccc2c(c1)C(=O)NC2 ; *C1CCCN(S(N)(=O)=O)C1 ; *C1CCCS(=O)(=O)C1]

In some embodiments, n is 0. In some embodiments, n is 1. In some embodiments, n is 2.

In some embodiments, one R⁴ is C1-C6 alkyl optionally substituted with 1 or 2 hydroxyl or —NR^{A}R^{B}. In some embodiments, one R⁴ is C1-C4 alkyl optionally substituted with 1 or 2 hydroxyl or —NR^{A}R^{B}. In some embodiments, one R⁴ is C1-C6 alkyl substituted with 1 or 2 hydroxyl. In some embodiments, one R⁴ is C1-C6 alkyl substituted with 1 hydroxyl. In some embodiments, one R⁴ is C1-C6 alkyl substituted with 2 hydroxyl. In some embodiments, one R⁴ is C1-C3 alkyl substituted with 2 hydroxyl. In some embodiments, one R⁴ is C1-C6 alkyl substituted with —NR^{A}R^{B}. In some embodiments, one R⁴ is C1-C3 alkyl substituted with —NR^{A}R^{B}. In some embodiments, one R⁴ is methyl or ethyl substituted with —NR^{A}R^{B}. In some embodiments, one R⁴ is C1-C6 alkyl substituted with hydroxyl and —NR^{A}R^{B}. In some embodiments, one R⁴ is unsubstituted C1-C6 alkyl. In some embodiments, one R⁴ is C1-C4 alkyl. In some embodiments, one R⁴ is t-butyl. In some embodiments, one R⁴ is methyl, ethyl, or iso-propyl.

In some embodiments, one R⁴ is C1-C6 alkoxy optionally substituted with 1-2 substituents independently selected from hydroxyl and C3-C6 cycloalkyl. In some embodiments, one R⁴ is C1-C6 alkoxy substituted with 1-2 substituents independently selected from hydroxyl and C3-C6 cycloalkyl. In some embodiments, one R⁴ is C1-C6 alkoxy substituted with 1-2 substituents independently selected from hydroxyl and cyclopropyl. In some embodiments, one R⁴ is C1-C6 alkoxy substituted with hydroxyl. In some embodiments, one R⁴ is C1-C6 alkoxy substituted with C3-C6 cycloalkyl. In some embodiments, one R⁴ is C1-C6 alkoxy substituted with cyclopropyl. In some embodiments, R⁴ is C1-C6 alkoxy. In some embodiments, R⁴ is C1-C3 alkoxy. In some embodiments, one R⁴ is methoxy.

In some embodiments, one R⁴ is C1-C6 haloalkyl. In some embodiments, one R⁴ is C1-C3 haloalkyl. In some embodiments, one R⁴ is difluoromethyl. In some embodiments, one R⁴ is trifluoromethyl.

In some embodiments, one R⁴ is hydroxyl. In some embodiments, one R⁴ is cyano. In some embodiments, one R⁴ is —CO₂H. In some embodiments, one R⁴ is halogen. In some embodiments, one R⁴ is fluoro. In some embodiments, one R⁴ is chloro.

In some embodiments, one R⁴ is —NR^{A}R^{B}. In some embodiments, one R⁴ is ═NR^{A2}.

In some embodiments, R^{A} and R^{B} are each hydrogen. In some embodiments, one of R^{A} and R^{B} is hydrogen and the other of R^{A} and R^{B} is C1-C6 alkyl optionally substituted with hydroxyl or —C(═O)NR^{B2}R^{C2}. In some embodiments, one of R^{A} and R^{B} is hydrogen and the other of R^{A} and R^{B} is C1-C6 alkyl optionally substituted with hydroxyl or —C(═O)NH₂. In some embodiments, one of R^{A} and R^{B} is hydrogen and the other of R^{A} and R^{B} is —C(═O)O(C1-C6 alkyl). In some embodiments, one of R^{A} and R^{B} is hydrogen and the other of R^{A} and R^{B} is —C(═O)OCH₃. In some embodiments, one of R^{A} and R^{B} is hydrogen and the other of R^{A} and R^{B} is 4-6 membered heterocyclyl (e.g., oxetanyl), In some embodiments, one of R^{A} and R^{B} is hydrogen and the other of R^{A} and R^{B} is C1-C6 alkyl optionally substituted with hydroxyl. In some embodiments, one of R^{A} and R^{B} is hydrogen and the other of R^{A} and R^{B} is C1-C6 alkyl substituted with hydroxyl. In some embodiments, one of R^{A} and R^{B} is hydrogen and the other of R^{A} and R^{B} is C1-C6 alkyl. In some embodiments, one of R^{A} and R^{B} is hydrogen and the other of R^{A} and R^{B} is C1-C3 alkyl optionally substituted with hydroxyl. In some embodiments, one of R^{A} and R^{B} is hydrogen and the other of R^{A} and R^{B} is C1-C3 alkyl substituted with hydroxyl. In some embodiments, one of R^{A} and R^{B} is hydrogen and the other of R^{A} and R^{B} is ethyl substituted with hydroxyl (e.g., 2-hydroxy-1-propyl). In some embodiments, one of R^{A} and R^{B} is hydrogen and the other of R^{A} and R^{B} is propyl substituted with hydroxyl (e.g., 3-hydroxy-1-propyl, 2-hydroxy-1-propyl or 1-hydroxy-2-propyl). In some embodiments, one of R^{A} and R^{B} is hydrogen and the other of R^{A} and R^{B} is butyl substituted with hydroxyl (e.g., 2-hydroxy-2-methyl-1-propyl). In some embodiments, one of R^{A} and R^{B} is hydrogen and the other of R^{A} and R^{B} is C1-C3 alkyl. In some embodiments, one of R^{A} and R^{B} is hydrogen and the other of R^{A} and R^{B} is methyl. In some embodiments, R^{A} and R^{B} are each C1-C6 alkyl optionally substituted with hydroxyl. In some embodiments, R^{A} and R^{B} are each C1-C6 alkyl substituted with hydroxyl. In some embodiments, one of R^{A} and R^{B} is C1-C3 alkyl and the other of R^{A} and R^{B} is C1-C3 alkyl substituted with hydroxyl. In some embodiments, one of R^{A} and R^{B} is methyl and the other of R^{A} and R^{B} is C1-C3 alkyl substituted with hydroxyl. In some embodiments, one of R^{A} and R^{B} is methyl and the other of R^{A} and R^{B} is ethyl substituted with hydroxyl (e.g., 2-hydroxy-1-propyl). In some embodiments, R^{A} and R^{B} are each C1-C6 alkyl. In some embodiments, R^{A} and R^{B} are each C1-C3 alkyl. In some embodiments, R^{A} and R^{B} are each methyl.

In some embodiments, both of R^{B2} and R^{C2} are hydrogen. In some embodiments, one of R^{B2} and R^{C2} is hydrogen and the other of R^{B2} and R^{C2} is C1-C6 alkyl. In some embodiments, one of R^{B2} and R^{C2} is hydrogen and the other of R^{B2} and R^{C2} is methyl. In some embodiments, both of R^{B2} and R^{C2} are methyl.

In some embodiments, one of R^{A} and R^{B} is hydrogen and the other of R^{A} and R^{B} is C1-C6 haloalkyl. In some embodiments, one of R^{A} and R^{B} is hydrogen and the other of R^{A} and R^{B} is C1-C3 haloalkyl. In some embodiments, R^{A} and R^{B} are each C1-C6 haloalkyl. In some embodiments, R^{A} and R^{B} are each C1-C3 haloalkyl.

In some embodiments, one of R^{A} and R^{B} is C1-C6 alkyl and the other of one of R^{A} and R^{B}is C1-C6 haloalkyl.

In some embodiments, one R⁴ is —C(═O)NR^{C}R^{D}.

In some embodiments, R^{C} and R^{D} are each hydrogen. In some embodiments, one of R^{C} and R^{D} is hydrogen and the other of R^{C} and R^{D} is C1-C6 alkyl. In some embodiments, one of R^{C} and R^{D} is hydrogen and the other of R^{C} and R^{D} is C1-C3 alkyl. In some embodiments, one of R^{C} and R^{D} is hydrogen and the other of R^{C} and R^{D} is methyl. In some embodiments, R^{C} and R^{D} are each C1-C6 alkyl. In some embodiments, R^{C} and R^{D} are each C1-C3 alkyl. In some embodiments, R^{C} and R^{D} are each methyl. In some embodiments, one of R^{C} and R^{D} is C1-C6 alkyl and the other of R^{C} and R^{D} is C1-C3 alkyl.

In some embodiments, one of R^{C} and R^{D} is hydrogen and the other of R^{C} and R^{D} is C1-C6 haloalkyl. In some embodiments, one of R^{C} and R^{D} is hydrogen and the other of R^{C} and R^{D} is C1-C3 haloalkyl. In some embodiments, R^{C} and R^{D} are each is C1-C6 haloalkyl. In some embodiments, one of R^{C} and R^{D} is C1-C6 alkyl and the other of R^{C} and R^{D} is C1-C6 haloalkyl.

In some embodiments, R^{C} and R^{D}, together with the nitrogen atom to which they are attached form a 4-10 membered heterocyclyl optionally substituted with 1-2 substituents independently selected from hydroxyl, halogen, —C(═O)NR^{B1}R^{C1}, —SO₂(C1-C6 alkyl), —CO₂H, C1-C6 alkyl optionally substituted with hydroxyl, C1-C6 alkoxy, and C1-C6 haloalkoxy. In some embodiments, R^{C} and R^{D}, together with the nitrogen atom to which they are attached form a 4-10 membered heterocyclyl substituted with 1-2 substituents independently selected from hydroxyl, halogen, —C(═O)NR^{B1}R^{C1}, —SO₂(C1-C6 alkyl), —CO₂H, C1-C6 alkyl optionally substituted with hydroxyl, C1-C6 alkoxy, and C1-C6 haloalkoxy.

In some embodiments, R^{B1} and R^{C1} are each hydrogen. In some embodiments, one of R^{B1} and R^{C1} is hydrogen and the other of R^{B1} and R^{C1} is C1-C6 alkyl. In some embodiments, one of R^{B1} and R^{C1} is hydrogen and the other of R^{B1} and R^{C1} is methyl. In some embodiments, R^{B1} and R^{C1} are each independently selected C1-C6 alkyl. In some embodiments, R^{B1} and R^{C1} are each methyl.

In some embodiments, R^{C} and R^{D}, together with the nitrogen atom to which they are attached form a 4-6 membered heterocyclyl. In some embodiments, R^{C} and R^{D}, together with the nitrogen atom to which they are attached form azetidine or piperazine.

In some embodiments, one R⁴ is —SO₂(NR^{E}R^{F}). In some embodiments, R^{E} and R^{F} are each hydrogen. In some embodiments, one of R^{E} and R^{F} is hydrogen and the other of R^{E} and R^{F} is C1-C6 alkyl. In some embodiments, one of R^{E} and R^{F} is hydrogen and the other of R^{E} and R^{F} is C1-C3 alkyl. In some embodiments, one of R^{E} and R^{F} is hydrogen and the other of R^{E} and R^{F} is methyl. In some embodiments, R^{E} and R^{F} are each is C1-C6 alkyl. In some embodiments, R^{E} and R^{F} are each is C1-C3 alkyl. In some embodiments, R^{E} and R^{F} are each methyl.

In some embodiments, one of R^{E} and R^{F} is hydrogen and the other of R^{E} and R^{F} is C1-C6 haloalkyl. In some embodiments, one of R^{E} and R^{F} is hydrogen and the other of R^{E} and R^{F} is C1-C3 haloalkyl. In some embodiments, R^{E} and R^{F} are each C1-C6 haloalkyl. In some embodiments, one of R^{E} and R^{F} is C1-C6 alkyl and the other of R^{E} and R^{F} is C1-C6 haloalkyl.

In some embodiments, one R⁴ is —SO₂(C1-C6 alkyl). In some embodiments, one R⁴ is —SO₂(C1-C3 alkyl). In some embodiments, one R⁴ is —SO₂Et. In some embodiments, one R⁴ is —SO₂Me.

In some embodiments, one R⁴ is —S(═O)(═NH)(C1-C6 alkyl). In some embodiments, one R⁴ is —S(═O)(═NH)(C1-C3 alkyl). In some embodiments, one R⁴ is —S(═O)(═NH)Me.

In some embodiments, one R⁴ is —C(═O)(C1-C6 alkyl). In some embodiments, one R⁴ is —C(═O)(C1-C3 alkyl). In some embodiments, one R⁴ is —C(═O)Me.

In some embodiments, one R⁴ is —CO₂(C1-C6 alkyl). In some embodiments, one R⁴ is —CO₂(C1-C3 alkyl). In some embodiments, one R⁴ is —CO2Me.

In some embodiments, one R⁴ is 5-6 membered heteroaryl optionally substituted with C1-C6 alkyl. In some embodiments, one R⁴ is 5-6 membered heteroaryl substituted with C1-C6 alkyl. In some embodiments, one R⁴ is 5-6 membered heteroaryl. In some embodiments, one R⁴ is selected from the group consisting of pyrrolyl, pyrazolyl, imidazolyl, triazolyl, tetrazolyl, furanyl, thiophenyl, oxazolyl, isoxazolyl, isothiazolyl, thiazolyl, furanyl, oxadiazolyl, thiadiazolyl, oxatriazolyl, and thiatriazolyl, pyridinyl, pyrimidinyl, pyrazinyl, pyridazinyl, and triazinyl. In some embodiments, one R⁴ is tetrazolyl substituted with methyl. In some embodiments, one R⁴ is pyrazolyl. In some embodiments, one R⁴ is unsubstituted pyrazolyl. In some embodiments, one R⁴ is 1-pyrazolyl.

In some embodiments, one R⁴ is 3-9 membered heterocyclyl optionally substituted with 1 or 2 independently selected R^{G}. In some embodiments, one R⁴ is 3 membered heterocyclyl optionally substituted with 1 or 2 independently selected R^{G}. In some embodiments, one R⁴ is 4 membered heterocyclyl optionally substituted with 1 or 2 independently selected R^{G}. In some embodiments, one R⁴ is 5 membered heterocyclyl optionally substituted with 1 or 2 independently selected R^{G}. In some embodiments, one R⁴ is 7-9 membered heterocyclyl optionally substituted with 1 or 2 independently selected R^{G}. In some embodiments, the R⁴ heterocyclyl is a spirocycle. In some embodiments, one R⁴ is 3-6 membered heterocyclyl optionally substituted with 1 or 2 independently selected R^{G}. In some embodiments, one R⁴ is 3-6 membered heterocyclyl substituted with 1 or 2 independently selected R^{G}. In some embodiments, one R⁴ is 3-6 membered heterocyclyl substituted with 1 R^{G}. In some embodiments, one R⁴ is 3-6 membered heterocyclyl substituted with 2 independently selected R^{G}. In some embodiments, one R⁴ is an unsubstituted 3-6 membered heterocyclyl.

In some embodiments, one R⁴ is a C3-C6 cycloalkyl optionally substituted with 1 or 2 independently selected R^{G}. In some embodiments, one R⁴ is C3-C6 cycloalkyl substituted with 1 or 2 independently selected R^{G}. In some embodiments, one R⁴ is C3-C6 cycloalkyl substituted with 1 R^{G}. In some embodiments, one R⁴ is C3-C6 cycloalkyl substituted with 2 independently selected R^{G}. In some embodiments, one R⁴ is an unsubstituted C3-C6 cycloalkyl.

In some embodiments, the 1 or 2 independently selected R^{G} is 1 R^{G}. In some embodiments, the 1 or 2 independently selected R^{G} are 2 independently selected R^{G}. In some embodiments, when 2 R^{G} are present, they are bonded to the same atom, valency permitting. In some embodiments, when 2 R^{G} are present, they are bonded to adjacent atoms, valency permitting. In some embodiments, when 2 R^{G} are present, the 2 R^{G} are different. In some embodiments, when 2 R^{G} are present, the 2 R^{G} are the same. In some embodiments, one R^{G} is fluoro. In some embodiments, one R^{G} is cyano. In some embodiments, one R^{G} is hydroxyl. In some embodiments, one R^{G} is C1-C6 alkyl optionally substituted with hydroxyl. In some embodiments, one R^{G} is 2-hydroxy-2-propyl. In some embodiments, one R^{G} is C1-C6 alkyl. In some embodiments, one R^{G} is C1-C3 alkyl. In some embodiments, one R^{G} is methyl. In some embodiments, one R^{G} is ethyl.

In some embodiments, one R^{G} is C1-C6 alkoxy. In some embodiments, one R^{G} is C1-C3 alkoxy. In some embodiments, one R^{G} is methoxy.

In some embodiments, one R^{G} is —NR^{A1}R^{B1}. In some embodiments, R^{A1} and R^{B1} are each hydrogen. In some embodiments, one of R^{A1} and R^{B1} is hydrogen and the other of R^{A1} and R^{B1} is C1-C6 alkyl. In some embodiments, one of R^{A1} and R^{B1} is hydrogen and the other of R^{A1} and R^{B1}is C1-C3 alkyl. In some embodiments, one of R^{A1} and R^{B1} is hydrogen and the other of R^{A1} and R^{B1} is methyl. In some embodiments, R^{A1} and R^{B1} are each C1-C6 alkyl. In some embodiments, R^{A1} and R^{B1} are each methyl.

In some embodiments, one of R^{A1} and R^{B1} is hydrogen and the other of R^{A1} and R^{B1} is C1-C6 haloalkyl. In some embodiments, one of R^{A1} and R^{B1} is hydrogen and the other of R^{A1} and R^{B1}is C1-C3 haloalkyl. In some embodiments, R^{A1} and R^{B1} are each C1-C6 haloalkyl. In some embodiments, one of R^{A1} and R^{B1} is C1-C6 alkyl and the other of R^{A1} and R^{B1} is C1-C6 haloalkyl.

In some embodiments, one R^{G} is ═NR^{A2}. In some embodiments, one R^{G} is ═NH. In some embodiments, R^{A2} is hydrogen. In some embodiments, R^{A2} is C1-C6 alkyl. In some embodiments, R^{A2} is methyl.

In some embodiments, one R^{G} is —C(═O)NR^{C1}R^{D1}. In some embodiments, one R^{G} is —CO₂NH₂. In some embodiments, one R^{G} is —CO₂NHCH₃. In some embodiments, R^{C1} and R^{D1} are each is hydrogen. In some embodiments, one of R^{C1} and R^{D1} is hydrogen and the other of R^{C1} and R^{D1} is C1-C6 alkyl. In some embodiments, one of R^{C1} and R^{D1} is hydrogen and the other of R^{C1} and R^{D1} is C1-C3 alkyl. In some embodiments, one of R^{C1} and R^{D1} is hydrogen and the other of R^{C1} and R^{D1} is methyl. In some embodiments, R^{C1} and R^{D1} are each is C1-C6 alkyl. In some embodiments, R^{C1} and R^{D1} are each is C1-C3 alkyl. In some embodiments, R^{C1} and R^{D1} are each is methyl.

In some embodiments, one of R^{C1} and R^{D1} is hydrogen and the other of R^{C1} and R^{D1} is C1-C6 haloalkyl. In some embodiments, one of R^{C1} and R^{D1} is hydrogen and the other of R^{C1} and R^{D1}is C1-C3 haloalkyl. In some embodiments, R^{C1} and R^{D1} are each is C1-C6 haloalkyl. In some embodiments, one of R^{C1} and R^{D1} is C1-C6 alkyl and the other of R^{C1} and R^{D1} is C1-C6 haloalkyl.

In some embodiments, one R^{G} is —CO₂(C1-C6 alkyl). In some embodiments, one R^{G} is —CO₂CH₃. In some embodiments, one R^{G} is C1-C6 haloalkyl. In some embodiments, one R^{G} is trifluoromethyl. In some embodiments, one R^{G} is difluoromethyl. In some embodiments, one R^{G}is C3-C6 cycloalkyl. In some embodiments, one R^{G} is cyclopropyl. In some embodiments, one R^{G} is —CO₂H.

In some embodiments, one R^{G} is C1-C6 haloalkoxy. In some embodiments, one R^{G} is C1-C3 haloalkoxy. In some embodiments, one R^{G} is difluoromethoxy. In some embodiments, one R^{G} is trifluoromethoxy.

In some embodiments, one R^{G} is —SO₂(C1-C6 alkyl). In some embodiments, one R^{G} is —SO₂CH₃.

In some embodiments, the R⁴ 3-9 membered heterocyclyl is a 3-6 membered heterocyclyl. In some embodiments, the R⁴ 3-6 membered heterocyclyl is a 5-6 membered heterocyclyl. In some embodiments, the R⁴ 3-6 membered heterocyclyl is azetidinyl, azetidin-2-onyl, morpholinyl, piperazinyl, or tetrahydropyranyl. In some embodiments, the R⁴ 3-6 membered heterocyclyl is 1-azetidinyl, 1-azetidin-2-onyl, 1-piperazinyl, 1-morpholinyl, or 4-tetrahydropyranyl. In some embodiments, the R⁴ 3-9 membered heterocyclyl is selected from the group consisting of

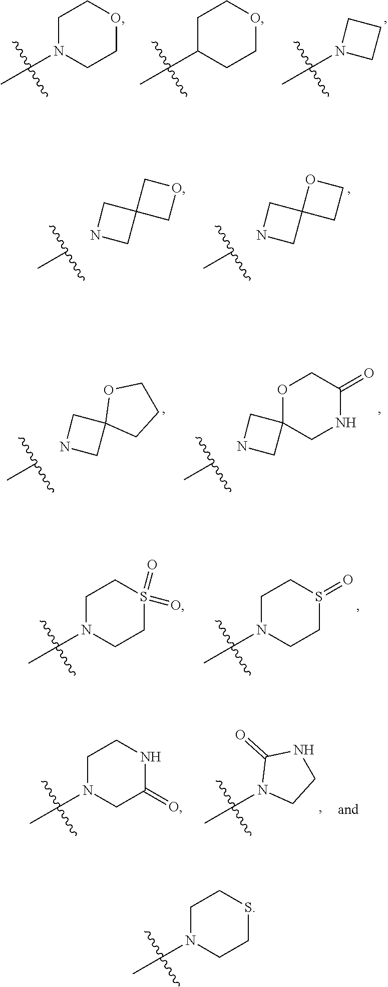

In some embodiments, the R⁴ 3-9 membered heterocyclyl (e.g., the R⁴ 3-6 membered heterocyclyl) is

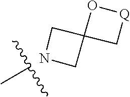

wherein Q is a C1-C3 alkylene in which one or more carbons is optionally replaced by —C(═O)—, NH, 0, or S. In some embodiments, Q is a C1-C3 alkylene in which one or more carbons is optionally replaced by —C(═O)— or NH. In some embodiments, Q is a C1-C2 alkylene in which one or more carbons is optionally replaced by —C(═O)— or NH. In some embodiments, the R⁴ 3-9 membered heterocyclyl is selected from the group consisting of N

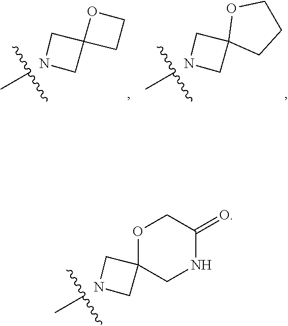

In some embodiments, one R⁴ is unsubstituted 3-6 membered heterocyclyl. In some embodiments, R⁴ 3-6 membered heterocyclyl is a 5-6 membered heterocyclyl. In some embodiments, R⁴ is azetidinyl, morpholinyl, or tetrahydropyranyl. In some embodiments, R⁴ is selected from the group consisting of

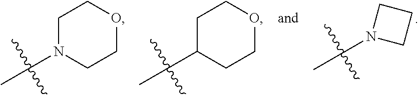 [SMILES: *N1CCOCC1 ; *C1CCOCC1 ; *N1CCC1]

In some embodiments,

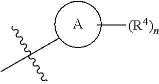

is

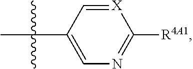

wherein: X is selected from N and CR^{4A2}; R^{4A1} and R^{4A2} are independently selected from hydrogen, C1-C3 alkyl optionally substituted with —NR^{A}R^{B}, methoxy, C1-C3 haloalkyl, hydroxyl, cyano, —CO₂H, —NR^{A}R^{B}, —C(═O)NR^{C}R^{D}, —SO₂(NR^{E}R^{F}), —SO₂(C1-C6 alkyl), and 3-6 membered heterocyclyl optionally substituted with 1 or 2 independently selected R^{G}, and C3-C6 cycloalkyl optionally substituted with 1 or 2 independently selected R^{G}. In some embodiments, X is N. In some embodiments, X is CR^{4A2}. In some embodiments, R^{4A1} and, when present, R^{4A2} are independently selected from hydrogen, methyl, ethyl, isopropyl, difluoromethyl, trifluoromethyl, cyano, hydroxyl, methoxy, amino, —C(═O)NH₂, —C(═O)NHMe, —SO₂NH₂, —SO₂Me, and azetidinyl optionally substituted with 1-2 independently selected fluoro, hydroxyl, or methyl. In some embodiments, R^{C} and R^{D}, together with the nitrogen atom to which they are attached form a 4-6 membered heterocyclyl. In some embodiments, X is N and R^{4A1} is 3-6 membered heterocyclyl optionally substituted with 1 or 2 independently selected R^{G}. In some embodiments, R^{C} and R^{D}, together with the nitrogen atom to which they are attached form azetidine or piperazine.

In some embodiments, X is N; and R^{4A1} is selected from amino or an azetidinyl optionally substituted with 1-2 independently selected fluoro, hydroxyl, or methyl.

In some embodiments,

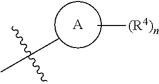

is

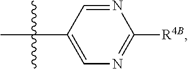

wherein: R^{4B} is selected from —NR^{A}R^{B} and 4-6 membered heterocyclyl comprising one nitrogen ring member and optionally substituted with 1-2 independently selected R^{G1}; wherein R^{G1} is selected from fluoro, hydroxyl, C1-C6 haloalkyl, and C1-C6 alkyl. In some embodiments, R^{G1} is selected from fluoro, hydroxyl, and C1-C6 alkyl.

In some embodiments,

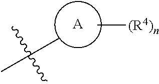

is

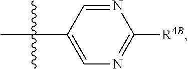

wherein: R^{4B} is selected from —NR^{A}R^{B} and 4-6 membered heterocyclyl comprising one nitrogen ring member and optionally substituted with 1-2 independently selected R^{G1}; wherein R^{G1} is selected from fluoro, hydroxyl, methoxy, methyl, ethyl, amino, hydroxymethyl, 2-hydroxy-2-propyl, —C(O)Me, —C(O)NH₂, ═NH, difluoromethoxy, —S(O)₂Me, —CO₂H, C1-C6 haloalkyl, and C1-C6 alkyl. In some embodiments, R^{G1} is selected from fluoro, hydroxyl, methoxy, methyl, ethyl, hydroxymethyl, 2-hydroxy-2-propyl, —C(O)Me, —C(O)NH₂, ═NH, difluoromethoxy, —S(O)₂Me, —CO₂H, C1-C6 haloalkyl, and C1-C6 alkyl. In some embodiments, R^{G1} is selected from fluoro, hydroxyl, and C1-C6 alkyl.

In some embodiments, R^{A} and R^{B} are each hydrogen. In some embodiments, one of R^{A} and R^{B} is hydrogen and the other of R^{A} and R^{B} is C1-C6 alkyl optionally substituted with hydroxyl. In some embodiments, one of R^{A} and R^{B} is hydrogen and the other of R^{A} and R^{B} is C1-C6 alkyl substituted with hydroxyl. In some embodiments, one of R^{A} and R^{B} is hydrogen and the other of R^{A} and R^{B} is C1-C6 alkyl. In some embodiments, one of R^{A} and R^{B} is hydrogen and the other of R^{A} and R^{B} is C1-C3 alkyl optionally substituted with hydroxyl. In some embodiments, one of R^{A} and R^{B} is hydrogen and the other of R^{A} and R^{B} is C1-C3 alkyl substituted with hydroxyl. In some embodiments, one of R^{A} and R^{B} is hydrogen and the other of R^{A} and R^{B} is ethyl substituted with hydroxyl (e.g., 2-hydroxy-1-propyl. In some embodiments, one of R^{A} and R^{B} is hydrogen and the other of R^{A} and R^{B} is propyl substituted with hydroxyl (e.g., 2-hydroxyl-propyl or 1-hydroxy-2-propyl). In some embodiments, one of R^{A} and R^{B} is hydrogen and the other of R^{A} and R^{B} is C1-C3 alkyl. In some embodiments, one of R^{A} and R^{B} is hydrogen and the other of R^{A} and R^{B} is methyl. In some embodiments, R^{A} and R^{B} are each C1-C6 alkyl optionally substituted with hydroxyl. In some embodiments, R^{A} and R^{B} are each C1-C6 alkyl substituted with hydroxyl. In some embodiments, one of R^{A} and R^{B} is C1-C3 alkyl and the other of R^{A} and R^{B} is C1-C3 alkyl substituted with hydroxyl. In some embodiments, one of R^{A} and R^{B} is methyl and the other of R^{A} and R^{B} is C1-C3 alkyl substituted with hydroxyl. In some embodiments, one of R^{A} and R^{B} is methyl and the other of R^{A} and R^{B} is ethyl substituted with hydroxyl (e.g., 2-hydroxy-1-propyl). In some embodiments, R^{A} and R^{B} are each C1-C6 alkyl. In some embodiments, R^{A} and R^{B} are each C1-C3 alkyl. In some embodiments, R^{A} and R^{B} are each methyl.

In some embodiments, R^{4B} is amino or a 4-6 membered heterocyclyl having one nitrogen atom and optionally substituted with 1-2 independently selected R^{G}; wherein R^{G} is selected from fluoro, hydroxyl, and C1-C6 alkyl.

In some embodiments, R^{4B} is

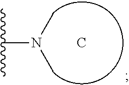

wherein Ring C is azetidinyl, pyrrolidinyl, piperidinyl, piperazinyl, morpholinyl, thiomorpholinyl, each optionally containing 1-2 ═O, and each optionally substituted with 1-2 R^{G} independently selected from fluoro, hydroxyl, trifluoromethyl, amino, cyclopropyl, —CO₂CH₃, and C1-C6 alkyl. In some embodiments, R^{4B} is

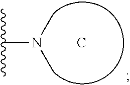

wherein Ring C is azetidinyl, pyrrolidinyl, piperidinyl, piperazinyl, or morpholinyl, each optionally substituted with 1-2 R^{G} independently selected from fluoro, hydroxyl, trifluoromethyl, amino, cyclopropyl, —CO₂CH₃, and C1-C6 alkyl. In some embodiments, R^{4B} is

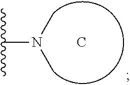

wherein Ring C is azetidinyl, pyrrolidinyl, piperidinyl, piperazinyl, or morpholinyl, each optionally substituted with 1-2 R^{G} independently selected from fluoro, hydroxyl, trifluoromethyl, and C1-C6 alkyl. In some embodiments, R^{4B} is

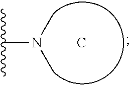

wherein Ring C is azetidinyl, pyrrolidinyl, or piperidinyl, each optionally substituted with 1-2 R^{G} independently selected from fluoro, hydroxyl, and C1-C6 alkyl. In some embodiments, Ring C is azetidinyl.

In some embodiments, Ring C is unsubstituted.

In some embodiments, Ring C is substituted with 1 R^{G}. In some embodiments, R^{G} is fluoro. In some embodiments, R^{G} is cyano. In some embodiments, R^{G} is amino, In some embodiments, R^{G} is hydroxyl. In some embodiments, R^{G} is C1-C3 alkyl. In some embodiments, R^{G} is methyl. In some embodiments, R^{G} is ethyl. In some embodiments, R^{G} is —CO₂CH₃. In some embodiments, R^{G} is methoxy. In some embodiments, R^{G} is methoxy.

In some embodiments, Ring C is substituted with 2 R^{G}. In some embodiments, each R^{G} is fluoro. In some embodiments, each R^{G} is C1-C3 alkyl. In some embodiments, each R^{G} is methyl. In some embodiments, one R^{G} is hydroxyl and the other R^{G} is methyl. In some embodiments, one R^{G} is hydroxyl and the other R^{G} is ethyl. In some embodiments, one R^{G} is amino and the other R^{G}is methyl. In some embodiments, one R^{G} is hydroxyl and the other R^{G} is cyclopropyl. In some embodiments, one R^{G} is fluoro and the other R^{G1} is methyl. In some embodiments, one R^{G} is hydroxyl and the other R^{G} is fluoro. In some embodiments, one R^{G} is hydroxyl and the other R^{G}is trifluoromethyl. In some embodiments, each R^{G} is bonded to the position of Ring C para to the nitrogen that is bonded to Ring A.

In some embodiments,

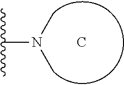

is

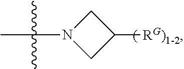

wherein 1 or 2 independently selected R^{G} attach at the 3-position of the azetidine. In some embodiments,

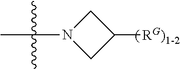

is selected from the group consisting of

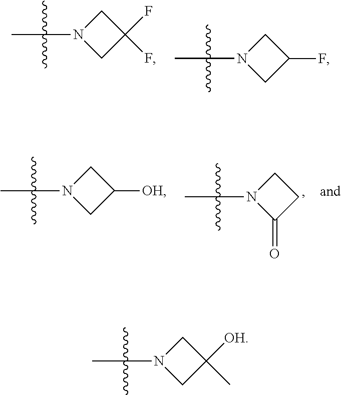

In some embodiments,

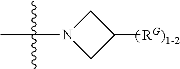

is selected from the group consisting of

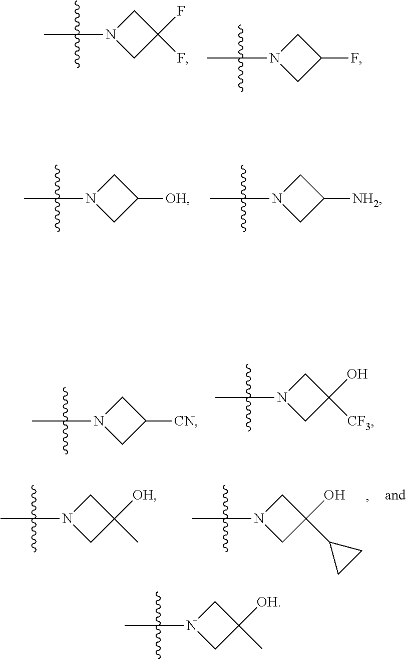

In some embodiments,

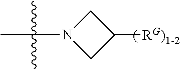

is selected from the group consisting of

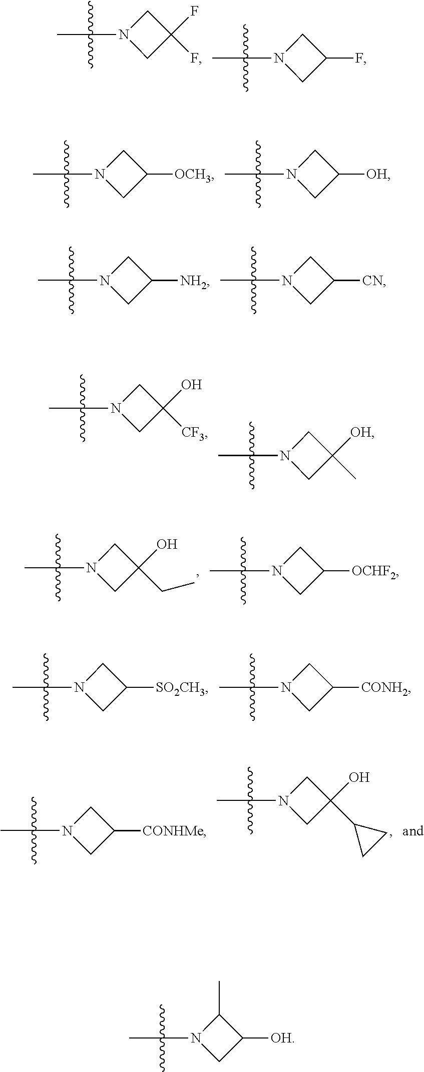

In some embodiments, each R¹ is fluoro; m is 1 or 2; R² is a C1-C6 alkyl; and R³ is a C1-C6 alkyl. In some embodiments, each R¹ is fluoro; m is 1 or 2; R² is methyl; and R³ is selected from methyl, ethyl, isopropyl, or tert-butyl.

In some embodiments, each R¹ is fluoro; m is 1 or 2; R² is a C1-C6 alkyl; and R³ is a C1-C6 haloalkyl. In some embodiments, each R¹ is fluoro; m is 1 or 2; R² is methyl; and R³ is trifluoromethyl.

In some embodiments, m is 2, one R⁴ is halogen, and the other R⁴ is —SO₂(C1-C6 alkyl). In some embodiments, m is 2, one R⁴ is chloro, and the other R⁴ is —SO₂CH₃.

In some embodiments, m is 2, one R⁴ is C1-C6 alkoxy, and the other R⁴ is —C(═O)NR^{C}R^{D}.

In some embodiments, m is 2, one R⁴ is methoxy, and the other R⁴ is —C(O)NHCH₃. In some embodiments, Ring A is a phenyl or a 5-6 membered heteroaryl;
each R⁴ is independently selected from the group consisting of: C1-C3 alkyl, C1-C3 alkoxy, C1-C3 haloalkyl, hydroxyl, cyano, —CO₂H, —NH₂, —C(═O)NH₂, —C(═O)NHMe, —SO₂NH₂, —SO₂NHMe, —SO₂Me, —S(═O)(═NH)Me, —C(═O)Me, 5-6 membered heteroaryl, and unsubstituted 3-6 membered heterocyclyl; and
n is 1 or 2.

In some embodiments, each R¹ is fluoro;
m is 1 or 2;
R² is a C1-C6 alkyl;
R³ is a C1-C6 alkyl;
Ring A is a phenyl or a 5-6 membered heteroaryl;
each R⁴ is independently selected from the group consisting of: C1-C3 alkyl, C1-C3 alkoxy, C1-C3 haloalkyl, hydroxyl, cyano, —NH₂, —C(═O)NH₂, —C(═O)NHMe, —SO₂NH₂, —SO₂NHMe, —SO₂Me, —S(═O)(═NH)Me, —C(═O) Me, 5-6 membered heteroaryl, and unsubstituted 3-6 membered heterocyclyl; and
n is 1 or 2.

In some embodiments, each R¹ is fluoro;
m is 1 or 2;
R² is a C1-C6 alkyl;
R³ is a C1-C6 alkyl;
Ring A is a phenyl or a 5-6 membered heteroaryl;
each R⁴ is independently selected from the group consisting of: C1-C3 alkyl, C1-C3 alkoxy, C1-C3 haloalkyl, hydroxyl, cyano, —NH₂, —C(═O)NH₂, —C(═O)NHMe, —SO₂NH₂, —SO₂NHMe, —SO₂Me, —S(═O)(═NH)Me, —C(═O) Me, 5-6 membered heteroaryl, and 3-6 membered heterocyclyl optionally substituted with 1 or 2 independently selected R^{G}; and
n is 1 or 2.

In some embodiments, each R¹ is fluoro, cyano, or methyl;
m is 1 or 2;
R² is a C1-C3 alkyl;
R³ is a C1-C3 alkyl or C1-C3 haloalkyl;
Ring A is a phenyl or a 5-6 membered heteroaryl;
each R⁴ is independently selected from the group consisting of: C1-C3 alkyl, C1-C3 alkoxy, C1-C3 haloalkyl, hydroxyl, cyano, —NH₂, —C(═O)NH₂, —C(═O)NHMe, —SO₂NH₂, —SO₂NHMe, —SO₂Me, —S(═O)(═NH)Me, —C(═O) Me, 5-6 membered heteroaryl, and unsubstituted 3-6 membered heterocyclyl; and
n is 1 or 2.

In some embodiments, each R¹ is fluoro, cyano, or methyl;
m is 1 or 2;
R² is a C1-C3 alkyl;
R³ is a C1-C3 alkyl or C1-C3 haloalkyl;
Ring A is a phenyl or a 5-6 membered heteroaryl;
each R⁴ is independently selected from the group consisting of: —NHR^{B}, and 4-6 membered heterocyclyl optionally substituted with 1-2 R^{G}; and
n is 1 or 2.

### Non-Limiting Exemplary Compounds

In some embodiments, the compound is selected from the group consisting of the compounds in Examples 1-7 (e.g., Compounds 1-11), or a pharmaceutically acceptable salt thereof.

In some embodiments, the compound is selected from the group consisting of the compounds delineated in Table A, or a pharmaceutically acceptable salt thereof.

TABLE A

| Compound | |
|---|---|
| No. | Structure |
| 1 | 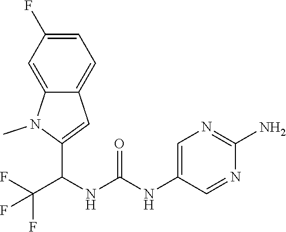 [SMILES: Cn1c(C(NC(=O)Nc2cnc(N)nc2)C(F)(F)F)cc2ccc(F)cc21] |
| 2 | 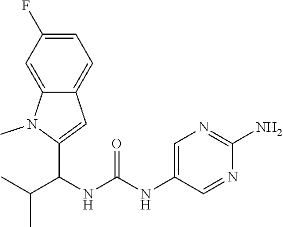 [SMILES: CC(C)C(NC(=O)Nc1cnc(N)nc1)c1cc2ccc(F)cc2n1C] |
| 3 | 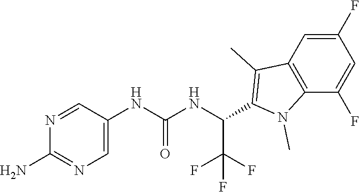 [SMILES: Cc1c([C@@H](NC(=O)Nc2cnc(N)nc2)C(F)(F)F)n(C)c2c(F)cc(F)cc12] |
| 4 | 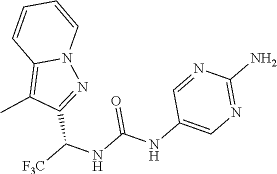 [SMILES: Cc1c([C@H](NC(=O)Nc2cnc(N)nc2)C(F)(F)F)nn2ccccc12] |
| 5 | 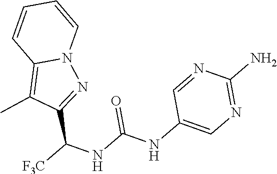 [SMILES: Cc1c([C@@H](NC(=O)Nc2cnc(N)nc2)C(F)(F)F)nn2ccccc12] |
| 6 | 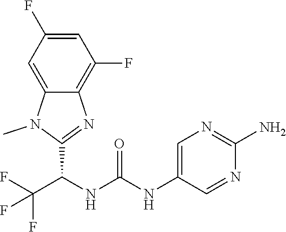 [SMILES: Cn1c([C@H](NC(=O)Nc2cnc(N)nc2)C(F)(F)F)nc2c(F)cc(F)cc21] |
| 7 | 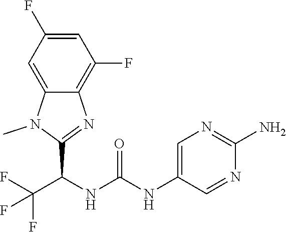 [SMILES: Cn1c([C@@H](NC(=O)Nc2cnc(N)nc2)C(F)(F)F)nc2c(F)cc(F)cc21] |
| 8 | 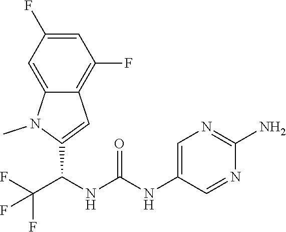 [SMILES: Cn1c([C@H](NC(=O)Nc2cnc(N)nc2)C(F)(F)F)cc2c(F)cc(F)cc21] |
| 9 | 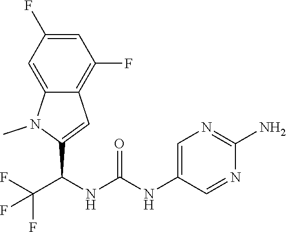 [SMILES: Cn1c([C@@H](NC(=O)Nc2cnc(N)nc2)C(F)(F)F)cc2c(F)cc(F)cc21] |
| 10 | 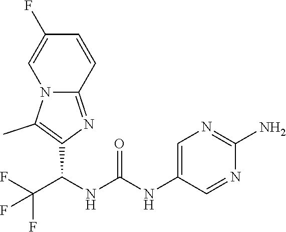 [SMILES: Cc1c([C@H](NC(=O)Nc2cnc(N)nc2)C(F)(F)F)nc2ccc(F)cn12] |
| 11 | 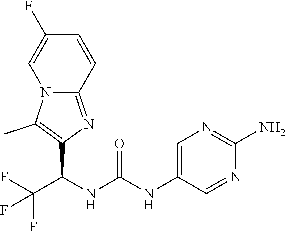 [SMILES: Cc1c([C@@H](NC(=O)Nc2cnc(N)nc2)C(F)(F)F)nc2ccc(F)cn12] |

### Pharmaceutical Compositions

Some embodiments provide a pharmaceutical composition comprising a compound of Formula (I), or a pharmaceutically acceptable salt thereof, and one or more pharmaceutically acceptable excipients.

### Methods of Treatment

### Indications

Provided herein are methods for inhibiting phosphatidylinositol 4,5-bisphosphate 3-kinase isoform alpha (PI3Kα), encoded by PIK3CA gene. For example, provided herein are inhibitors of PI3Kα useful for treating or preventing diseases or disorders associated with dysregulation of a PIK3CA gene, a PI3Kα protein, or the expression or activity or level of any of the same (i.e., a PI3Kα-associated disease or disorder), such as PIK3CA-related overgrowth syndromes ((PROS), see, e.g., Venot, et al., Nature, 558, 540-546 (2018)), brain disorders (e.g., as macrocephaly-capillary malformation (MCAP) and hemimegalencephaly), congenital lipomatous (e.g., overgrowth of vascular malformations), epidermal nevi and skeletal/spinal anomalies (e.g., CLOVES syndrome) and fibroadipose hyperplasia (FH), or cancer (e.g., PI3Kα-associated cancer).

A “PI3Kα inhibitor” as used herein includes any compound exhibiting PI3Kα inactivation activity (e.g., inhibiting or decreasing). In some embodiments, a PI3Kα inhibitor can be selective for a PI3Kα having one or more mutations.

The ability of test compounds to act as inhibitors of PI3Kα may be demonstrated by assays known in the art. The activity of the compounds and compositions provided herein as PI3Kα inhibitors can be assayed in vitro, in vivo, or in a cell line. In vitro assays include assays that determine inhibition of the kinase. Alternate in vitro assays quantitate the ability of the inhibitor to bind to the protein kinase and can be measured either by radio labeling the compound prior to binding, isolating the compound/kinase complex and determining the amount of radio label bound, or by running a competition experiment where new compounds are incubated with the kinase bound to known radio ligands.

Potency of a PI3Kα inhibitor as provided herein can be determined by EC₅₀ value. A compound with a lower EC₅₀ value, as determined under substantially similar conditions, is a more potent inhibitor relative to a compound with a higher EC₅₀ value. In some embodiments, the substantially similar conditions comprise determining a PI3Kα-dependent phosphorylation level, in vitro or in vivo (e.g., in tumor cells, A594 cells, U2OS cells, A431 cells, Ba/F3 cells, or 3T3 cells expressing a wild type PI3Kα, a mutant PI3Kα, or a fragment of any thereof).

Potency of a PI3Kα inhibitor as provided herein can also be determined by IC₅₀ value. A compound with a lower IC₅₀ value, as determined under substantially similar conditions, is a more potent inhibitor relative to a compound with a higher IC₅₀ value. In some embodiments, the substantially similar conditions comprise determining a PI3Kα-dependent phosphorylation level, in vitro or in vivo (e.g., in tumor cells, SKOV3, T47D, CAL33, BT20, HSC2, OAW42, NCI, HCC1954, NCIH1048, Detroit562, A594 cells, U2OS cells, A431 cells, A594 cells, U2OS cells, Ba/F3 cells, or 3T3 cells expressing a wild type PI3Kα, a mutant PI3Kα, or a fragment of any thereof).

The selectivity between wild type PI3Kα and PI3Kα containing one or more mutations as described herein can also be measured using in vitro assays such as surface plasmon resonance and fluorence-based binding assays, and cellular assays such as the levels of pAKT, abiomarker of PI3Kα activity, or proliferation assays where cell proliferation is dependent on mutant PI3Kα kinase activity.

In some embodiments, the compounds provided herein can exhibit potent and selective inhibition of PI3Kα. For example, the compounds provided herein can bind to the helical phosphatidylinositol kinase homology domain catalytic domain of PI3Kα. In some embodiments, the compounds provided herein can exhibit nanomolar potency against a PI3Kα kinase including one or more mutations, for example, the mutations in Tables 1 and 2.

In some embodiments, the compounds provided herein can exhibit potent and selective inhibition of mutant PI3Kα. For example, the compounds provided herein can bind to an alloseric site in the kinase domain. In some embodiments, the compounds provided herein can exhibit nanomolar potency against a PI3Kα protein including an activating mutation, with minimal activity against related kinases (e.g., wild type PI3Kα). Inhibition of wild type PI3Kα can cause undesireable side effects (e.g., hyperglycemia and skin rashes) that can impact quality of life and compliance. In some cases, the inhibititon of wild type PI3Kα can lead to dose limiting toxicities. See, e.g., Hanker, et al., Cancer Disc. 2019, 9, 4, 482-491. Mutant-selective inhibitors may reduce the risk of such dose limiting toxicities, including hyperglycemia, observed with inhibitors of wild type PI3Kα.

In some embodiments, the compounds of Formula (I), or a pharmaceutically acceptable salt thereof, can selectively target PI3Kα. For example, a compound of Formula (I), or a pharmaceutically acceptable salt thereof, can selectively target PI3Kα over another kinase or non-kinase target.

In some embodiments, a compound of Formula (I), or a pharmaceutically acceptable salt thereof, can exhibit greater inhibition of PI3Kα containing one or more mutations as described herein (e.g., one or more mutations as described in Table 1 or Table 2) relative to inhibition of wild type PI3Kα. In some embodiments, a compound of Formula (I), or a pharmaceutically acceptable salt thereof can exhibit at least 2-fold, 3-fold, 5-fold, 10-fold, 25-fold, 50-fold or 100-fold greater inhibition of PI3Kα containing one or more mutations as described herein relative to inhibition of wild type PI3Kα. In some embodiments, a compound of Formula (I), or a pharmaceutically acceptable salt thereof, can exhibit up to 1000-fold greater inhibition of PI3Kα containing one or more mutations as described herein relative to inhibition of wild type PI3Kα. In some embodiments, a compound of Formula (I), or a pharmaceutically acceptable salt thereof, can exhibit up to 10000-fold greater inhibition of PI3Kα having a combination of mutations described herein relative to inhibition of wild type PI3Kα.

In some embodiments, a compound of Formula (I), or a pharmaceutically acceptable salt thereof, can exhibit from about 2-fold to about 10-fold greater inhibition of PI3Kα containing one or more mutations as described herein relative to inhibition of wild type PI3Kα. In some embodiments, a compound of Formula (I), or a pharmaceutically acceptable salt thereof, can exhibit from about 10-fold to about 100-fold greater inhibition of PI3Kα containing one or more mutations as described herein relative to inhibition of wild type PI3Kα. In some embodiments, a compound of Formula (I), or a pharmaceutically acceptable salt thereof, can exhibit from about 100-fold to about 1000-fold greater inhibition of PI3Kα containing one or more mutations as described herein relative to inhibition of wild type PI3Kα. In some embodiments, a compound of Formula (I), or a pharmaceutically acceptable salt thereof, can exhibit from about 1000-fold to about 10000-fold greater inhibition of PI3Kα containing one or more mutations as described herein relative to inhibition of wild type PI3Kα.

Compounds of Formula (I), or pharmaceutically acceptable salts thereof, are useful for treating diseases and disorders which can be treated with a PI3Kα inhibitor, such as PI3Kα-associated diseases and disorders, e.g., PIK3CA-related overgrowth syndromes (PROS) and proliferative disorders such as cancers, including hematological cancers and solid tumors (e.g., advanced or metastatic solid tumors).

In some embodiments, the subject has been identified or diagnosed as having a cancer with a dysregulation of a PIK3CA gene, a PI3Kα protein, or expression or activity, or level of any of the same (a PI3Kα-associated cancer) (e.g., as determined using a regulatory agency-approved, e.g., FDA-approved, assay or kit). In some embodiments, the subject has a tumor that is positive for a dysregulation of a PIK3CA gene, a PI3Kα protein, or expression or activity, or level of any of the same (e.g., as determined using a regulatory agency-approved assay or kit). For example, the subject has a tumor that is positive for a mutation as described in Table 1 or Table 2. The subject can be a subject with a tumor(s) that is positive for a dysregulation of a PIK3CA gene, a PI3Kα protein, or expression or activity, or level of any of the same (e.g., identified as positive using a regulatory agency-approved, e.g., FDA-approved, assay or kit). The subject can be a subject whose tumors have a dysregulation of a PIK3CA gene, a PI3Kα protein, or expression or activity, or a level of the same (e.g., where the tumor is identified as such using a regulatory agency-approved, e.g., FDA-approved, kit or assay). In some embodiments, the subject is suspected of having a PI3Kα-associated cancer. In some embodiments, the subject has a clinical record indicating that the subject has a tumor that has a dysregulation of a PIK3CA gene, a PI3Kα protein, or expression or activity, or level of any of the same (and optionally the clinical record indicates that the subject should be treated with any of the compositions provided herein).

In some embodiments, the subject is a pediatric subject.

The term “pediatric subject” as used herein refers to a subject under the age of 21 years at the time of diagnosis or treatment. The term “pediatric” can be further be divided into various subpopulations including: neonates (from birth through the first month of life); infants (1 month up to two years of age); children (two years of age up to 12 years of age); and adolescents (12 years of age through 21 years of age (up to, but not including, the twenty-second birthday)). Berhman R E, Kliegman R, Arvin A M, Nelson W E. Nelson Textbook of Pediatrics, 15th Ed. Philadelphia: W.B. Saunders Company, 1996; Rudolph A M, et al. Rudolph's Pediatrics, 21st Ed. New York: McGraw-Hill, 2002; and Avery M D, First L R. Pediatric Medicine, 2nd Ed. Baltimore: Williams & Wilkins; 1994. In some embodiments, a pediatric subject is from birth through the first 28 days of life, from 29 days of age to less than two years of age, from two years of age to less than 12 years of age, or 12 years of age through 21 years of age (up to, but not including, the twenty-second birthday). In some embodiments, a pediatric subject is from birth through the first 28 days of life, from 29 days of age to less than 1 year of age, from one month of age to less than four months of age, from three months of age to less than seven months of age, from six months of age to less than 1 year of age, from 1 year of age to less than 2 years of age, from 2 years of age to less than 3 years of age, from 2 years of age to less than seven years of age, from 3 years of age to less than 5 years of age, from 5 years of age to less than 10 years of age, from 6 years of age to less than 13 years of age, from 10 years of age to less than 15 years of age, or from 15 years of age to less than 22 years of age.

In certain embodiments, compounds of Formula (I), or pharmaceutically acceptable salts thereof, are useful for preventing diseases and disorders as defined herein (for example, PIK3CA-related overgrowth syndromes (PROS) and cancer). The term “preventing” as used herein means to delay the onset, recurrence or spread, in whole or in part, of the disease or condition as described herein, or a symptom thereof.

The term “PI3Kα-associated disease or disorder” as used herein refers to diseases or disorders associated with or having a dysregulation of a PIK3CA gene, a PI3Kα protein, or the expression or activity or level of any (e.g., one or more) of the same (e.g., any of the types of dysregulation of a PIK3CA gene, or a PI3Kα protein, or the expression or activity or level of any of the same described herein). Non-limiting examples of a PI3Kα-associated disease or disorder include, for example, PIK3CA-related overgrowth syndromes (PROS), brain disorders (e.g., as macrocephaly-capillary malformation (MCAP) and hemimegalencephaly), congenital lipomatous (e.g., overgrowth of vascular malformations), epidermal nevi and skeletal/spinal anomalies (e.g., CLOVES syndrome) and fibroadipose hyperplasia (FH), or cancer (e.g., PI3Kα-associated cancer).

The term “PI3Kα-associated cancer” as used herein refers to cancers associated with or having a dysregulation of a PIK3CA gene, a PI3Kα protein, or expression or activity, or level of any of the same. Non-limiting examples of PI3Kα-associated cancer are described herein.

The phrase “dysregulation of a PIK3CA gene, a PI3Kα protein, or the expression or activity or level of any of the same” refers to a genetic mutation (e.g., a mutation in a PIK3CA gene that results in the expression of a PI3Kα that includes a deletion of at least one amino acid as compared to a wild type PI3Kα, a mutation in a PIK3CA gene that results in the expression of PI3Kα with one or more point mutations as compared to a wild type PI3Kα, a mutation in a PIK3CA gene that results in the expression of PI3Kα with at least one inserted amino acid as compared to a wild type PI3Kα, a gene duplication that results in an increased level of PI3Kα in a cell, or a mutation in a regulatory sequence (e.g., a promoter and/or enhancer) that results in an increased level of PI3Kα in a cell), an alternative spliced version of PI3Kα mRNA that results in PI3Kα having a deletion of at least one amino acid in the PI3Kα as compared to the wild type PI3Kα), or increased expression (e.g., increased levels) of a wild type PI3Kα in a mammalian cell due to aberrant cell signaling and/or dysregulated autocrine/paracrine signaling (e.g., as compared to a control non-cancerous cell). As another example, a dysregulation of a PIK3CA gene, a PI3Kα protein, or expression or activity, or level of any of the same, can be a mutation in a PIK3CA gene that encodes a PI3Kα that is constitutively active or has increased activity as compared to a protein encoded by a PIK3CA gene that does not include the mutation. Non-limiting examples of PI3Kα point mutations/substitutions/insertions/deletions are described in Table 1 and Table 2.

The term “activating mutation” in reference to PI3Kα describes a mutation in a PIK3CA gene that results in the expression of PI3Kα that has an increased kinase activity, e.g., as compared to a wild type PI3Kα, e.g., when assayed under identical conditions. For example, an activating mutation can be a mutation in a PIK3CA gene that results in the expression of a PI3Kα that has one or more (e.g., two, three, four, five, six, seven, eight, nine, or ten) amino acid substitutions (e.g., any combination of any of the amino acid substitutions described herein) that has increased kinase activity, e.g., as compared to a wild type a PI3Kα, e.g., when assayed under identical conditions. In another example, an activating mutation can be a mutation in a PIK3CA that results in the expression of a PI3Kα that has one or more (e.g., two, three, four, five, six, seven, eight, nine, or ten) amino acids deleted, e.g., as compared to a wild type PI3Kα, e.g., when assayed under identical conditions. In another example, an activating mutation can be a mutation in a PIK3CA gene that results in the expression of a PI3Kα that has at least one (e.g., at least 2, at least 3, at least 4, at least 5, at least 6, at least 7, at least 8, at least 9, at least 10, at least 12, at least 14, at least 16, at least 18, or at least 20) amino acid inserted as compared to a wild type PI3Kα, e.g., the exemplary wild type PI3Kα described herein, e.g., when assayed under identical conditions. Additional examples of activating mutations are known in the art.

The term “wild type” or “wild-type” describes a nucleic acid (e.g., a PIK3CA gene or a PI3Kα mRNA) or protein (e.g., a PI3Kα) sequence that is typically found in a subject that does not have a disease or disorder related to the reference nucleic acid or protein.

The term “wild type PI3Kα” or “wild-type PI3Kα ” describes a normal PI3Kα nucleic acid (e.g., a PIK3CA or PI3Kα mRNA) or protein that is found in a subject that does not have a PI3Kα-associated disease, e.g., a PI3Kα-associated cancer (and optionally also does not have an increased risk of developing a PI3Kα-associated disease and/or is not suspected of having a PI3Kα-associated disease), or is found in a cell or tissue from a subject that does not have a PI3Kα-associated disease, e.g., a PI3Kα-associated cancer (and optionally also does not have an increased risk of developing a PI3Kα-associated disease and/or is not suspected of having a PI3Kα-associated disease).

Provided herein is a method of treating cancer (e.g., a PI3Kα-associated cancer) in a subject in need of such treatment, the method comprising administering to the subject a therapeutically effective amount of a compound of Formula (I), or a pharmaceutically acceptable salt thereof, or a pharmaceutical composition thereof. For example, provided herein are methods for treating PI3Kα-associated cancer in a subject in need of such treatment, the method comprising a) detecting a dysregulation of PIK3CA gene, a PI3Kα protein, or the expression or activity or level of any of the same in a sample from the subject; and b) administering a therapeutically effective amount of a compound of Formula (I), or a pharmaceutically acceptable salt thereof. In some embodiments, the dysregulation of a PIK3CA gene, a PI3Kα protein, or the expression or activity or level of any of the same includes one or more a PI3Kα protein substitutions/point mutations/insertions. Non-limiting examples of PI3Kα protein substitutions/insertions/deletions are described in Table 1 and Table 2.

In some embodiments, the PI3Kα protein substitution/insertion/deletion is selected from the group consisting of E542A, E542G, E542K, E542Q, E542V, E545A, E545D, E545G, E545K, E545Q, M10431, M1043L, M1043T, M1043V, H1047L, H1047Q, H1047R, H1047Y, G1049R, and combinations thereof. In some embodiments, the PI3Kα protein substitution/insertion/deletion is H1047X, where X is any amino acid.

In some embodiments of any of the methods or uses described herein, the cancer (e.g., PI3Kα-associated cancer) is selected from a hematological cancer and a solid tumor.

In some embodiments of any of the methods or uses described herein, the cancer (e.g., PI3Kα-associated cancer) is selected from breast cancer (including both HER2⁺ and HER2⁻ breast cancer, ER⁺ breast cancer, and triple negative breast cancer), endometrial cancer, lung cancer (including adenocarcinoma lung cancer and squamous cell lung carcinoma), esophageal squamous cell carcinoma, ovarian cancer, colorectal cancer, esophagastric adenocarcinoma, bladder cancer, head and neck cancer (including head and neck squamous cell cancers such as oropharyngeal squamous cell carcinoma), thyroid cancer, glioma, cervical cancer, lymphangioma, meningioma, melanoma (including uveal melanoma), kidney cancer, pancreatic neuroendocine neoplasms (pNETs), stomach cancer, esophageal cancer, acute myeloid leukemia, relapsed and refractory multiple myeloma, and pancreatic cancer.

In some embodiments of any of the methods or uses described herein, the cancer (e.g., PI3Kα-associated cancer) is selected from breast cancer (including both HER2⁺ and HER2⁻ breast cancer, ER⁺ breast cancer, and triple negative breast cancer), colon cancer, rectal cancer, colorectal cancer, ovarian cancer, lymphangioma, meningioma, head and neck squamous cell cancer (including oropharyngeal squamous cell carcinoma), melanoma (including uveal melanoma), kidney cancer, pancreatic neuroendocine neoplasms (pNETs), stomach cancer, esophageal cancer, acute myeloid leukemia, relapsed and refractory multiple myeloma, pancreatic cancer, lung cancer (including adenocarcinoma lung cancer and squamous cell lung carcinoma), and endometrial cancer.

In some embodiments of any of the methods or uses described herein, the cancer (e.g., PI3Kα-associated cancer) is selected from breast cancer, lung cancer, endometrial cancer, esophageal squamous cell carcinoma, ovarian cancer, colorectal cancer, esophagastric adenocarcinoma, bladder cancer, head and neck cancer, thyroid cancer, glioma, and cervical cancer.

In some embodiments of any of the methods or uses described herein, the PI3Kα-associated cancer is breast cancer. In some embodiments of any of the methods or uses described herein, the PI3Kα-associated cancer is colorectal cancer. In some embodiments of any of the methods or uses described herein, the PI3K-associated cancer is endometrial cancer. In some embodiments of any of the methods or uses described herein, the PI3Kα-associated cancer is lung cancer.

In some embodiments of any of the methods or uses described herein, the PI3Kα-associated cancer is selected from the cancers described in Table 1 and Table 2.

TABLE 1
PI3Kα Protein Amino Acid Substitutions/Insertions/Deletions^{A}

| | Non-Limiting | |
|---|---|---|
| Amino Acid | Exemplary | Non-Limiting Exemplary PI3Kα |
| Position | Mutations | Associated Cancer(s) |
| 1 | M1 (Translation | Astrocytoma |
| | Start Site) | Glioblastoma Multiforme |
| 4 | R4* (Nonsense | Glioblastoma Multiforme |
| | Mutation) | |
| 9 | E9G | Stomach Adenocarcinoma |
| 10 | L10_M16del | Glioblastoma Multiforme |
| 11 | W11L, W11S, | Lung Adenocarcinoma, |
| | W11_P18del (In | Oligodendroglioma, |
| | Frame Deletion) | Uterine Endometrioid Carcinoma |
| 12 | G12D | Uterine Endometrioid Carcinoma |
| 13 | I13T | Colon Adenocarcinoma |
| 19 | R19I | Uterine Endometrioid Carcinoma |
| 27 | P27T | Hepatocellular Carcinoma |
| 36 | C36Y | Uterine Endometrioid Carcinoma |
| 38 | R38C, | Uterine Endometrioid Carcinoma |
| | R38H, | Papillary Renal Cell Carcinoma |
| | R38L, | Papillary Stomach Adenocarcinoma |
| | R38S | Mucinous Adenocarcinoma of |
| | | the Colon and Rectum |
| | | Glioblastoma Multiforme |
| | | Cervical Squamous Cell Carcinoma |
| | | Hepatocellular Carcinoma |
| | | Uterine Endometrioid Carcinoma |
| | | Diffuse Type Stomach |
| | | Adenocarcinoma |
| | | Lung Squamous Cell Carcinoma |
| | | Uterine Endometrioid Carcinoma |
| 39 | E39G, | Uterine Endometrioid Carcinoma |
| | E39K | Glioblastoma Multiforme |
| 57 | P57L | Cutaneous Melanoma |
| 65 | E65K | Lung Squamous Cell Carcinoma |
| 66 | S66C | Bladder Urothelial Carcinoma |
| 69 | I69N | Colon Adenocarcinoma |
| 71 | V71I | Head and Neck Squamous Cell |
| | | Carcinoma |
| 75 | Q75E | Bladder Urothelial Carcinoma |
| | | Cervical Squamous Cell Carcinoma |
| | | Head and Neck Squamous Cell |
| | | Carcinoma |
| 78 | E78* (nonsense | Lung Squamous Cell Carcinoma |
| | mutation) | |
| 80 | E80K | Uterine Mixed Endometrial |
| | | Carcinoma |
| 81 | E81* (nonsense | Colon Adenocarcinoma |
| | mutation), E81del | Glioblastoma Multiforme |
| | (in frame | Colon Adenocarcinoma |
| | deletion), E81K | Uterine Serous Carcinoma/Uterine |
| | | Papillary Serous |
| | | Carcinoma |
| | | Glioblastoma Multiforme |
| | | Uterine Endometrioid Carcinoma |
| | | Lung Squamous Cell Carcinoma |
| | | Mucinous Adenocarcinoma of |
| | | the Colon and Rectum |
| | | Breast Invasive Ductal Carcinoma |
| | | Cervical Squamous Cell Carcinoma |
| | | Head and Neck Squamous Cell |
| | | Carcinoma |
| 83 | F83L, F83S | Breast Invasive Lobular Carcinoma |
| 84 | D84H | Lung Adenocarcinoma |
| 86 | T86S | Hepatocellular Carcinoma |
| 87 | R87T | Lung Adenocarcinoma |
| 88 | R88Q | Breast Invasive Ductal Carcinoma |
| | | Rectal Adenocarcinoma |
| | | Colon Adenocarcinoma |
| | | Prostate Adenocarcinoma |
| | | Cervical Squamous Cell Carcinoma |
| | | Uterine Carcinosarcoma/Uterine |
| | | Malignant Mixed |
| | | Mullerian Tumor |
| | | Tubular Stomach Adenocarcinoma |
| | | Oligodendroglioma |
| | | Mucinous Stomach Adenocarcinoma |
| | | Glioblastoma Multiforme |
| | | Stomach Adenocarcinoma |
| | | Uterine Endometrioid Carcinoma |
| | | Uterine Mixed Endometrial |
| | | Carcinoma |
| | | Head and Neck Squamous Cell |
| | | Carcinoma |
| | | Mucinous Adenocarcinoma of |
| | | the Colon and Rectum |
| | | Breast Invasive Lobular Carcinoma |
| | | Intestinal Type Stomach |
| | | Adenocarcinoma |
| | | Bladder Urothelial Carcinoma |
| 90 | C90G, C90R, | Glioblastoma Multiforme |
| | C90Y | Cervical Squamous Cell Carcinoma |
| 93 | R93P, R93Q, | Mucinous Adenocarcinoma of |
| | R93W | the Colon and Rectum |
| | | Stomach Adenocarcinoma |
| | | Glioblastoma Multiforme |
| | | Uterine Endometrioid Carcinoma |
| | | Tubular Stomach Adenocarcinoma |
| | | Mucinous Stomach Adenocarcinoma |
| | | Bladder Urothelial Carcinoma |
| | | Cervical Squamous Cell Carcinoma |
| | | Colon Adenocarcinoma |
| 102 | I102del | Uterine Endometrioid Carcinoma |
| 103 | E103G, | Glioblastoma Multiforme |
| | E103_G106delins | Breast Invasive Ductal Carcinoma |
| | D (In Frame | |
| | Deletion), | |
| | E103_P104del (In | |
| | Frame Deletion) | |
| 104 | P104L, P104R, | Breast Invasive Ductal Carcinoma |
| | P104T | Head and Neck Squamous Cell |
| | | Carcinoma |
| | | Lung Adenocarcinoma |
| | | Colon Adenocarcinoma |
| | | Uterine Serous Carcinoma/Uterine |
| | | Papillary Serous |
| | | Carcinoma |
| 105 | V105del, | Uterine Endometrioid Carcinoma |
| | V105_R108del | Breast Invasive Ductal Carcinoma |
| 106 | G106D, G106R, | Uterine Mixed Endometrial |
| | G106S, G106V, | Carcinoma |
| | G106_R108del | Breast Invasive Ductal Carcinoma |
| | (In Frame | Mucinous Adenocarcinoma of the |
| | Deletion), | Colon and Rectum |
| | G106_N107del | Mucinous Carcinoma |
| | (In Frame | Oligodendroglioma |
| | Deletion) | Uterine Carcinosarcoma/Uterine |
| | | Malignant Mixed |
| | | Mullerian Tumor |
| | | Uterine Serous Carcinoma/Uterine |
| | | Papillary Serous |
| | | Carcinoma |
| | | Uterine Endometrioid Carcinoma |
| | | Rectal Adenocarcinoma |
| | | Lung Squamous Cell Carcinoma |
| | | Cervical Squamous Cell Carcinoma |
| | | Tubular Stomach Adenocarcinoma |
| | | Uterine Endometrioid Carcinoma |
| 107 | N107S | Uterine Endometrioid Carcinoma |
| | | Lung Adenocarcinoma |
| 108 | R108C, R108H, | Prostate Adenocarcinoma |
| | R108L | Uterine Endometrioid Carcinoma |
| | | Glioblastoma Multiforme |
| | | Uterine Carcinosarcoma/Uterine |
| | | Malignant Mixed |
| | | Mullerian Tumor |
| | | Mucinous Adenocarcinoma of the |
| | | Colon and Rectum |
| | | Tubular Stomach Adenocarcinoma |
| | | Colon Adenocarcinoma |
| | | Uterine Serous Carcinoma/Uterine |
| | | Papillary Serous |
| | | Carcinoma |
| | | Uterine Mixed Endometrial |
| | | Carcinoma |
| | | Breast Invasive Ductal Carcinoma |
| | | Lung Squamous Cell Carcinoma |
| 109 | E109_I112delins | Breast Invasive Ductal Carcinoma |
| | D (In Frame | |
| | Deletion) | |
| 110 | E110del | Uterine Endometrioid Carcinoma |
| | | Oligodendroglioma |
| | | Breast Invasive Lobular Carcinoma |
| | | Breast Invasive Ductal Carcinoma |
| | | Uterine Mixed Endometrial |
| | | Carcinoma |
| | | Colon Adenocarcinoma |
| | | Head and Neck Squamous Cell |
| | | Carcinoma |
| | | Lung Adenocarcinoma |
| | | Papillary Thyroid Cancer |
| 111 | K111del, K111E, | Uterine Endometrioid Carcinoma |
| | K111N, K111R, | Breast Invasive Ductal Carcinoma |
| | K111_L113del | Oligodendroglioma |
| | (In Frame | Head and Neck Squamous Cell |
| | Deletion) | Carcinoma |
| | | Colon Adenocarcinoma |
| | | Intestinal Type Stomach |
| | | Adenocarcinoma |
| | | Stomach Adenocarcinoma |
| | | Uterine Endometrioid Carcinoma |
| | | Lung Adenocarcinoma |
| | | Esophageal Adenocarcinoma |
| | | Lung Squamous Cell Carcinoma |
| | | Glioblastoma Multiforme |
| 113 | L113del | Uterine Endometrioid Carcinoma |
| 115 | R115L, R115P | Serous Ovarian Cancer |
| | | Bladder Urothelial Carcinoma |
| | | Uterine Carcinosarcoma/Uterine |
| | | Malignant Mixed |
| | | Mullerian Tumor |
| | | Uterine Endometrioid Carcinoma |
| | | Uterine Serous Carcinoma/Uterine |
| | | Papillary Serous |
| | | Carcinoma |
| | | Rectal Adenocarcinoma |
| | | Cervical Squamous Cell Carcinoma |
| 116 | E116K | Rectal Adenocarcinoma |
| 118 | G118D | Glioblastoma Multiforme |
| | | Breast Invasive Ductal Carcinoma |
| | | Uterine Endometrioid Carcinoma |
| | | Bladder Urothelial Carcinoma |
| | | Oligodendroglioma |
| | | Esophageal Adenocarcinoma |
| | | Astrocytoma |
| | | Lung Squamous Cell Carcinoma |
| | | Breast Invasive Lobular Carcinoma |
| | | Uterine Serous Carcinoma/Uterine |
| | | Papillary Serous |
| | | Carcinoma |
| | | Mucinous Carcinoma |
| | | Uterine Mixed Endometrial |
| | | Carcinoma |
| | | Stomach Adenocarcinoma |
| | | Pancreatic Adenocarcinoma |
| | | Papillary Thyroid Cancer |
| | | Rectal Adenocarcinoma |
| 123 | M123I | Lung Adenocarcinoma |
| 124 | P124A | Lung Adenocarcinoma |
| 151 | V151M | Bladder Urothelial Carcinoma |
| | | Astrocytoma |
| 165 | Y165H | Uterine Mixed Endometrial |
| | | Carcinoma |
| 170 | N170S | Uterine Endometrioid Carcinoma |
| 182 | Y182H | Stomach Adenocarcinoma |
| 213 | H213N | Cutaneous Melanoma |
| 224 | A224S | Colon Adenocarcinoma |
| 239 | L239R | Colon Adenocarcinoma |
| 258 | D258N | Rectal Adenocarcinoma |
| 262 | L262I | Cutaneous Melanoma |
| 266 | P266T | Uterine Endometrioid Carcinoma |
| 267 | L267M | Cutaneous Melanoma |
| 272 | Y272* (Nonsense | Renal Clear Cell Carcinoma |
| | Mutation) | |
| 274 | R274K | Bladder Urothelial Carcinoma |
| 279 | L279I | Uterine Endometrioid Carcinoma |
| 282 | M282V | Uterine Endometrioid Carcinoma |
| 292 | S292I | Glioblastoma Multiforme |
| 296 | Q296E | Lung Adenocarcinoma |
| 300 | D300V | Lung Squamous Cell Carcinoma |
| 310 | R310C | Uterine Endometrioid Carcinoma |
| 322 | T322A | Uterine Endometrioid Carcinoma |
| 335 | R335G | Head and Neck Squamous Cell |
| | | Carcinoma |
| 337 | K337N | Rectal Adenocarcinoma |
| 339 | L339I | Cervical Squamous Cell Carcinoma |
| | | Uterine Endometrioid Carcinoma |
| 342 | T342S | Lung Adenocarcinoma |
| 344 | V344A, V344G, | Uterine Endometrioid Carcinoma |
| | V344M | Mucinous Adenocarcinoma of |
| | | the Colon and Rectum |
| | | Colon Adenocarcinoma |
| | | Cervical Squamous Cell Carcinoma |
| | | Rectal Adenocarcinoma |
| | | Head and Neck Squamous Cell |
| | | Carcinoma |
| | | Breast Invasive Carcinoma |
| | | (NOS) |
| | | Uterine Mixed Endometrial |
| | | Carcinoma |
| | | Glioblastoma Multiforme |
| 345 | N345H, N345I, | Breast Invasive Lobular Carcinoma |
| | N345K, N345T, | Uterine Carcinosarcoma/Uterine |
| | N345Y | Malignant Mixed |
| | | Mullerian Tumor |
| | | Uterine Serous Carcinoma/Uterine |
| | | Papillary Serous |
| | | Carcinoma |
| | | Uterine Endometrioid Carcinoma |
| | | Breast Invasive Carcinoma (NOS) |
| | | Lung Adenocarcinoma |
| | | Mucinous Adenocarcinoma of |
| | | the Colon and Rectum |
| | | Bladder Urothelial Carcinoma |
| | | Colon Adenocarcinoma |
| | | Leiomyosarcoma |
| | | Glioblastoma Multiforme |
| | | Uterine Endometrioid Carcinoma |
| | | Seminoma |
| | | Tubular Stomach Adenocarcinoma |
| | | Breast Invasive Ductal Carcinoma |
| | | Head and Neck Squamous Cell |
| | | Carcinoma |
| | | Stomach Adenocarcinoma |
| | | Diffuse Type Stomach |
| | | Adenocarcinoma |
| | | Prostate Adenocarcinoma |
| | | Breast Invasive Ductal Carcinoma |
| | | Oligodendroglioma |
| 350 | D350G, D350N | Lung Squamous Cell Carcinoma |
| | | Breast Invasive Ductal Carcinoma |
| | | Uterine Endometrioid Carcinoma |
| | | Colon Adenocarcinoma |
| | | Uterine Endometrioid Carcinoma |
| | | Mucinous Stomach Adenocarcinoma |
| | | Lung Adenocarcinoma |
| | | Breast Invasive Lobular Carcinoma |
| 351 | I351T | Uterine Endometrioid Carcinoma |
| 357 | R357Q | Uterine Endometrioid Carcinoma |
| | | Mucinous Adenocarcinoma of |
| | | the Colon and Rectum |
| | | Colon Adenocarcinoma |
| 359 | G359R | Uterine Endometrioid Carcinoma |
| 363 | G363A | Head and Neck Squamous Cell |
| | | Carcinoma |
| 364 | G364R | Uterine Mixed Endometrial |
| | | Carcinoma |
| | | Intestinal Type Stomach |
| | | Adenocarcinoma |
| | | Colon Adenocarcinoma |
| 365 | E365K, E365V | Uterine Endometrioid Carcinoma |
| | | Bladder Urothelial Carcinoma |
| | | Mucinous Adenocarcinoma of |
| | | the Colon and Rectum |
| | | Diffuse Type Stomach |
| | | Adenocarcinoma |
| | | Breast Invasive Ductal Carcinoma |
| | | Head and Neck Squamous Cell |
| | | Carcinoma |
| 366 | P366R | Breast Invasive Ductal Carcinoma |
| 378 | C378F, C378R, | Uterine Serous Carcinoma/Uterine |
| | C378Y | Papillary Serous |
| | | Carcinoma |
| | | Uterine Endometrioid Carcinoma |
| | | Oligodendroglioma |
| 379 | S379T | Cutaneous Melanoma |
| 380 | N380S | Diffuse Type Stomach |
| | | Adenocarcinoma |
| 390 | D390N | Lung Adenocarcinoma |
| 392 | Y392H | Uterine Endometrioid Carcinoma |
| 398 | R398H | Breast Invasive Ductal Carcinoma |
| 399 | A399T | Cervical Squamous Cell Carcinoma |
| 401 | R401Q | Uterine Endometrioid Carcinoma |
| 405 | S405F | Intestinal Type Stomach |
| | | Adenocarcinoma |
| 406 | I406V | Uterine Mixed Endometrial |
| | | Carcinoma |
| 412 | R412Q | Stomach Adenocarcinoma |
| 417 | E417K | Bladder Urothelial Carcinoma |
| 418 | E418K | Uterine Endometrioid Carcinoma |
| | | Rectal Adenocarcinoma |
| | | Mucinous Carcinoma |
| | | Head and Neck Squamous |
| | | Cell Carcinoma |
| | | Bladder Urothelial Carcinoma |
| 420 | C420R | Uterine Endometrioid Carcinoma |
| | | Tubular Stomach Adenocarcinoma |
| | | Lung Squamous Cell Carcinoma |
| | | Breast Invasive Ductal Carcinoma |
| | | Uterine Serous Carcinoma/Uterine |
| | | Papillary Serous |
| | | Carcinoma |
| | | Colon Adenocarcinoma |
| | | Intestinal Type Stomach |
| | | Adenocarcinoma |
| | | Stomach Adenocarcinoma |
| | | Head and Neck Squamous Cell |
| | | Carcinoma |
| | | Breast Invasive Carcinoma (NOS) |
| | | Astrocytoma |
| | | Cervical Squamous Cell Carcinoma |
| 432 | Y432C | Cervical Squamous Cell Carcinoma |
| 447 | P447_L455del (In | Breast Invasive Ductal Carcinoma |
| | frame Deletion) | |
| 449 | P449L, P449S | Uterine Endometrioid Carcinoma |
| 450 | H450_P458del | Breast Invasive Ductal Carcinoma |
| | (In Frame | |
| | Deletion) | |
| 451 | G451R, G451V, | Head and Neck Squamous |
| | G451_D454del | Cell Carcinoma |
| | (In Frame | Bladder Urothelial Carcinoma |
| | Deletion) | Colon Adenocarcinoma |
| 452 | L452_G460del | Breast Invasive Ductal Carcinoma |
| | (In Frame | |
| | Deletion) | |
| 453 | E453del, E453K, | Oligodendroglioma |
| | E453Q, | Uterine Mixed Endometrial |
| | E453_G460delins | Carcinoma |
| | DDF (in Frame | Intestinal Type Stomach |
| | Deletion), | Adenocarcinoma |
| | E453_L455del | Breast Invasive Ductal Carcinoma |
| | | Breast Invasive Lobular Carcinoma |
| | | Head and Neck Squamous Cell |
| | | Carcinoma |
| | | Astrocytoma |
| | | Stomach Adenocarcinoma |
| | | Bladder Urothelial Carcinoma |
| | | Lung Squamous Cell Carcinoma |
| | | Cervical Squamous Cell Carcinoma |
| | | Lung Adenocarcinoma |
| | | Mucinous Carcinoma |
| | | Uterine Endometrioid Carcinoma |
| | | Uterine Serous Carcinoma/Uterine |
| | | Papillary Serous |
| | | Carcinoma |
| | | Glioblastoma Multiforme |
| | | Colon Adenocarcinoma |
| 454 | D454Y | Uterine Endometrioid Carcinoma |
| 455 | L455_G463del | Glioblastoma Multiforme |
| | (In Frame | |
| | Deletion) | |
| 463 | G463_N465delins | Uterine Endometrioid Carcinoma |
| | D (In Frame | |
| | Deletion) | |
| 469 | E469A, | Rectal Adenocarcinoma |
| | E469delinsDK | Breast Invasive Ductal Carcinoma |
| | (In Frame | |
| | Insertion) | |
| 471 | P471A, P471L | Bladder Urothelial Carcinoma |
| | | Rectal Adenocarcinoma |
| | | Uterine Endometrioid Carcinoma |
| | | Oligoastrocytoma |
| | | Hepatocellular Carcinoma |
| 474 | E474A | Prostate Adenocarcinoma |
| 475 | L475F | Cutaneous Melanoma |
| 479 | W479* | Uterine Endometrioid Carcinoma |
| 495 | H495L, H495Y | Lung Squamous Cell Carcinoma |
| | | Uterine Endometrioid Carcinoma |
| 499 | S499F | Bladder Urothelial Carcinoma |
| 519 | R519G | Head and Neck Squamous Cell |
| | | Carcinoma |
| 520 | D520V | Breast Invasive Lobular Carcinoma |
| 522 | E522A | Uterine Endometrioid Carcinoma |
| 531 | L531V | Breast Invasive Ductal Carcinoma |
| 539 | P539R, P539S | Breast Invasive Ductal Carcinoma |
| | | Pancreatic Adenocarcinoma |
| | | Uterine Endometrioid Carcinoma |
| | | Lung Squamous Cell Carcinoma |
| 542 | E542A, E542G, | Uterine Serous Carcinoma/Uterine |
| | E542K, E542Q, | Papillary Serous |
| | E542V | Carcinoma |
| | | Uterine Endometrioid Carcinoma |
| | | Colon Adenocarcinoma |
| | | Prostate Adenocarcinoma |
| | | Breast Invasive Ductal Carcinoma |
| | | Breast Invasive Lobular Carcinoma |
| | | Endocervical Adenocarcinoma |
| | | Intestinal Type Stomach |
| | | Adenocarcinoma |
| | | Prostate Adenocarcinoma |
| | | Papillary Renal Cell Carcinoma |
| | | Oligoastrocytoma |
| | | Hepatocellular Carcinoma |
| | | Bladder Urothelial Carcinoma |
| | | Mucinous Adenocarcinoma of |
| | | the Colon and Rectum |
| | | Diffuse Type Stomach |
| | | Adenocarcinoma |
| | | Lung Squamous Cell Carcinoma |
| | | Signet Ring Cell Carcinoma |
| | | of the Stomach |
| | | Head and Neck Squamous Cell |
| | | Carcinoma |
| | | Breast Invasive Carcinoma (NOS) |
| | | Mucinous Carcinoma |
| | | Breast Invasive Ductal Carcinoma |
| | | Cervical Squamous Cell Carcinoma |
| | | Glioblastoma Multiforme |
| | | Lung Adenocarcinoma |
| 545 | E545A, E545D, | Uterine Serous Carcinoma/Uterine |
| | E545G, E545K, | Papillary Serous |
| | E545Q | Carcinoma |
| | | Uterine Endometrioid Carcinoma |
| | | Colon Adenocarcinoma |
| | | Prostate Adenocarcinoma |
| | | Breast Invasive Ductal Carcinoma |
| | | Breast Invasive Lobular Carcinoma |
| | | Endocervical Adenocarcinoma |
| | | Intestinal Type Stomach |
| | | Adenocarcinoma |
| | | Papillary Renal Cell Carcinoma |
| | | Oligoastrocytoma |
| | | Hepatocellular Carcinoma |
| | | Bladder Urothelial Carcinoma |
| | | Mucinous Adenocarcinoma of |
| | | the Colon and Rectum |
| | | Diffuse Type Stomach |
| | | Adenocarcinoma |
| | | Lung Squamous Cell Carcinoma |
| | | Signet Ring Cell Carcinoma |
| | | of the Stomach |
| | | Head and Neck Squamous Cell |
| | | Carcinoma |
| | | Breast Invasive Carcinoma (NOS) |
| | | Mucinous Carcinoma |
| | | Cervical Squamous Cell Carcinoma |
| | | Glioblastoma Multiforme |
| | | Oligodendroglioma |
| | | Lung Adenocarcinoma |
| | | Serous Ovarian Cancer |
| | | Uterine Carcinosarcoma/Uterine |
| | | Malignant Mixed |
| | | Mullerian Tumor |
| | | Astrocytoma |
| | | Rectal Adenocarcinoma |
| | | Stomach Adenocarcinoma |
| | | Cutaneous Melanoma |
| | | Esophageal Squamous Cell |
| | | Carcinoma |
| | | Breast Invasive Mixed Mucinous |
| | | Carcinoma |
| | | Intrahepatic Cholangiocarcinoma |
| | | Renal Clear Cell Carcinoma |
| | | Seminoma |
| | | Esophageal Adenocarcinoma |
| | | Tubular Stomach Adenocarcinoma |
| | | Uterine Mixed Endometrial |
| | | Carcinoma |
| 546 | Q546E, Q546H, | Uterine Endometrioid Carcinoma |
| | Q546K, Q546P, | Rectal Adenocarcinoma |
| | Q546R | Oligodendroglioma |
| | | Stomach Adenocarcinoma |
| | | Esophageal Adenocarcinoma |
| | | Bladder Urothelial Carcinoma |
| | | Breast Invasive Carcinoma (NOS) |
| | | Breast Invasive Ductal Carcinoma |
| | | Colon Adenocarcinoma |
| | | Glioblastoma Multiforme |
| | | Uterine Serous Carcinoma/Uterine |
| | | Papillary Serous |
| | | Carcinoma |
| | | Undifferentiated Pleomorphic |
| | | Sarcoma/Malignant Fibrous |
| | | Histiocytoma/High-Grade Spindle |
| | | Cell Sarcoma |
| | | Astrocytoma |
| | | Uterine Carcinosarcoma/Uterine |
| | | Malignant Mixed |
| | | Mullerian Tumor |
| | | Oligoastrocytoma |
| | | Breast Invasive Lobular Carcinoma |
| | | Tubular Stomach Adenocarcinoma |
| | | Head and Neck Squamous Cell |
| | | Carcinoma |
| | | Cervical Squamous Cell Carcinoma |
| | | Intestinal Type Stomach |
| | | Adenocarcinoma |
| 547 | E547D, E547K | Lung Squamous Cell Carcinoma |
| | | Stomach Adenocarcinoma |
| 552 | W552C | Bladder Urothelial Carcinoma |
| 569 | L569I | Uterine Serous Carcinoma/Uterine |
| | | Papillary Serous |
| | | Carcinoma |
| | | Prostate Adenocarcinoma |
| 576 | S576Y | Uterine Endometrioid Carcinoma |
| 581 | A581S | Cutaneous Melanoma |
| 589 | D589N | Cervical Squamous Cell Carcinoma |
| 600 | E600K, E600V | Uterine Endometrioid Carcinoma |
| | | Bladder Urothelial Carcinoma |
| | | Lung Adenocarcinoma |
| | | Breast Invasive Lobular Carcinoma |
| | | Papillary Stomach Adenocarcinoma |
| 603 | D603H | Breast Invasive Ductal Carcinoma |
| 604 | C604R | Uterine Endometrioid Carcinoma |
| | | Uterine Carcinosarcoma/Uterine |
| | | Malignant Mixed |
| | | Mullerian Tumor |
| | | Uterine Serous Carcinoma/Uterine |
| | | Papillary Serous |
| | | Carcinoma |
| | | Head and Neck Squamous Cell |
| | | Carcinoma |
| 606 | Y606C | Head and Neck Squamous Cell |
| | | Carcinoma |
| 607 | P607Q | Cutaneous Melanoma |
| 609 | P609H | Colon Adenocarcinoma |
| 614 | F614I | Breast Invasive Ductal Carcinoma |
| 617 | R617Q, R617W | Uterine Endometrioid Carcinoma |
| 617 | R617W | Uterine Endometrioid Carcinoma |
| 629 | S629C | Breast Invasive Ductal Carcinoma |
| 636 | V636L | Bladder Urothelial Carcinoma |
| 642 | E642K | Uterine Serous Carcinoma/Uterine |
| | | Papillary Serous |
| | | Carcinoma |
| 643 | Q643H | Uterine Endometrioid Carcinoma |
| 658 | L658F | Colon Adenocarcinoma |
| 667 | F667L | Uterine Endometrioid Carcinoma |
| | | Lung Squamous Cell Carcinoma |
| | | Uterine Serous Carcinoma/Uterine |
| | | Papillary Serous |
| | | Carcinoma |
| 673 | S673T | Breast Invasive Ductal Carcinoma |
| 674 | E674* (Nonsense | Papillary Thyroid Cancer |
| | mutation), | Cutaneous Melanoma |
| | E674D, E674Q | Bladder Urothelial Carcinoma |
| 682 | Q682K, | Cutaneous Melanoma |
| | Q682Rfs*18 | Glioblastoma Multiforme |
| | (Frame Shift | |
| | Deletion) | |
| 683 | R683M | Cutaneous Melanoma |
| 684 | F684L | Uterine Endometrioid Carcinoma |
| 693 | R693H | Cervical Squamous Cell Carcinoma |
| 710 | E710Q | Bladder Urothelial Carcinoma |
| 711 | K711N | Astrocytoma |
| 722 | E722K | Colon Adenocarcinoma |
| 725 | D725G, D725N | Colon Adenocarcinoma |
| | | Uterine Endometrioid Carcinoma |
| 726 | E726K | Cervical Squamous Cell Carcinoma |
| | | Uterine Endometrioid Carcinoma |
| | | Breast Invasive Ductal Carcinoma |
| | | Hepatocellular Carcinoma |
| | | Lung Adenocarcinoma |
| | | Esophageal Squamous Cell |
| | | Carcinoma |
| | | Esophageal Adenocarcinoma |
| | | Rectal Adenocarcinoma |
| | | Head and Neck Squamous Cell |
| | | Carcinoma |
| | | Lung Squamous Cell Carcinoma |
| | | Breast Invasive Lobular Carcinoma |
| | | Bladder Urothelial Carcinoma |
| | | Colon Adenocarcinoma |
| 729 | K729N | Cutaneous Melanoma |
| 732 | M732I | Colon Adenocarcinoma |
| 737 | E737K | Cutaneous Melanoma |
| 741 | R741Q | Serous Ovarian Cancer |
| 744 | F744I | Stomach Adenocarcinoma |
| 746 | D746Y | Cutaneous Melanoma |
| 749 | Q749H | Cutaneous Melanoma |
| 752 | L752V | Bladder Urothelial Carcinoma |
| 766 | L766F | Breast Invasive Ductal Carcinoma |
| 770 | R770Q | Uterine Endometrioid Carcinoma |
| 773 | S773F | Cutaneous Melanoma |
| 777 | R777M, R777K | Cutaneous Melanoma |
| | | Colon Adenocarcinoma |
| 791 | E791Q | Bladder Urothelial Carcinoma |
| 811 | M811I | Uterine Endometrioid Carcinoma |
| 816 | I816S | Uterine Endometrioid Carcinoma |
| 818 | R818C, R818H | Cutaneous Melanoma |
| | | Uterine Endometrioid Carcinoma |
| 849 | E849K | Serous Ovarian Cancer |
| 852 | R852Q | Leiomyosarcoma |
| | | Uterine Serous Carcinoma/Uterine |
| | | Papillary Serous |
| | | Carcinoma |
| 865 | G865D | Cutaneous Melanoma |
| 866 | L866F, L866W | Cervical Squamous Cell Carcinoma |
| | | Uterine Endometrioid Carcinoma |
| 879 | Q879R | Stomach Adenocarcinoma |
| 886 | K886E | Undifferentiated Pleomorphic |
| | | Sarcoma/Malignant Fibrous |
| | | Histiocytoma/High-Grade Spindle |
| | | Cell Sarcoma |
| 901 | C901F | Uterine Endometrioid Carcinoma |
| | | Astrocytoma |
| | | Breast Invasive Ductal Carcinoma |
| | | Head and Neck Squamous Cell |
| | | Carcinoma |
| 903 | G903E | Uterine Serous Carcinoma/Uterine |
| | | Papillary Serous |
| | | Carcinoma |
| 905 | C905S | Head and Neck Squamous Cell |
| | | Carcinoma |
| 909 | F909C | Esophageal Adenocarcinoma |
| 914 | G914R | Uterine Serous Carcinoma/Uterine |
| | | Papillary Serous |
| | | Carcinoma |
| | | Astrocytoma |
| 929 | L929M | Uterine Endometrioid Carcinoma |
| 930 | F930V | Uterine Endometrioid Carcinoma |
| 939 | D939G | Breast Invasive Carcinoma (NOS) |
| | | Uterine Endometrioid Carcinoma |
| | | Breast Invasive Ductal Carcinoma |
| 948 | K948E | Intestinal Type Stomach |
| | | Adenocarcinoma |
| 951 | R951C | Rectal Adenocarcinoma |
| 953 | P953S | Uterine Endometrioid Carcinoma |
| 956 | L956F | Bladder Urothelial Carcinoma |
| 958 | Q958R | Uterine Mixed Endometrial |
| | | Carcinoma |
| 970 | E970K | Esophageal Squamous Cell |
| | | Carcinoma |
| | | Head and Neck Squamous Cell |
| | | Carcinoma |
| | | Mucinous Adenocarcinoma of |
| | | the Colon and Rectum |
| | | Colon Adenocarcinoma |
| 971 | C971R | Head and Neck Squamous Cell |
| | | Carcinoma |
| 978 | E978K | Bladder Urothelial Carcinoma |
| 979 | R979G | Pancreatic Adenocarcinoma |
| 985 | Y985* | Pleural Mesothelioma, Biphasic Type |
| 989 | L989V | Breast Invasive Ductal Carcinoma |
| 992 | R992L, R992P | Bladder Urothelial Carcinoma |
| | | Uterine Serous Carcinoma/Uterine |
| | | Papillary Serous |
| | | Carcinoma |
| | | Mucinous Carcinoma |
| 997 | L997I | Uterine Endometrioid Carcinoma |
| 1002 | F1002L | Uterine Endometrioid Carcinoma |
| 1004 | M1004I, | Uterine Endometrioid Carcinoma |
| | M1004R, | Breast Invasive Ductal Carcinoma |
| | M1004V | Bladder Urothelial Carcinoma |
| | | Lung Squamous Cell Carcinoma |
| 1005 | M1005V | Oligodendroglioma |
| 1006 | L1006R | Uterine Endometrioid Carcinoma |
| 1007 | G1007R | Uterine Endometrioid Carcinoma |
| | | Breast Invasive Ductal Carcinoma |
| | | Head and Neck Squamous |
| | | Cell Carcinoma |
| | | Colon Adenocarcinoma |
| | | Endocervical Adenocarcinoma |
| 1012 | E1012Q | Bladder Urothelial Carcinoma |
| 1015 | S1015Y | Mucinous Adenocarcinoma of |
| | | the Colon and Rectum |
| 1016 | F1016C | Uterine Endometrioid Carcinoma |
| 1017 | D1017N | Pancreatic Adenocarcinoma |
| 1020 | A1020T | Uterine Endometrioid Carcinoma |
| 1021 | Y1021C, Y1021H | Uterine Carcinosarcoma/Uterine |
| | | Malignant Mixed |
| | | Mullerian Tumor |
| | | Colon Adenocarcinoma |
| | | Breast Invasive Ductal Carcinoma |
| | | Uterine Serous Carcinoma/Uterine |
| | | Papillary Serous |
| | | Carcinoma |
| | | Uterine Endometrioid Carcinoma |
| | | Stomach Adenocarcinoma |
| | | Mucinous Adenocarcinoma of |
| | | the Colon and Rectum |
| | | Tubular Stomach Adenocarcinoma |
| 1025 | T1025A, T1025S | Uterine Endometrioid Carcinoma |
| | | Breast Invasive Ductal Carcinoma |
| | | Mucinous Adenocarcinoma of |
| | | the Colon and Rectum |
| | | Uterine Mixed Endometrial |
| | | Carcinoma |
| 1023 | R1023Q^{†} | Colorectal Cancer |
| 1026 | L1026I | Cutaneous Melanoma |
| 1029 | D1029H | Uterine Serous Carcinoma/Uterine |
| | | Papillary Serous |
| | | Carcinoma |
| 1037 | E1037K | Breast Invasive Ductal Carcinoma |
| 1040 | M1040I, M1040V | Head and Neck Squamous |
| | | Cell Carcinoma |
| | | Breast Invasive Ductal Carcinoma |
| 1043 | M1043I, | Breast Invasive Lobular Carcinoma |
| | M1043L, | Tubular Stomach Adenocarcinoma |
| | M1043T, | Uterine Endometrioid Carcinoma |
| | M1043V | Mucinous Adenocarcinoma of |
| | | the Colon and Rectum |
| | | Papillary Thyroid Cancer |
| | | Esophageal Squamous Cell |
| | | Carcinoma |
| | | Colon Adenocarcinoma |
| | | Breast Invasive Ductal Carcinoma |
| | | Bladder Urothelial Carcinoma |
| | | Pancreatic Adenocarcinoma |
| | | Oligodendroglioma |
| | | Uterine Serous Carcinoma/Uterine |
| | | Papillary Serous |
| | | Carcinoma |
| | | Glioblastoma Multiforme |
| | | Head and Neck Squamous |
| | | Cell Carcinoma |
| 1044 | N1044I, N1044K, | Uterine Endometrioid Carcinoma |
| | N1044Y | Breast Invasive Ductal Carcinoma |
| 1045 | D1045A, | Uterine Endometrioid Carcinoma |
| | D1045V | Lung Squamous Cell Carcinoma |
| 1047 | H1047L, | Esophageal Squamous Cell |
| | H1047Q, | Carcinoma |
| | H1047R, H1047Y | Uterine Endometrioid Carcinoma |
| | | Hepatocellular Carcinoma |
| | | Cutaneous Melanoma |
| | | Mucinous Adenocarcinoma of |
| | | the Colon and Rectum |
| | | Bladder Urothelial Carcinoma |
| | | Cervical Squamous Cell Carcinoma |
| | | Intrahepatic Cholangiocarcinoma |
| | | Uterine Mixed Endometrial |
| | | Carcinoma |
| | | Breast Invasive Ductal Carcinoma |
| | | Renal Clear Cell Carcinoma |
| | | Uterine Serous Carcinoma/Uterine |
| | | Papillary Serous |
| | | Carcinoma |
| | | Head and Neck Squamous Cell |
| | | Carcinoma |
| | | Lung Squamous Cell Carcinoma |
| | | Breast Invasive Lobular |
| | | Carcinoma |
| | | Breast Invasive Carcinoma (NOS) |
| | | Astrocytoma |
| | | Colon Adenocarcinoma |
| | | Leiomyosarcoma |
| | | Uterine Carcinosarcoma/Uterine |
| | | Malignant Mixed |
| | | Mullerian Tumor |
| | | Oligodendroglioma |
| | | Serous Ovarian Cancer |
| | | Mucinous Stomach Adenocarcinoma |
| | | Rectal Adenocarcinoma |
| | | Intestinal Type Stomach |
| | | Adenocarcinoma |
| | | Diffuse Type Stomach |
| | | Adenocarcinoma |
| | | Prostate Adenocarcinoma |
| | | Lung Adenocarcinoma |
| | | Stomach Adenocarcinoma |
| | | Tubular Stomach Adenocarcinoma |
| | | Adrenocortical Carcinoma |
| | | Undifferentiated Pleomorphic |
| | | Sarcoma/Malignant Fibrous |
| | | Histiocytoma/High-Grade Spindle |
| | | Cell Sarcoma |
| | | Glioblastoma Multiforme |
| | | Oligoastrocytoma |
| 1048 | H1048R | Colon Adenocarcinoma |
| | | Renal Clear Cell Carcinoma |
| 1049 | G1049R | Intestinal Type Stomach |
| | | Adenocarcinoma |
| | | Bladder Urothelial Carcinoma |
| | | Renal Clear Cell Carcinoma |
| | | Breast Invasive Ductal Carcinoma |
| | | Breast Invasive Lobular Carcinoma |
| | | Uterine Endometrioid Carcinoma |
| | | Colon Adenocarcinoma |
| 1052 | T1052K | Hepatocellular Carcinoma |
| | | Colon Adenocarcinoma |
| 1055 | M1055I | Uterine Mixed Endometrial |
| | | Carcinoma |
| 1058 | I1058M | Uterine Carcinosarcoma/Uterine |
| | | Malignant Mixed |
| | | Mullerian Tumor |
| 1065 | H1065L | Breast Invasive Lobular |
| | | Carcinoma |
| 1066 | A1066V | Uterine Mixed Endometrial |
| | | Carcinoma |
| 1068 | N1068Y, | Pleural Mesothelioma, |
| | N1068fs*5 | Epithelioid Type |
| | (Frame Shift | Dedifferentiated Liposarcoma |
| | Insertion) | Head and Neck Squamous |
| | | Cell Carcinoma |
| 1069 | *1069Wext*4 | Glioblastoma Multiforme |
| | (nonstop | |
| | Mutation) | |

^{A}Unless noted otherwise, the mutations of Table 1 are found in cBioPortal database derived from Cerami et al. The cBio Cancer Genomics Portal: An Open Platform for Exploring Multidimensional Cancer Genomics Data. Cancer Discovery. May 2012 2; 401; and Gao et al. Integrative analysis of complex cancer genomics and clinical profiles using the cBioPortal. Sci. Signal. 6, pl1 (2013).
^{†}Velho S, Oliveira C, Ferreira A, Ferreira AC, Suriano G, Schwartz S Jr, Duval A, Carneiro F, Machado JC, Hamelin R, Seruca R. The prevalence of PIK3CA mutations in gastric and colon cancer. Eur J Cancer. 2005 Jul;41(11):1649-54. doi: 10.1016/j.ejca.2005.04.022. PMID: 15994075.

TABLE 2
Additional PI3Kα Protein Amino
Acid Substitutions/Insertions/Deletions^{A}

| Amino | Non-Limiting | |
|---|---|---|
| Acid | Exemplary | Non-Limiting Exemplary |
| Position | Mutations | PI3Kα Associated Cancer(s) |
| 1043 | M1043I, | Breast Invasive Lobular Carcinoma |
| | M1043L, | Tubular Stomach Adenocarcinoma |
| | M1043T, | Uterine Endometrioid Carcinoma |
| | M1043V | Mucinous Adenocarcinoma of the |
| | | Colon and Rectum |
| | | Papillary Thyroid Cancer |
| | | Esophageal Squamous Cell Carcinoma |
| | | Colon Adenocarcinoma |
| | | Breast Invasive Ductal Carcinoma |
| | | Bladder Urothelial Carcinoma |
| | | Pancreatic Adenocarcinoma |
| | | Oligodendroglioma |
| | | Uterine Serous Carcinoma/Uterine |
| | | Papillary Serous |
| | | Carcinoma |
| | | Glioblastoma Multiforme |
| | | Head and Neck Squamous Cell Carcinoma |
| 1044 | N1044I, N1044K, | Uterine Endometrioid Carcinoma |
| | N1044Y | Breast Invasive Ductal Carcinoma |
| 1045 | D1045A, | Uterine Endometrioid Carcinoma |
| | D1045V | Lung Squamous Cell Carcinoma |
| 1047 | H1047L, | Esophageal Squamous Cell Carcinoma |
| | H1047Q, | Uterine Endometrioid Carcinoma |
| | H1047R, H1047Y | Hepatocellular Carcinoma |
| | | Cutaneous Melanoma |
| | | Mucinous Adenocarcinoma of the |
| | | Colon and Rectum |
| | | Bladder Urothelial Carcinoma |
| | | Cervical Squamous Cell Carcinoma |
| | | Intrahepatic Cholangiocarcinoma |
| | | Uterine Mixed Endometrial Carcinoma |
| | | Breast Invasive Ductal Carcinoma |
| | | Renal Clear Cell Carcinoma |
| | | Uterine Serous Carcinoma/Uterine |
| | | Papillary Serous |
| | | Carcinoma |
| | | Head and Neck Squamous Cell Carcinoma |
| | | Lung Squamous Cell Carcinoma |
| | | Breast Invasive Lobular Carcinoma |
| | | Breast Invasive Carcinoma (NOS) |
| | | Astrocytoma |
| | | Colon Adenocarcinoma |
| | | Leiomyosarcoma |
| | | Uterine Carcinosarcoma/Uterine |
| | | Malignant Mixed |
| | | Mullerian Tumor |
| | | Oligodendroglioma |
| | | Serous Ovarian Cancer |
| | | Mucinous Stomach Adenocarcinoma |
| | | Rectal Adenocarcinoma |
| | | Intestinal Type Stomach Adenocarcinoma |
| | | Diffuse Type Stomach Adenocarcinoma |
| | | Prostate Adenocarcinoma |
| | | Lung Adenocarcinoma |
| | | Stomach Adenocarcinoma |
| | | Tubular Stomach Adenocarcinoma |
| | | Adrenocortical Carcinoma |
| | | Undifferentiated Pleomorphic |
| | | Sarcoma/Malignant Fibrous |
| | | Histiocytoma/High-Grade Spindle |
| | | Cell Sarcoma |
| | | Glioblastoma Multiforme |
| | | Oligoastrocytoma |
| 1048 | H1048R | Colon Adenocarcinoma |
| | | Renal Clear Cell Carcinoma |
| 1049 | G1049R | Intestinal Type Stomach Adenocarcinoma |
| | | Bladder Urothelial Carcinoma |
| | | Renal Clear Cell Carcinoma |
| | | Breast Invasive Ductal Carcinoma |
| | | Breast Invasive Lobular Carcinoma |
| | | Uterine Endometrioid Carcinoma |
| | | Colon Adenocarcinoma |
| 1052 | T1052K | Hepatocellular Carcinoma |
| | | Colon Adenocarcinoma |
| 1055 | M1055I | Uterine Mixed Endometrial Carcinoma |
| 1058 | I1058M | Uterine Carcinosarcoma/Uterine |
| | | Malignant Mixed |
| | | Mullerian Tumor |
| 1065 | H1065L | Breast Invasive Lobular Carcinoma |
| 1066 | A1066V | Uterine Mixed Endometrial Carcinoma |
| 1068 | N1068Y, | Pleural Mesothelioma, Epithelioid Type |
| | N1068fs*5 | Dedifferentiated Liposarcoma |
| | (Frame Shift | Head and Neck Squamous Cell Carcinoma |
| | Insertion) | |

^{A}Unless noted otherwise, the mutations of Table 2 are found in cBioPortal database derived from Cerami et al. The cBio Cancer Genomics Portal: An Open Platform for Exploring Multidimensional Cancer Genomics Data. Cancer Discovery. May 2012 2; 401; and Gao et al. Integrative analysis of complex cancer genomics and clinical profiles using the cBioPortal. Sci. Signal. 6, pl1 (2013).
^{†} Velho S, Oliveira C, Ferreira A, Ferreira AC, Suriano G, Schwartz S Jr, Duval A, Carneiro F, Machado JC, Hamelin R, Seruca R. The prevalence of PIK3CA mutations in gastric and colon cancer. Eur J Cancer. 2005 Jul;41(11):1649-54. doi: 10.1016/j.ejca.2005.04.022. PMID: 15994075.

In some embodiments, the dysregulation of a PIK3CA gene, a PI3Kα protein, or expression or activity or level of any of the same, includes a splice variation in a PI3Kα mRNA which results in an expressed protein that is an alternatively spliced variant of PI3Kα having at least one residue deleted (as compared to the wild type PI3Kα protein) resulting in a constitutive activity of a PI3Kα protein domain.

In some embodiments, the dysregulation of a PIK3CA gene, a PI3Kα protein, or expression or activity or level of any of the same, includes at least one point mutation in a PIK3CA gene that results in the production of a PI3Kα protein that has one or more amino acid substitutions or insertions or deletions in a PIK3CA gene that results in the production of a PI3Kα protein that has one or more amino acids inserted or removed, as compared to the wild type PI3Kα protein. In some cases, the resulting mutant PI3Kα protein has increased activity, as compared to a wild type PI3Kα protein or a PI3Kα protein not including the same mutation. In some embodiments, the compounds described herein selectively inhibit the resulting mutant PI3Kα protein relative to a wild type PI3Kα protein or a PI3Kα protein not including the same mutation.

Exemplary Sequence of Human Phosphatidylinositol 4,5-bisphosphate 3-kinase isoform alpha (UniProtKB entry P42336) (SEQ ID NO: 1)

MPPRPSSGEL WGIHLMPPRIL VECLLPNGM IVTLECLREA
TLITIKHELF KEARKYPLHQ LLQDESSYIF VSVTQEAERE
EFFDETRRLC DLRLFQPFLK VIEPVGNREE KILNREIGFA
IGMPVCEFDM VKDPEVQDFR RNILNVCKEA VDLRDLNSPH
SRAMYVYPPN VESSPELPKH IYNKLDKGQI IVVIWVIVSP
NNDKQKYTLK INHDCVPEQV IAEAIRKKTR SMLLSSEQLK
LCVLEYQGKY ILKVCGCDEY FLEKYPLSQY KYIRSCIMLG
RMPNLMLMAK ESLYSQLPMD CFTMPSYSRR ISTATPYMNG
ETSTKSLWVI NSALRIKILC ATYVNVNIRD IDKIYVRTGI
YHGGEPLCDN VNTQRVPCSN PRWNEWLNYD IYIPDLPRAA
RLCLSICSVK GRKGAKEEHC PLAWGNINLF DYTDTLVSGK
MALNLWPVPH GLEDLLNPIG VTGSNPNKET PCLELEFDWF
SSVVKFPDMS VIEEHANWSV SREAGFSYSH AGLSNRLARD
NELRENDKEQ LKAISTRDPL SEITEQEKDF LWSHRHYCVT
IPEILPKLLL SVKWNSRDEV AQMYCLVKDW PPIKPEQAME
LLDCNYPDPM VRGFAVRCLE KYLTDDKLSQ YLIQLVQVLK
YEQYLDNLLV RFLLKKALTN QRIGHFFFWH LKSEMHNKTV
SQRFGLLLES YCRACGMYLK HLNRQVEAME KLINLTDILK
QEKKDETQKV QMKFLVEQMR RPDFMDALQG FLSPLNPAHQ
LGNLRLEECR IMSSAKRPLW LNWENPDIMS ELLFQNNEII
FKNGDDLRQD MLTLQIIRIM ENIWQNQGLD LRMLPYGCLS
IGDCVGLIEV VRNSHTIMQI QCKGGLKGAL QFNSHTLHQW
LKDKNKGEIY DAAIDLFTRS CAGYCVATFI LGIGDRHNSN
IMVKDDGQLF HIDFGHFLDH KKKKFGYKRE RVPFVLTQDF
LIVISKGAQE CTKTREFERF QEMCYKAYLA IRQHANLFIN
LFSMMLGSGM PELQSFDDIA YIRKTLALDK TEQEALEYFM
KQMNDAHHGG WTTKMDWIFH TIKQHALN

In some embodiments, compounds of Formula (I), or pharmaceutically acceptable thereof, are useful for treating a cancer that has been identified as having one or more PI3Kα mutations. Accordingly, provided herein are methods for treating a subject diagnosed with (or identified as having) a cancer that include administering to the subject a therapeutically effective amount of a compound of Formula (I), or a pharmaceutically acceptable salt thereof.

Also provided herein are methods for treating a subject identified or diagnosed as having a PI3Kα-associated cancer that include administering to the subject a therapeutically effective amount of a compound of Formula (I), or a pharmaceutically acceptable salt thereof, or a pharmaceutical composition thereof. In some embodiments, the subject that has been identified or diagnosed as having a PI3Kα-associated cancer through the use of a regulatory agency-approved, e.g., FDA-approved test or assay for identifying dysregulation of a PIK3CA gene, a PI3Kα protein, or expression or activity or level of any of the same, in a subject or a biopsy sample from the subject or by performing any of the non-limiting examples of assays described herein. In some embodiments, the test or assay is provided as a kit. In some embodiments, the cancer is an PI3Kα-associated cancer.

The term “regulatory agency” refers to a country's agency for the approval of the medical use of pharmaceutical agents with the country. For example, a non-limiting example of a regulatory agency is the U.S. Food and Drug Administration (FDA).

Also provided are methods for treating cancer in a subject in need thereof, the method comprising: (a) detecting a PI3Kα-associated cancer in the subject; and (b) administering to the subject a therapeutically effective amount of a compound of Formula (I), or a pharmaceutically acceptable salt thereof, or a pharmaceutical composition thereof. Some embodiments of these methods further include administering to the subject another anticancer agent (e.g., an immunotherapy). In some embodiments, the subject was previously treated with another anticancer treatment, e.g., at least partial resection of the tumor or radiation therapy. In some embodiments, the subject is determined to have a PI3Kα-associated cancer through the use of a regulatory agency-approved, e.g., FDA-approved test or assay for identifying dysregulation of a PIK3CA gene, a PI3Kα protein, or expression or activity or level of any of the same, in a subject or a biopsy sample from the subject or by performing any of the non-limiting examples of assays described herein. In some embodiments, the test or assay is provided as a kit. In some embodiments, the cancer is an PI3Kα-associated cancer.

Also provided are methods of treating a subject that include performing an assay on a sample obtained from the subject to determine whether the subject has a dysregulation of a PIK3CA gene, a PI3Kα protein, or expression or activity or level of any of the same, and administering (e.g., specifically or selectively administering) a therapeutically effective amount of a compound of Formula (I), or a pharmaceutically acceptable salt thereof, or a pharmaceutical composition thereof, to the subject determined to have a dysregulation of a PIK3CA gene, a PI3Kα protein, or expression or activity or level of any of the same. Some embodiments of these methods further include administering to the subject another anticancer agent (e.g., an immunotherapy). In some embodiments of these methods, the subject was previously treated with another anticancer treatment, e.g., at least partial resection of a tumor or radiation therapy. In some embodiments, the subject is a subject suspected of having a PI3Kα-associated cancer, a subject presenting with one or more symptoms of a PI3Kα-associated cancer, or a subject having an elevated risk of developing a PI3Kα-associated cancer. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy. Additional, non-limiting assays that may be used in these methods are described herein. Additional assays are also known in the art.

Also provided is a compound of Formula (I), or a pharmaceutically acceptable salt thereof, or a pharmaceutical composition thereof, for use in treating a PI3Kα-associated cancer in a subject identified or diagnosed as having a PI3Kα-associated cancer through a step of performing an assay (e.g., an in vitro assay) on a sample obtained from the subject to determine whether the subject has a dysregulation of a PIK3CA gene, a PI3Kα protein, or expression or activity or level of any of the same, where the presence of a dysregulation of a PIK3CA gene, a PI3Kα protein, or expression or activity or level of any of the same, identifies that the subject has a PI3Kα-associated cancer. Also provided is the use of a compound of Formula (I), or a pharmaceutically acceptable salt thereof, for the manufacture of a medicament for treating a PI3Kα-associated cancer in a subject identified or diagnosed as having a PI3Kα-associated cancer through a step of performing an assay on a sample obtained from the subject to determine whether the subject has a dysregulation of a PIK3CA gene, a PI3Kα protein, or expression or activity or level of any of the same where the presence of dysregulation of a PIK3CA gene, a PI3Kα protein, or expression or activity or level of any of the same, identifies that the subject has a PI3Kα-associated cancer. Some embodiments of any of the methods or uses described herein further include recording in the subject's clinical record (e.g., a computer readable medium) that the subject is determined to have a dysregulation of a PIK3CA gene, a PI3Kα protein, or expression or activity or level of any of the same, through the performance of the assay, should be administered a compound of Formula (I), or a pharmaceutically acceptable salt thereof, or a pharmaceutical composition thereof. In some embodiments, the assay utilizes next generation sequencing, pyrosequencing, immunohistochemistry, or break apart FISH analysis. In some embodiments, the assay is a regulatory agency-approved assay, e.g., FDA-approved kit. In some embodiments, the assay is a liquid biopsy.

Also provided is a compound of Formula (I), or a pharmaceutically acceptable salt thereof, for use in the treatment of a cancer in a subject in need thereof, or a subject identified or diagnosed as having a PI3Kα-associated cancer. Also provided is the use of a compound of Formula (I), or a pharmaceutically acceptable salt thereof, for the manufacture of a medicament for treating a cancer in a subject identified or diagnosed as having a PI3Kα-associated cancer. In some embodiments, a subject is identified or diagnosed as having a PI3Kα-associated cancer through the use of a regulatory agency-approved, e.g., FDA-approved, kit for identifying dysregulation of a PIK3CA gene, a PI3Kα protein, or expression or activity or level of any of the same, in a subject or a biopsy sample from the subject. As provided herein, a PI3Kα-associated cancer includes those described herein and known in the art.

In some embodiments of any of the methods or uses described herein, the subject has been identified or diagnosed as having a cancer with a dysregulation of a PIK3CA gene, a PI3Kα protein, or expression or activity or level of any of the same. In some embodiments of any of the methods or uses described herein, the subject has a tumor that is positive for a dysregulation of a PIK3CA gene, a PI3Kα protein, or expression or activity or level of any of the same. In some embodiments of any of the methods or uses described herein, the subject can be a subject with a tumor(s) that is positive for a dysregulation of a PIK3CA gene, a PI3Kα protein, or expression or activity or level of any of the same. In some embodiments of any of the methods or uses described herein, the subject can be a subject whose tumors have a dysregulation of a PIK3CA gene, a PI3Kα protein, or expression or activity or level of any of the same. In some embodiments of any of the methods or uses described herein, the subject is suspected of having a PI3Kα-associated cancer. In some embodiments, provided herein are methods for treating a PI3Kα-associated cancer in a subject in need of such treatment, the method comprising a) detecting a dysregulation of a PIK3CA gene, a PI3Kα protein, or the expression or activity or level of any of the same in a sample from the subject; and b) administering a therapeutically effective amount of a compound of Formula (I), or a pharmaceutically acceptable salt thereof. In some embodiments, the dysregulation of a PIK3CA gene, a PI3Kα protein, or the expression or activity or level of any of the same includes one or more PI3Kα protein point mutations/insertions/deletions. Non-limiting examples of PI3Kα protein point mutations/insertions/deletions are described in Table 1 and Table 2. In some embodiments, the PI3Kα protein point mutation/insertion/deletion is H1047X, where X is any amino acid. In some embodiments, the PI3Kα protein point mutations/insertions/deletions are selected from the group consisting of E542A, E542G, E542K, E542Q, E542V, E545A, E545D, E545G, E545K, E545Q, M10431, M1043L, M1043T, M1043V, H1047L, H1047Q, H1047R, H1047Y, and G1049R. In some embodiments, the cancer with a dysregulation of a PIK3CA gene, a PI3Kα protein, or expression or activity or level of any of the same is determined using a regulatory agency-approved, e.g., FDA-approved, assay or kit. In some embodiments, the tumor with a dysregulation of a PIK3CA gene, a PI3Kα protein, or expression or activity or level of any of the same is determined using a regulatory agency-approved, e.g., FDA-approved, assay or kit.

In some embodiments of any of the methods or uses described herein, the subject has a clinical record indicating that the subject has a tumor that has a dysregulation of a PIK3CA gene, a PI3Kα protein, or expression or activity or level of any of the same. Also provided are methods of treating a subject that include administering a therapeutically effective amount of a compound of Formula (I), or a pharmaceutically acceptable salt thereof, to a subject having a clinical record that indicates that the subject has a dysregulation of a PIK3CA gene, a PI3Kα protein, or expression or activity or level of any of the same.

In some embodiments, the methods provided herein include performing an assay on a sample obtained from the subject to determine whether the subject has a dysregulation of a PIK3CA gene, a PI3Kα protein, or expression or level of any of the same. In some such embodiments, the method also includes administering to a subject determined to have a dysregulation of a PIK3CA gene, a PI3Kα protein, or expression or activity, or level of any of the same a therapeutically effective amount of a compound of Formula (I), or a pharmaceutically acceptable salt thereof. In some embodiments, the method includes determining that a subject has a dysregulation of a PIK3CA gene, a PI3Kα protein, or expression or level of any of the same via an assay performed on a sample obtained from the subject. In such embodiments, the method also includes administering to a subject a therapeutically effective amount of a compound of Formula (I), or a pharmaceutically acceptable salt thereof. In some embodiments, the dysregulation in a PIK3CA gene, a PI3Kα protein, or expression or activity or level of any of the same is one or more point mutation in the PIK3CA gene (e.g., any of the one or more of the PI3Kα point mutations described herein). The one or more point mutations in a PIK3CA gene can result, e.g., in the translation of a PI3Kα protein having one or more of the following amino acid substitutions, deletions, and insertions: E542A, E542G, E542K, E542Q, E542V, E545A, E545D, E545G, E545K, E545Q, M10431, M1043L, M1043T, M1043V, H1047L, H1047Q, H1047R, H1047Y, and G1049R. The one or more mutations in a PIK3CA gene can result, e.g., in the translation of an PI3Kα protein having one or more of the following amino acids: 542, 545, 1043, and 1047 and 1049. In some embodiments, the dysregulation in a PIK3CA gene, a PI3Kα protein protein, or expression or activity or level of any of the same is one or more PI3Kα amino acid substitutions (e.g., any of the PI3Kα amino acid substitution described herein). Some embodiments of these methods further include administering to the subject another anticancer agent (e.g., an immunotherapy).

In some embodiments of any of the methods or uses described herein, an assay used to determine whether the subject has a dysregulation of a PIK3CA gene, or a PI3Kα protein, or expression or activity or level of any of the same, using a sample from a subject can include, for example, next generation sequencing, immunohistochemistry, fluorescence microscopy, break apart FISH analysis, Southern blotting, Western blotting, FACS analysis, Northern blotting, and PCR-based amplification (e.g., RT-PCR and quantitative real-time RT-PCR). As is well-known in the art, the assays are typically performed, e.g., with at least one labeled nucleic acid probe or at least one labeled antibody or antigen-binding fragment thereof. Assays can utilize other detection methods known in the art for detecting dysregulation of a PIK3CA gene, a PI3Kα protein, or expression or activity or levels of any of the same (see, e.g., the references cited herein). In some embodiments, the sample is a biological sample or a biopsy sample (e.g., a paraffin-embedded biopsy sample) from the subject. In some embodiments, the subject is a subject suspected of having a PI3Kα-associated cancer, a subject having one or more symptoms of a PI3Kα-associated cancer, and/or a subject that has an increased risk of developing a PI3Kα-associated cancer).

In some embodiments, dysregulation of a PIK3CA gene, a PI3Kα protein, or the expression or activity or level of any of the same can be identified using a liquid biopsy (variously referred to as a fluid biopsy or fluid phase biopsy). See, e.g., Karachialiou et al., “Real-time liquid biopsies become a reality in cancer treatment”, Ann. Transl. Med., 3(3):36, 2016. Liquid biopsy methods can be used to detect total tumor burden and/or the dysregulation of a PIK3CA gene, a PI3Kα protein, or the expression or activity or level of any of the same. Liquid biopsies can be performed on biological samples obtained relatively easily from a subject (e.g., via a simple blood draw) and are generally less invasive than traditional methods used to detect tumor burden and/or dysregulation of a PIK3CA gene, a PI3Kα protein, or the expression or activity or level of any of the same. In some embodiments, liquid biopsies can be used to detect the presence of dysregulation of a PIK3CA gene, a PI3Kα protein, or the expression or activity or level of any of the same at an earlier stage than traditional methods. In some embodiments, the biological sample to be used in a liquid biopsy can include, blood, plasma, urine, cerebrospinal fluid, saliva, sputum, broncho-alveolar lavage, bile, lymphatic fluid, cyst fluid, stool, ascites, and combinations thereof. In some embodiments, a liquid biopsy can be used to detect circulating tumor cells (CTCs). In some embodiments, a liquid biopsy can be used to detect cell-free DNA. In some embodiments, cell-free DNA detected using a liquid biopsy is circulating tumor DNA (ctDNA) that is derived from tumor cells. Analysis of ctDNA (e.g., using sensitive detection techniques such as, without limitation, next-generation sequencing (NGS), traditional PCR, digital PCR, or microarray analysis) can be used to identify dysregulation of a PIK3CA gene, a PI3Kα protein, or the expression or activity or level of any of the same.

Also provided is a method for inhibiting PI3Kα activity in a cell, comprising contacting the cell with a compound of Formula (I), or a pharmaceutically acceptable salt thereof. In some embodiments, the contacting is in vitro. In some embodiments, the contacting is in vivo. In some embodiments, the contacting is in vivo, wherein the method comprises administering an effective amount of a compound of Formula (I), or a pharmaceutically acceptable salt thereof, to a subject having a cell having aberrant PI3Kα activity. In some embodiments, the cell is a cancer cell. In some embodiments, the cancer cell is any cancer as described herein. In some embodiments, the cancer cell is a PI3Kα-associated cancer cell. As used herein, the term “contacting” refers to the bringing together of indicated moieties in an in vitro system or an in vivo system. For example, “contacting” a PI3Kα protein with a compound provided herein includes the administration of a compound provided herein to an individual or subject, such as a human, having a PI3Kα protein, as well as, for example, introducing a compound provided herein into a sample containing a cellular or purified preparation containing the PI3Kα protein.

Also provided herein is a method of inhibiting cell proliferation, in vitro or in vivo, the method comprising contacting a cell with an effective amount of a compound of Formula (I), or a pharmaceutically acceptable salt thereof, or a pharmaceutical composition thereof as defined herein.

Further provided herein is a method of increase cell death, in vitro or in vivo, the method comprising contacting a cell with an effective amount of a compound of Formula (I), or a pharmaceutically acceptable salt thereof, or a pharmaceutical composition thereof as defined herein. Also provided herein is a method of increasing tumor cell death in a subject. The method comprises administering to the subject an effective compound of Formula (I), or a pharmaceutically acceptable salt thereof, in an amount effective to increase tumor cell death.

The phrase “therapeutically effective amount” means an amount of compound that, when administered to a subject in need of such treatment, is sufficient to (i) treat a PI3Kα protein-associated disease or disorder, (ii) attenuate, ameliorate, or eliminate one or more symptoms of the particular disease, condition, or disorder, or (iii) delay the onset of one or more symptoms of the particular disease, condition, or disorder described herein. The amount of a compound of Formula (I), or a pharmaceutically acceptable salt thereof, that will correspond to such an amount will vary depending upon factors such as the particular compound, disease condition and its severity, the identity (e.g., weight) of the subject in need of treatment, but can nevertheless be routinely determined by one skilled in the art.

When employed as pharmaceuticals, the compounds of Formula (I), including pharmaceutically acceptable salts thereof, can be administered in the form of pharmaceutical compositions as described herein.

### Combinations

In the field of medical oncology it is normal practice to use a combination of different forms of treatment to treat each subject with cancer. In medical oncology the other component(s) of such conjoint treatment or therapy in addition to compositions provided herein may be, for example, surgery, radiotherapy, and chemotherapeutic agents, such as other kinase inhibitors, signal transduction inhibitors and/or monoclonal antibodies. For example, a surgery may be open surgery or minimally invasive surgery. Compounds of Formula (I), or pharmaceutically acceptable salts thereof, therefore may also be useful as adjuvants to cancer treatment, that is, they can be used in combination with one or more additional therapies or therapeutic agents, for example, a chemotherapeutic agent that works by the same or by a different mechanism of action. In some embodiments, a compound of Formula (I), or a pharmaceutically acceptable salt thereof, can be used prior to administration of an additional therapeutic agent or additional therapy. For example, a subject in need thereof can be administered one or more doses of a compound of Formula (I), or a pharmaceutically acceptable salt thereof, for a period of time and then undergo at least partial resection of the tumor. In some embodiments, the treatment with one or more doses of a compound of Formula (I), or a pharmaceutically acceptable salt thereof, reduces the size of the tumor (e.g., the tumor burden) prior to the at least partial resection of the tumor. In some embodiments, a subject in need thereof can be administered one or more doses of a compound of Formula (I), or a pharmaceutically acceptable salt thereof, for a period of time and under one or more rounds of radiation therapy. In some embodiments, the treatment with one or more doses of a compound of Formula (I), or a pharmaceutically acceptable salt thereof, reduces the size of the tumor (e.g., the tumor burden) prior to the one or more rounds of radiation therapy.

In some embodiments, a subject has a cancer (e.g., a locally advanced or metastatic tumor) that is refractory or intolerant to standard therapy (e.g., administration of a chemotherapeutic agent, such as a multi-kinase inhibitor, immunotherapy, or radiation (e.g., radioactive iodine)). In some embodiments, a subject has a cancer (e.g., a locally advanced or metastatic tumor) that is refractory or intolerant to prior therapy (e.g., administration of a chemotherapeutic agent, such as a multi-kinase inhibitor, immunotherapy, or radiation (e.g., radioactive iodine)). In some embodiments, a subject has a cancer (e.g., a locally advanced or metastatic tumor) that has no standard therapy. In some embodiments, a subject is PI3Kα inhibitor naive. For example, the subject is naive to treatment with a selective PI3Kα inhibitor. In some embodiments, a subject is not PI3Kα inhibitor naive. In some embodiments, a subject is kinase inhibitor naive. In some embodiments, a subject is not kinase inhibitor naive. In some embodiments, a subject has undergone prior therapy. For example, treatment with a multi-kinase inhibitor (MKI) or another PI3K inhibitor, such as buparlisib (BKM120), alpelisib (BYL719), WX-037, copanlisib (ALIQOPA™, BAY80-6946), dactolisib (NVP-BEZ235, BEZ-235), taselisib (GDC-0032, RG7604), sonolisib (PX-866), CUDC-907, PQR309, ZSTK474, SF1126, AZD8835, GDC-0077, ASN003, pictilisib (GDC-0941), pilaralisib (XL147, SAR245408), gedatolisib (PF-05212384, PKI-587), serabelisib (TAK-117, MLN1117, INK 1117), BGT-226 (NVP-BGT226), PF-04691502, apitolisib (GDC-0980), omipalisib (GSK2126458, GSK458), voxtalisib (XL756, SAR245409), AMG 511, CH5132799, GSK1059615, GDC-0084 (R^{G7666}), VS-5584 (SB2343), PKI-402, wortmannin, LY294002, PI-103, rigosertib, XL-765, LY2023414, SAR260301, KIN-193 (AZD-6428), GS-9820, AMG319, or GSK2636771.

In some embodiments of any the methods described herein, the compound of Formula (I) (or a pharmaceutically acceptable salt thereof) is administered in combination with a therapeutically effective amount of at least one additional therapeutic agent selected from one or more additional therapies or therapeutic (e.g., chemotherapeutic) agents.

Non-limiting examples of additional therapeutic agents include: other PI3Kα-targeted therapeutic agents (i.e., other PI3Kα inhibitors), EGFR inhibitors, HER2 inhibitors, RAS pathway targeted therapeutic agents (including mTOR inhibitors, as described herein), PARP inhibitors, other kinase inhibitors (e.g., receptor tyrosine kinase-targeted therapeutic agents (e.g., Trk inhibitors or multi-kinase inhibitors)), farnesyl transferase inhibitors, signal transduction pathway inhibitors, aromatase inhibitors, selective estrogen receptor modulators or degraders (SERMs/SERDs), checkpoint inhibitors, modulators of the apoptosis pathway (e.g., obataclax); cytotoxic chemotherapeutics, angiogenesis-targeted therapies, immune-targeted agents, including immunotherapy, and radiotherapy.

In some embodiments, the EGFR inhibitor is osimertinib (AZD9291, merelectinib, TAGRISSO™), erlotinib (TARCEVA®), gefitinib (IRESSA@), cetuximab (ERBITUX®), necitumumab (PORTRAZZA™, IMC-11F8), neratinib (HKI-272, NERLYNX®), lapatinib (TYKERB®), panitumumab (ABX-EGF, VECTIBIX®), vandetanib (CAPRELSA®), rociletinib (CO-1686), olmutinib (OLITA™, HM61713, BI-1482694), naquotinib (ASP8273), nazartinib (EGF816, NVS-816), PF-06747775, icotinib (BPI-2009H), afatinib (BIBW 2992, GILOTRIF®), dacomitinib (PF-00299804, PF-804, PF-299, PF-299804), avitinib (ACO0010), ACO1OMA EAI045, matuzumab (EMD-7200), nimotuzumab (h-R3, BIOMAb EGFR®), zalutumab, MDX447, depatuxizumab (humanized mAb 806, ABT-806), depatuxizumab mafodotin (ABT-414), ABT-806, mAb 806, canertinib (CI-1033), shikonin, shikonin derivatives (e.g., deoxyshikonin, isobutyrylshikonin, acetylshikonin, 0,0-dimethylacrylshikonin and acetylalkannin), poziotinib (NOV120101, HM781-36B), AV-412, ibrutinib, WZ4002, brigatinib (AP26113, ALUNBRIG®), pelitinib (EKB-569), tarloxotinib (TH-4000, PR610), BPI-15086, Hemay022, ZN-e4, tesevatinib (KD019, XL647), YH25448, epitinib (HMPL-813), CK-101, MM-151, AZD3759, ZD6474, PF-06459988, varlintinib (ASLAN001, ARRY-334543), AP32788, HLX07, D-0316, AEE788, HS-10296, avitinib, GW572016, pyrotinib (SHR1258), SCT200, CPGJ602, Sym004, MAb-425, Modotuximab (TAB-H49), futuximab (992 DS), zalutumumab, KL-140, R05083945, IMGN289, JNJ-61186372, LY3164530, Sym013, AMG 595, BDTX-189, avatinib, Disruptin, CL-387785, EGFRBi-Armed Autologous T Cells, and EGFR CAR-T Therapy. In some embodiments, the EGFR-targeted therapeutic agent is selected from osimertinib, gefitinib, erlotinib, afatinib, lapatinib, neratinib, AZD-9291, CL-387785, CO-1686, or WZ4002.

Exemplary HER2 inhibitors include trastuzumab (e.g., TRAZIMERA™, HERCEPTIN®), pertuzumab (e.g., PERJETA®), trastuzumab emtansine (T-DM1 or ado-trastuzumab emtansine, e.g., KADCYLA®), lapatinib, KU004, neratinib (e.g., NERLYNX®), dacomitinib (e.g., VIZIMPRO®), afatinib (GILOTRIF®), tucatinib (e.g., TUKYSA™), erlotinib (e.g., TARCEVA®), pyrotinib, poziotinib, CP-724714, CUDC-101, sapitinib (AZD8931), tanespimycin (17-AAG), IPI-504, PF299, pelitinib, S-22261 1, and AEE-788.

A “RAS pathway targeted therapeutic agent” as used herein includes any compound exhibiting inactivation activity of any protein in a RAS pathway (e.g., kinase inhibition, allosteric inhibition, inhibition of dimerization, and induction of degradation). Non-limiting examples of a protein in a RAS pathway include any one of the proteins in the RAS-RAF-MAPK pathway or PI3K/AKT pathway such as RAS (e.g., KRAS, HRAS, and NRAS), RAF (ARAF, BRAF, CRAF), MEK, ERK, PI3K, AKT, and mTOR. In some embodiments, a RAS pathway modulator can be selective for a protein in a RAS pathway, e.g., the RAS pathway modulator can be selective for RAS (also referred to as a RAS modulator). In some embodiments, a RAS modulator is a covalent inhibitor. In some embodiments, a RAS pathway targeted therapeutic agent is a “KRAS pathway modulator.” A KRAS pathway modulator includes any compound exhibiting inactivation activity of any protein in a KRAS pathway (e.g., kinase inhibition, allosteric inhibition, inhibition of dimerization, and induction of degradation). Non-limiting examples of a protein in a KRAS pathway include any one of the proteins in the KRAS-RAF-MAPK pathway or PI3K/AKT pathway such as KRAS, RAF, BRAF, MEK, ERK, PI3K (i.e., other PI3K inhibitors, as described herein), AKT, and mTOR. In some embodiments, a KRAS pathway modulator can be selective for a protein in a RAS pathway, e.g., the KRAS pathway modulator can be selective for KRAS (also referred to as a KRAS modulator). In some embodiments, a KRAS modulator is a covalent inhibitor.

Non-limiting examples of a KRAS-targeted therapeutic agents (e.g., KRAS inhibitors) include BI 1701963, AMG 510, ARS-3248, ARS1620, AZD4785, SML-8-73-1, SML-10-70-1, VSA9, AA12, and MRTX-849.

Further non-limiting examples of RAS-targeted therapeutic agents include BRAF inhibitors, MEK inhibitors, ERK inhibitors, PI3K inhibitors, AKT inhibitors, and mTOR inhibitors. In some embodiments, the BRAF inhibitor is vemurafenib (ZELBORAF®), dabrafenib (TAFINLAR®), and encorafenib (BRAFTOVI®), BMS-908662 (XL281), sorafenib, PLX3603, RAF265, R05185426, GSK2118436, ARQ 736, GDC-0879, PLX-4720, AZ304, PLX-8394, HM95573, RO5126766, LXH254, or a combination thereof.

In some embodiments, the MEK inhibitor is trametinib (MEKINIST®, GSK1120212), cobimetinib (COTELLIC®), binimetinib (MEKTOVI®, MEK162), selumetinib (AZD6244), PD0325901, MSC1936369B, SHR7390, TAK-733, R05126766, CS3006, WX-554, PD98059, CI1040 (PD184352), hypothemycin, or a combination thereof.

In some embodiments, the ERK inhibitor is FRI-20 (ON-01060), VTX-Ile, 25-OH-D3-3-BE (B3CD, bromoacetoxycalcidiol), FR-180204, AEZ-131 (AEZS-131), AEZS-136, AZ-13767370, BL-EI-001, LY-3214996, LTT-462, KO-947, KO-947, MK-8353 (SCH900353), SCH772984, ulixertinib (BVD-523), CC-90003, GDC-0994 (RG-7482), ASN007, FR148083, 5-7-Oxozeaenol, 5-iodotubercidin, GDC0994, ONC201, or a combination thereof.

In some embodiments, the other PI3K inhibitor is another PI3Kα inhibitor. In some embodiments, the other PI3K inhibitor is a pan-PI3K inhibitor. In some embodiments, the other PI3K inhibitor is selected from buparlisib (BKM120), alpelisib (BYL719), WX-037, copanlisib (ALIQOPA™, BAY80-6946), dactolisib (NVP-BEZ235, BEZ-235), taselisib (GDC-0032, RG7604), sonolisib (PX-866), CUDC-907, PQR309, ZSTK474, SF1126, AZD8835, GDC-0077, ASN003, pictilisib (GDC-0941), pilaralisib (XL147, SAR245408), gedatolisib (PF-05212384, PKI-587), serabelisib (TAK-117, MLN1117, INK 1117), BGT-226 (NVP-BGT226), PF-04691502, apitolisib (GDC-0980), omipalisib (GSK2126458, GSK458), voxtalisib (XL756, SAR245409), AMG 511, CH5132799, GSK1059615, GDC-0084 (RG7666), VS-5584 (SB2343), PKI-402, wortmannin, LY294002, PI-103, rigosertib, XL-765, LY2023414, SAR260301, KIN-193 (AZD-6428), GS-9820, AMG319, GSK2636771, or a combination thereof.

In some embodiments, the AKT inhibitor is selected from miltefosine (IMPADIVO®), wortmannin, NL-71-101, H-89, GSK690693, CCT128930, AZD5363, ipatasertib (GDC-0068, RG7440), A-674563, A-443654, AT7867, AT13148, uprosertib, afuresertib, DC120, 2-[4-(2-aminoprop-2-yl)phenyl]-3-phenylquinoxaline, MK-2206, edelfosine, miltefosine, perifosine, erucylphophocholine, erufosine, SR13668, OSU-A9, PH-316, PHT-427, PIT-1, DM-PIT-1, triciribine (Triciribine Phosphate Monohydrate), API-1, N-(4-(5-(3-acetamidophenyl)-2-(2-aminopyridin-3-yl)-3H-imidazo[4,5-b]pyridin-3-yl)benzyl)-3-fluorobenzamide, ARQ092, BAY 1125976, 3-oxo-tirucallic acid, lactoquinomycin, boc-Phe-vinyl ketone, Perifosine (D-21266), TCN, TCN-P, GSK2141795, ONC201, or a combination thereof.

In some embodiments, the mTOR inhibitor is selected from MLN0128, vistusertib (AZD-2014), onatasertib (CC-223), CC-115, everolimus (RAD001), temsirolimus (CCI-779), ridaforolimus (AP-23573), sirolimus (rapamycin), ridaforolimus (MK-8669), or a combination thereof.

Non-limiting examples of farnesyl transferase inhibitors include lonafarnib, tipifarnib, BMS-214662, L778123, L744832, and FTI-277.

In some embodiments, a chemotherapeutic agent includes an anthracycline, cyclophosphamide, a taxane, a platinum-based agent, mitomycin, gemcitabine, eribulin (HALAVEN™), or combinations thereof.

Non-limiting examples of a taxane include paclitaxel, docetaxel, abraxane, and taxotere.

In some embodiments, the anthracycline is selected from daunorubicin, doxorubicin, epirubicin, idarubicin, and combinations thereof.

In some embodiments, the platinum-based agent is selected from carboplatin, cisplatin, oxaliplatin, nedplatin, triplatin tetranitrate, phenanthriplatin, picoplatin, satraplatin and combinations thereof.

Non-limiting examples of PARP inhibitors include olaparib (LYNPARZA®), talazoparib, rucaparib, niraparib, veliparib, BGB-290 (pamiparib), CEP 9722, E7016, iniparib, IMP4297, NOV1401, 2X-121, ABT-767, RBN-2397, BMN 673, KU-0059436 (AZD2281), BSI-201, PF-01367338, INO-1001, and JPI-289.

Non-limiting examples of aromatase inhibitors include aminoglutethimide, testolactone, anastrozole, letrozole, exemestane, vorozole, formestane, and fadrozole.

Non-limiting examples of selective estrogen receptor modulators or degraders (SERMs/SERDs) include tamoxifen, fulvestrant, brilanestrant, elacestrant, giredestrant, amcenestrant (SAR439859), AZD9833, rintodestrant, LSZ102, LY3484356, ZN-c5, D-0502, and SHR9549.

Non-limiting examples of immunotherapy include immune checkpoint therapies, atezolizumab (TECENTRIQ®), albumin-bound paclitaxel. Non-limiting examples of immune checkpoint therapies include inhibitors that target CTLA-4, PD-1, PD-L1, BTLA, LAG-3, A2AR, TIM-3, B7-H3, VISTA, IDO, and combinations thereof. In some embodimetnts the CTLA-4 inhibitor is ipilimumab (YERVOY®). In some embodiments, the PD-1 inhibitor is selected from pembrolizumab (KEYTRUDA®), nivolumab (OPDIVO®), cemiplimab (LIBTAYO®), or combinations thereof. In some embodiments, the PD-L1 inhibitor is selected from atezolizumab (TECENTRIQ®), avelumab (BAVENCIO®), durvalumab (IMFINZI®), or combinations thereof. In some embodiments, the LAG-3 inhibitor is IMP701 (LAG525). In some embodiments, the A2AR inhibitor is CPI-444. In some embodiments, the TIM-3 inhibitor is MBG453. In some embodiments, the B7-H3 inhibitor is enoblituzumab. In some embodiments, the VISTA inhibitor is JNJ-61610588. In some embodiments, the IDO inhibitor is indoximod. See, for example, Marin-Acevedo, et al., J Hematol Oncol. 11: 39 (2018).

In some embodiments, the additional therapy or therapeutic agent is selected from fulvestrant, capecitabine, trastuzumab, ado-trastuzumab emtansine, pertuzumab, paclitaxel, nab-paclitaxel, enzalutamide, olaparib, pegylated liposomal doxorubicin (PLD), trametinib, ribociclib, palbociclib, buparlisib, AEB071, everolimus, exemestane, cisplatin, letrozole, AMG 479, LSZ102, LEE011, cetuximab, AUY922, BGJ398, MEK162, LJM716, LGH447, imatinib, gemcitabine, LGX818, amcenestrant, and combinations thereof.

In some embodiments, additional therapeutic agents may also be administereted to treat potential side-effects for particular anticancer therapies and/or as palliative therapy, for example, opioids and corticosteroids. In some embodiments, the additional therapy or therapeutic agent described herein is selected from the group consisting of a glucagon-like peptide-1 (GLP-1) receptor agonist, a sodium-glucose transport protein 2 (SGLT-2) inhibitor, a dipeptidyl peptidase 4 (DPP-4) inhibitor, metformin, and combinations thereof.

Non-limiting examples of GLP-1 receptor agonists include liraglutide (VICTOZA®, NN2211), dulaglutide (LY2189265, TRULICITY®), exenatide (BYETTA®, BYDUREON®, Exendin-4), taspoglutide, lixisenatide (LYXUMIA®), albiglutide (TANZEUM®), semaglutide (OZEMPIC®), ZP2929, NNC0113-0987, BPI-3016, and TT401.

Non-limiting examples of SGLT-2 inhibitors include bexagliflozin, canagliflozin (INVOKANA®), dapagliflozin (FARXIGA®), empagliflozin (JARDIANCE®), ertugliflozin (STEGLATRO™), ipragliflozin (SUGLAT®), luseogliflozin (LUSEFI®), remogliflozin, serfliflozin, licofliglozin, sotagliflozin (ZYNQUISTA™), and tofogliflozin.

Non-limiting examples of DPP-4 inhibitors include, sitagliptin (JANUVIA®), vildagliptin, saxagliptin (ONGLYZA®), linagliptin (TRADJENDA®), gemigliptin, anagliptin, teneligliptin, alogliptin, trelagliptin (NESINA®), omarigliptin, evogliptin, and dutogliptin.

In some embodiments, the subject is also instructed to maintain a particular diet and/or exercise regimen to control blood sugar levels.

Accordingly, also provided herein is a method of treating cancer, comprising administering to a subject in need thereof a pharmaceutical combination for treating cancer which comprises (a) a compound of Formula (I), or a pharmaceutically acceptable salt thereof, (b) an additional therapeutic agent, and (c) optionally at least one pharmaceutically acceptable carrier for simultaneous, separate or sequential use for the treatment of cancer, wherein the amounts of the compound of Formula (I), or a pharmaceutically acceptable salt thereof, and the additional therapeutic agent are together effective in treating the cancer.

In some embodiments, the additional therapeutic agent(s) includes any one of the above listed therapies or therapeutic agents which are standards of care in cancers wherein the cancer has a dysregulation of a PIK3CA gene, a PI3Kα protein, or expression or activity, or level of any of the same.

These additional therapeutic agents may be administered with one or more doses of the compound of Formula (I), or a pharmaceutically acceptable salt thereof, or pharmaceutical composition thereof, as part of the same or separate dosage forms, via the same or different routes of administration, and/or on the same or different administration schedules according to standard pharmaceutical practice known to one skilled in the art.

Also provided herein is (i) a pharmaceutical combination for treating a cancer in a subject in need thereof, which comprises (a) a compound of Formula (I), or a pharmaceutically acceptable salt thereof, (b) at least one additional therapeutic agent (e.g., any of the exemplary additional therapeutic agents described herein or known in the art), and (c) optionally at least one pharmaceutically acceptable carrier for simultaneous, separate or sequential use for the treatment of cancer, wherein the amounts of the compound of Formula (I), or pharmaceutically acceptable salt thereof, and of the additional therapeutic agent are together effective in treating the cancer; (ii) a pharmaceutical composition comprising such a combination; (iii) the use of such a combination for the preparation of a medicament for the treatment of cancer; and (iv) a commercial package or product comprising such a combination as a combined preparation for simultaneous, separate or sequential use; and to a method of treatment of cancer in a subject in need thereof. In some embodiments, the cancer is a PI3Kα-associated cancer.

The term “pharmaceutical combination”, as used herein, refers to a pharmaceutical therapy resulting from the mixing or combining of more than one active ingredient and includes both fixed and non-fixed combinations of the active ingredients. The term “fixed combination” means that a compound of Formula (I), or a pharmaceutically acceptable salt thereof, and at least one additional therapeutic agent (e.g., a chemotherapeutic agent), are both administered to a subject simultaneously in the form of a single composition or dosage. The term “non-fixed combination” means that a compound of Formula (I), or a pharmaceutically acceptable salt thereof, and at least one additional therapeutic agent (e.g., chemotherapeutic agent) are formulated as separate compositions or dosages such that they may be administered to a subject in need thereof simultaneously, concurrently or sequentially with variable intervening time limits, wherein such administration provides effective levels of the two or more compounds in the body of the subject.

These also apply to cocktail therapies, e.g., the administration of three or more active ingredients Accordingly, also provided herein is a method of treating a cancer, comprising administering to a subject in need thereof a pharmaceutical combination for treating cancer which comprises (a) a compound of Formula (I), or pharmaceutically acceptable salt thereof, and (b) an additional therapeutic agent, wherein the compound of Formula (I) and the additional therapeutic agent are administered simultaneously, separately or sequentially, wherein the amounts of the compound of Formula (I), or pharmaceutically acceptable salt thereof, and the additional therapeutic agent are together effective in treating the cancer. In some embodiments, the compound of Formula (I), or pharmaceutically acceptable salt thereof, and the additional therapeutic agent are administered simultaneously as separate dosages. In some embodiments, the compound of Formula (I), or pharmaceutically acceptable salt thereof, and the additional therapeutic agent are administered as separate dosages sequentially in any order, in jointly therapeutically effective amounts, e.g., in daily or intermittently dosages. In some embodiments, the compound of Formula (I), or pharmaceutically acceptable salt thereof, and the additional therapeutic agent are administered simultaneously as a combined dosage.

### Embodiments

Embodiment 1: A compound of Formula (I):

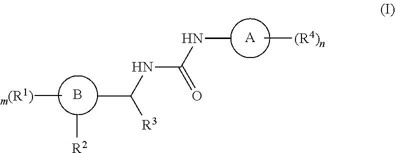

or a pharmaceutically acceptable salt thereof, wherein:
Ring B is a 9-membered heteroaryl group, wherein Ring B is not 2-benzofuranyl or 2-indolyl;
each R¹ is independently selected from halogen, hydroxyl, cyano, C1-C6 alkyl optionally substituted with hydroxyl, and C3-C6 cycloalkyl;
m is 0, 1, 2, or 3;
R² is halogen, hydroxyl, C1-C6 alkyl optionally substituted with hydroxyl, C1-C6 haloalkyl, or C3-C6 cycloalkyl optionally substituted with 1 or 2 fluoro;
R³ is a C1-C6 alkyl, a C1-C6 haloalkyl, or a C3-C6 cycloalkyl optionally substituted with 1 or 2 substituents independently selected from fluoro and C1-C6 alkyl;
Ring A is a 6-10 membered aryl, a C3-C8 cycloalkyl, a 5-10 membered heteroaryl, or a 4-10 membered heterocyclyl;
each R⁴ is independently selected from the group consisting of:
(i) halogen,
(ii) C1-C6 alkyl optionally substituted with 1 or 2 hydroxyl or —NR^{A}R^{B},
(iii) C1-C6 alkoxy optionally substituted with 1-2 substituents independently selected from hydroxyl and C3-C6 cycloalkyl,
(iv) C1-C6 haloalkyl,
(v) hydroxyl,
(vi) cyano,
(vii) —CO₂H,
(viii) —NR^{A}R^{B},
(ix) ═NR^{A2},
(x) —C(═O)NR^{C}R^{D},
(xi) —SO₂(NR^{E}R^{F}),
(xii) —SO₂(C1-C6 alkyl),
(xiii) —S(═O)(═NH)(C1-C6 alkyl),
(xiv) —C(═O)(C1-C6 alkyl),
(xv) —CO₂(C1-C6 alkyl),
(xvi) 5-6 membered heteroaryl optionally substituted with C1-C6 alkyl,
(xvii) 3-9 membered heterocyclyl optionally substituted with 1 or 2 independently selected R^{G}, and
(xviii) C3-C6 cycloalkyl optionally substituted with 1 or 2 independently selected R^{G};
n is 0, 1, or 2;
each R^{A}, R^{A1}, R^{B}, R^{B1}, R^{C}, R^{C1}, R^{D}, R^{D1}, R^{E}, and R^{F} is independently
(i) hydrogen,
(ii) hydroxyl,
(iii) 4-6 membered heterocyclyl,
(iv) C1-C6 haloalkyl,
(v) —C(═O)(C1-C6 alkyl),
(vi) —C(═O)O(C1-C6 alkyl),
(vii) —SO₂(C1-C6 alkyl),
(viii) C3-C6 cycloalkyl optionally substituted with hydroxyl, or
(ix) C1-C6 alkyl optionally substituted with 1-2 substituents independently selected from hydroxyl, —C(═O)NR^{B2}R^{C2}, 5-6 membered heteroaryl, C3-C6 cycloalkyl, —SO₂(C1-C6 alkyl), —CO₂H, and —SO₂(NH₂); or
R^{C} and R^{D}, together with the nitrogen atom to which they are attached form a 4-10 membered heterocyclyl optionally substituted with 1-2 substituents independently selected from hydroxyl, halogen, —C(═O)NR^{B1}R^{C1}, —SO₂(C1-C6 alkyl), —CO₂H, C1-C6 alkyl optionally substituted with hydroxyl, C1-C6 alkoxy, and C1-C6 haloalkoxy;
each R^{A2}, R^{B2}, and R^{C2} is independently hydrogen or C1-C6 alkyl;
each R^{G} is independently selected from the group consisting of: fluoro, cyano, hydroxyl, C1-C6 alkyl optionally substituted with hydroxyl, C1-C6 alkoxy, —NR^{A1}R^{B1}, ═NR^{A2}, —C(═O)NR^{C1}R^{D1}, —CO₂(C1-C6 alkyl), C1-C6 haloalkyl, C3-C6 cycloalkyl, C1-C6 haloalkoxy, —SO₂(C1-C6 alkyl), and —CO₂H.

Embodiment 2: The compound of embodiment 1, wherein

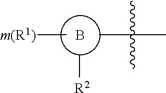

is

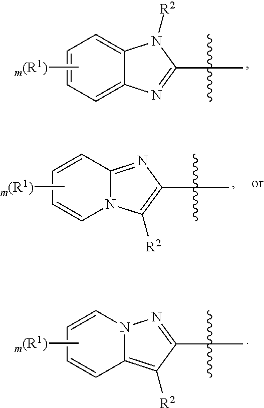

Embodiment 3: The compound of embodiment 2, wherein

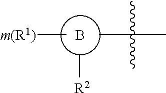

is

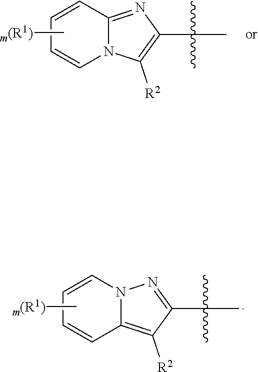

Embodiment 4: The compound of embodiment 2, wherein

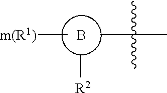

is

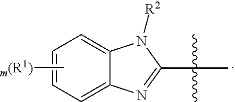

Embodiment 5: The compound of embodiment 2, wherein

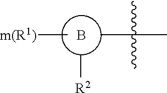

is

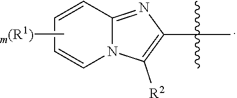

Embodiment 6: The compound of embodiment 2, wherein

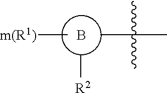

is

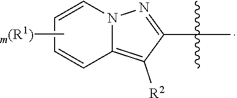

Embodiment 7: The compound of any one of embodiments 1-6, wherein m is 1.

Embodiment 8: The compound of any one of embodiments 1-6, wherein m is 2.

Embodiment 9: The compound of any one of embodiments 1-8, wherein each R¹ is halogen.

Embodiment 10: The compound of any one of embodiments 1-9, wherein each R¹ is selected from fluoro and chloro.

Embodiment 11: The compound of any one of embodiments 1-10, wherein each R¹ is fluoro.

Embodiment 12: The compound of any one of embodiments 1-8, wherein each R¹ is hydroxyl.

Embodiment 13: The compound of any one of embodiments 1-8, wherein one R¹ is cyano. Embodiment 14: The compound of any one of embodiments 1-8, wherein one R¹ is C1-C6 alkyl optionally substituted with hydroxyl.

Embodiment 15: The compound of any one of embodiments 1-8, wherein one R¹ is C3-C6 cycloalkyl.

Embodiment 16: The compound of any one of embodiments 1-6, wherein m is 0.

Embodiment 17: The compound of any one of embodiments 1-16, wherein R² is a C1-C6 alkyl optionally substituted with hydroxyl.

Embodiment 18: The compound of any one of embodiments 1-17, wherein R² is a unsubstituted C1-C6 alkyl.

Embodiment 19: The compound of embodiment 18, wherein R² is methyl.

Embodiment 20: The compound of any one of embodiments 1-16, wherein R² is a C1-C6 haloalkyl.

Embodiment 21: The compound of embodiment 20, wherein R² is difluoromethyl.

Embodiment 22: The compound of embodiment 20, wherein R² is trifluoromethyl.

Embodiment 23: The compound of any one of embodiments 1-16, wherein R² is halogen. Embodiment 24: The compound of any one of embodiments 1-16, wherein R² is hydroxyl. Embodiment 25: The compound of any one of embodiments 1-16, wherein R² is C3-C6 cycloalkyl optionally substituted with 1 or 2 fluoro.

Embodiment 26: The compound of any one of embodiments 1-25, wherein R³ is a C1-C6 haloalkyl.

Embodiment 27: The compound of any one of embodiments 1-26, wherein R³ is difluoromethyl.

Embodiment 28: The compound of any one of embodiments 1-26, wherein R³ is trifluoromethyl.

Embodiment 29: The compound of any one of embodiments 1-25, wherein R³ is a C1-C6 alkyl.

Embodiment 30: The compound of any one of embodiments 1-25, and 29, wherein R³ is Me, Et, or iPr.

Embodiment 31: The compound of any one of embodiments 1-25, wherein R³ is C3-C6 cycloalkyl optionally substituted with 1 or 2 substituents independently selected from fluoro and C1-C6 alkyl.

Embodiment 32: The compound of any one of embodiments 1-31, wherein Ring A is a 5-10 membered heteroaryl.

Embodiment 33: The compound of any one of embodiments 1-32, wherein Ring A is a 5-6 membered heteroaryl.

Embodiment 34: The compound of any one of embodiments 1-33, wherein Ring A is pyrimidinyl, pyridyl, thiazolyl, thiophenyl, or pyrazolyl.

Embodiment 35: The compound of any one of embodiments 1-34, wherein Ring A is pyrimidinyl.

Embodiment 36: The compound of any one of embodiments 1-34, wherein Ring A is pyridyl.

Embodiment 37: The compound of any one of embodiments 1-34, wherein Ring A is thiazolyl.

Embodiment 38: The compound of any one of embodiments 1-34, wherein Ring A is thiophenyl.

Embodiment 39: The compound of any one of embodiments 1-34, wherein Ring A is pyrazolyl.

Embodiment 40: The compound of any one of embodiments 1-32, wherein Ring A is a 9-10 membered heteroaryl.

Embodiment 41: The compound of any one of embodiments 1-32 and 40, wherein Ring A is benzimidazolyl, indazolyl, indolyl, quinazolone, isobenzofuranonyl, isoindolinonyl, or imidazo[1,2-a]pyridinyl.

Embodiment 42: The compound of any one of embodiments 1-32 and 40-41, wherein Ring A is benzimidazolyl.

Embodiment 43: The compound of any one of embodiments 1-32 and 40-41, wherein Ring A is indazolyl.

Embodiment 44: The compound of any one of embodiments 1-32 and 40-41, wherein Ring A is indolyl.

Embodiment 45: The compound of any one of embodiments 1-32 and 40-41, wherein Ring A is quinazolone.

Embodiment 46: The compound of any one of embodiments 1-32 and 40-41, wherein Ring A is isobenzofuranonyl.

Embodiment 47: The compound of any one of embodiments 1-32 and 40-41, wherein Ring A is isoindolinonyl.

Embodiment 48: The compound of any one of embodiments 1-32 and 40-41, wherein Ring A is imidazo[1,2-a]pyridinyl.

Embodiment 49: The compound of any one of embodiments 1-31, wherein Ring A is 6-10 membered aryl.

Embodiment 50: The compound of any one of embodiments 1-31 and 49, wherein Ring A is phenyl.

Embodiment 51: The compound of any one of embodiments 1-31, wherein Ring A is a C3-C8 cycloalkyl.

Embodiment 52: The compound of any one of embodiments 1-31, wherein Ring A is a 4-10 membered heterocyclyl.

Embodiment 53: The compound of any one of embodiments 1-31 and 52, wherein Ring A is a 4-6 membered heterocyclyl.

Embodiment 54: The compound of any one of embodiments 1-53, wherein n is 1.

Embodiment 55: The compound of any one of embodiments 1-53, wherein n is 2.

Embodiment 56: The compound of any one of embodiments 1-55, wherein one R⁴ is C1-C6 alkoxy optionally substituted with 1-2 substituents independently selected from hydroxyl and C3-C6 cycloalkyl.

Embodiment 57: The compound of any one of embodiments 1-55, wherein one R⁴ is C1-C6 haloalkyl.

Embodiment 58: The compound of any one of embodiments 1-55, wherein one R⁴ is hydroxyl, cyano, —CO₂H, halogen, or C1-C6 alkyl optionally substituted with 1-2 hydroxyl or —NR^{A}R^{B}.

Embodiment 59: The compound of any one of embodiments 1-55, wherein one R⁴ is —NR^{A}R^{B}, ═NR^{A2}, —C(═O)NR^{C}R^{D}, —SO₂(NR^{E}R^{F}), —SO₂(C1-C6 alkyl), —S(═O)(═NH)(C1-C6 alkyl), —C(═O)(C1-C6 alkyl), or —CO₂(C1-C6 alkyl).

Embodiment 60: The compound of any one of embodiments 1-55, wherein one R⁴ is 5-6 membered heteroaryl optionally substituted with C1-C6 alkyl.

Embodiment 6l: The compound of any one of embodiments 1-55, wherein one R⁴ is 3-9 membered heterocyclyl optionally substituted with 1 or 2 independently selected R^{G}.

Embodiment 62: The compound of any one of embodiments 1-55, wherein one R⁴ is C3-C6 cycloalkyl optionally substituted with 1 or 2 independently selected R^{G}.

Embodiment 63: The compound of any one of embodiments 1-53, wherein n is 0.

Embodiment 64: A compound selected from the group consisting of the compounds in Table A or a pharmaceutically acceptable salt thereof.

Embodiment 65: A pharmaceutical composition comprising a compound of any one of embodiments 1-64, or a pharmaceutically acceptable salt thereof, and one or more pharmaceutically acceptable excipients.

Embodiment 66: A method for treating cancer in a subject in need thereof, the method comprising administering to the subject a therapeutically effective amount of a compound of any one of embodiments 1-64, or a pharmaceutically acceptable salt thereof, or a pharmaceutical composition of embodiment 65.

Embodiment 67: A method for treating cancer in a subject in need thereof, the method comprising (a) determining that the cancer is associated with a dysregulation of a PIK3CA gene, a PI3Kα protein, or expression or activity or level of any of the same; and (b) administering to the subject a therapeutically effective amount of a compound of any one of embodiments 1-64, or a pharmaceutically acceptable salt thereof, or a pharmaceutical composition of embodiment 65.

Embodiment 68: A method of treating a PI3Kα-associated cancer in a subject, the method comprising administering to a subject identified or diagnosed as having a PI3Kα-associated cancer a therapeutically effective amount of a compound of any one of embodiments 1-64 or a pharmaceutically acceptable salt thereof, or a pharmaceutical composition of embodiment 65.

Embodiment 69: A method for modulating PI3Kα in a mammalian cell, the method comprising contacting the mammalian cell with an effective amount of a compound of any one of embodiments 1-64, or a pharmaceutically acceptable salt thereof.

### EXAMPLES

### Compound Preparation

The compounds disclosed herein can be prepared in a variety of ways using commercially available starting materials, compounds known in the literature, or from readily prepared intermediates, by employing standard synthetic methods and procedures either known to those skilled in the art, or in light of the teachings herein. The synthesis of the compounds disclosed herein can be achieved by generally following the schemes provided herein, with modification for specific desired substituents.

Standard synthetic methods and procedures for the preparation of organic molecules and functional group transformations and manipulations can be obtained from the relevant scientific literature or from standard textbooks in the field. Although not limited to any one or several sources, classic texts such as R. Larock, Comprehensive Organic Transformations, VCH Publishers (1989); L. Fieser and M. Fieser, Fieser and Fieser's Reagents for Organic Synthesis, John Wiley and Sons (1994); Smith, M. B., March, J., March's Advanced Organic Chemistry: Reactions, Mechanisms, and Structure, 5th edition, John Wiley & Sons: New York, 2001; and Greene, T. W., Wuts, P. G. M., Protective Groups in Organic Synthesis, 3rd edition, John Wiley & Sons: New York, 1999, are useful and recognized reference textbooks of organic synthesis known to those in the art. The following descriptions of synthetic methods are designed to illustrate, but not to limit, general procedures for the preparation of compounds of the present disclosure.

The synthetic processes disclosed herein can tolerate a wide variety of functional groups; therefore, various substituted starting materials can be used. The processes generally provide the desired final compound at or near the end of the overall process, although it may be desirable in certain instances to further convert the compound to a pharmaceutically acceptable salt thereof.

### Example 1: Preparation of Compound 1

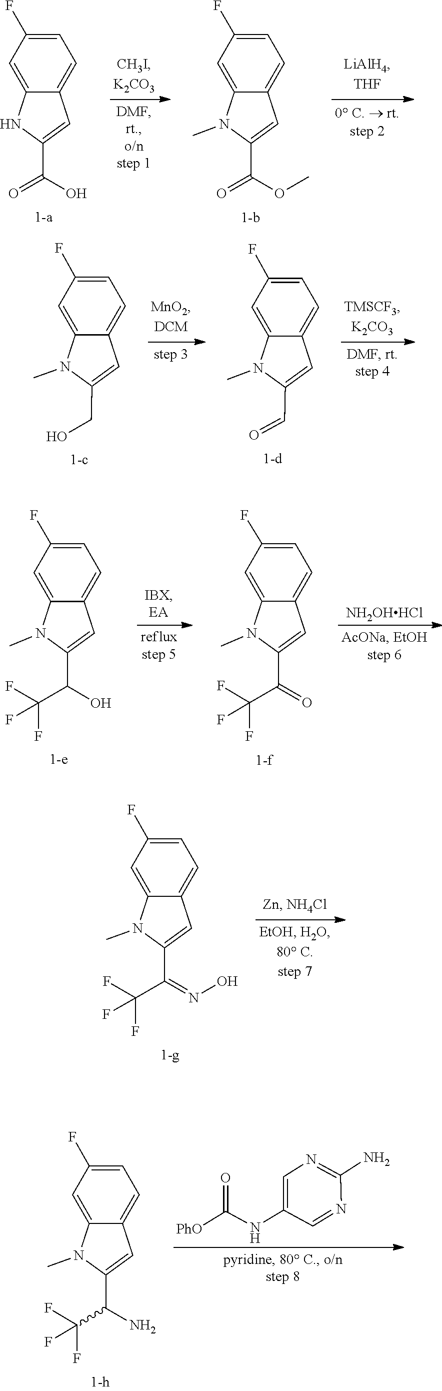

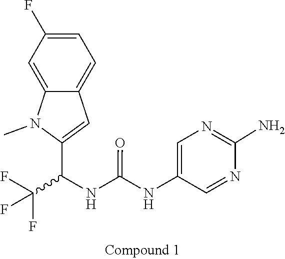 [SMILES: Cn1c(C(NC(=O)Nc2cnc(N)nc2)C(F)(F)F)cc2ccc(F)cc21]

### Step 1

A solution of 6-fluoro-1H-indole-2-carboxylic acid (1-a; 2.00 g, 11.16 mmol, 1.00 equiv) in DMF (14 mL) was treated with K₂CO₃ (4.63 g, 33.49 mmol, 3.00 equiv) for 1 min at 0° C. under nitrogen atmosphere followed by the addition of CH₃I (2.78 mL, 44.66 mmol, 4.00 equiv) dropwise at 0° C. The solution was stirred for 72 h at room temperature under nitrogen atmosphere. The reaction was quenched with sat. NH₄Cl (aq.) at 0° C. The precipitated solids were collected by filtration and washed with water (1×200 mL) to afford methyl 6-fluoro-1-methylindole-2-carboxylate (1-b; 2 g, 87%) as an off-white solid. MS (ESI): mass calcd. for C₁₁H₁₀FNO₂, 207.07, m/z found 208.05[M+H]⁺.

### Step 2

To a stirred solution of methyl 6-fluoro-1-methylindole-2-carboxylate (1-b; 1.00 g, 4.83 mmol, 1.00 equiv) in THF (4 mL) was added a solution of LiAlH₄ in THF (5.79 mL, 5.79 mmol, 1.20 equiv) dropwise at 0° C. under nitrogen atmosphere. The solution was stirred for 1 h at room temperature under nitrogen atmosphere. The reaction was monitored by LCMS. The reaction was quenched with sodium sulfate decahydrate at 0° C. The resulting mixture was filtered, the filter cake was washed with ethyl acetate (1×100 mL). The filtrate was concentrated under reduced pressure to afford (6-fluoro-1-methylindol-2-yl) methanol (1-c; 875 mg) as a brown solid. The crude product was used in the next step directly without further purification. MS (ESI): mass calcd. for C₁₀H₁₀FNO, 179.07, m/z found 180.05 [M+H]⁺.

### Step 3

To a stirred solution of (6-fluoro-1-methylindol-2-yl)methanol (1-c; 775 mg, 4.32 mmol, 1.00 equiv) in DCM (8 mL) was added manganese dioxide (3.76 g, 43.25 mmol, 10.00 equiv). The solution was stirred for 1 h at room temperature under nitrogen atmosphere. The reaction was monitored by LCMS. The resulting mixture was filtered, the filter cake was washed with ethyl acetate (1×100 mL). The filtrate was concentrated under reduced pressure to afford 6-fluoro-1-methylindole-2-carbaldehyde (1-d; 754 mg) as a yellow solid. The crude product was used in the next step directly without further purification. MS (ESI): mass calcd. for C₁₀H₈FNO, 177.06, m/z found 178.05 [M+H]⁺.

### Step 4

To a stirred solution of 6-fluoro-1-methylindole-2-carbaldehyde (1-d; 650 mg, 3.67 mmol, 1.00 equiv) and K₂CO₃ (1.52 g, 11.00 mmol, 3.00 equiv) in DMF (7 mL) was added TMSCF₃ (1.04 g, 7.34 mmol, 2.00 equiv) dropwise at 0° C. under nitrogen atmosphere. The resulting mixture was stirred for 5 h at room temperature under nitrogen atmosphere. The reaction was monitored by LCMS. The resulting mixture was diluted with water (150 mL). The resulting mixture was extracted with EtOAc (3×100 mL). The combined organic layers were washed with brine (1×150 mL), dried over anhydrous Na₂SO₄. After filtration, the filtrate was concentrated under reduced pressure. The residue was purified by silica gel column chromatography, eluted with PE/EA (10:1) to afford 2,2,2-trifluoro-1-(6-fluoro-1-methylindol-2-yl)ethanol (1-e; 743 mg, 82%) as a brown solid. MS (ESI): mass calcd. for C₁₁H₉F₄NO, 247.06, m/z found 248.05 [M+H]⁺.

### Step 5

To a stirred solution of 2,2,2-trifluoro-1-(6-fluoro-1-methylindol-2-yl)ethanol (1-e; 600 mg, 2.43 mmol, 1.00 equiv) in EA (6 mL) was added IBX (1.36 g, 4.85 mmol, 2.00 equiv) in portions at 0° C. under nitrogen atmosphere. The resulting mixture was stirred for overnight at 80° C. under nitrogen atmosphere. The reaction was monitored by TLC. The resulting mixture was filtered, the filter cake was washed with PE (1×60 mL). The filtrate was concentrated under reduced pressure. The residue was purified by silica gel column chromatography, eluted with PE to afford 2,2,2-trifluoro-1-(6-fluoro-1-methylindol-2-yl)ethanone (1-f; 560 mg, 94%) as a white solid. MS MS (ESI): mass calcd. for C₁₁H₇F₄NO, 245.05, m/z found 245.90 [M+H]⁺.

### Step 6

A solution of 2,2,2-trifluoro-1-(6-fluoro-1-methylindol-2-yl)ethanone (1-f; 560 mg, 2.28 mmol, 1.00 equiv) in EtOH (6 mL) was treated with AcONa (937 mg, 11.42 mmol, 5.00 equiv) for 1 min at 0° C. under nitrogen atmosphere followed by the addition of NH₂OH·HCl (794 mg, 11.42 mmol, 5.00 equiv) in portions at 0° C. The solution was stirred overnight at 80° C. under nitrogen atmosphere. The reaction was monitored by LCMS. The resulting mixture was diluted with water (100 mL). The resulting mixture was extracted with EtOAc (3×50 mL). The combined organic layers were washed with brine (1×100 mL), dried over anhydrous Na₂SO₄. After filtration, the filtrate was concentrated under reduced pressure. The residue was purified by silica gel column chromatography, eluted with PE/EA (10:1) to afford (E)-N-[2,2,2-trifluoro-1-(6-fluoro-1-methylindol-2-yl)ethylidene]hydroxylamine (1-g; 460 mg, 77%) as a white solid. MS (ESI): mass calcd. for C₁₁H₈F₄N₂O, 260.06, m/z found 258.90[M−H]⁻.

### Step 7

A solution of (E)-N-[2,2,2-trifluoro-1-(6-fluoro-1-methylindol-2-yl)ethylidene]hydroxylamine (1-g; 250 mg, 0.96 mmol, 1.00 equiv), Zn powder (628 mg, 9.61 mmol, 10.00 equiv) and NH₄Cl (514 mg, 9.61 mmol, 10.00 equiv) in EtOH (2 mL) and H₂O (0.4 mL) was stirred for overnight at 80° C. under nitrogen atmosphere. The reaction was monitored by LCMS. The resulting mixture was filtered, the filter cake was washed with EtOAc (1×20 mL). The filtrate was concentrated under reduced pressure. The residue was purified by silica gel column chromatography, eluted with PE/EA (10:1) to afford 2,2,2-trifluoro-1-(6-fluoro-1-methylindol-2-yl)ethanamine (1-h; 143 mg, 60%) as a brown solid. MS (ESI): mass calcd. for C₁₁H₁₀F₄N₂, 246.08, m/z found 230.05 [M-NH₃+H]⁺.

### Step 8

To a stirred solution of 2,2,2-trifluoro-1-(6-fluoro-1-methylindol-2-yl)ethanamine (1-h; 140 mg, 0.57 mmol, 1.00 equiv) in pyridine (1 mL) was added phenyl N-(2-aminopyrimidin-5-yl)carbamate (157 mg, 0.68 mmol, 1.20 equiv). The solution was stirred overnight at 80° C. under nitrogen atmosphere. The reaction was monitored by LCMS. The resulting mixture was diluted with water (15 mL). The resulting mixture was extracted with EtOAc (3×10 mL). The combined organic layers were washed with brine (1×15 mL), dried over anhydrous Na₂SO₄. After filtration, the filtrate was concentrated under reduced pressure. The residue was purified by silica gel column chromatography, eluting with CH₂Cl₂/MeOH (10:1) to afford 1-(2-aminopyrimidin-5-yl)-3-[2,2,2-trifluoro-1-(6-fluoro-1-methylindol-2-yl)ethyl]urea (170 mg) as a light yellow solid. The crude product (170 mg) was purified by reverse phase flash with the following conditions (column, C18 silica gel; mobile phase, MeCN in Water (10 mmol/L NH₄HCO₃), 10% to 50% gradient in 30 min; detector, UV 254 nm) to afford 1-(2-aminopyrimidin-5-yl)-3-[2,2,2-trifluoro-1-(6-fluoro-1-methylindol-2-yl)ethyl]urea (Compound 1; 60 mg, 28%) as a light yellow solid. MS (ESI): mass calcd. for C16H₁₄F₄N₆O, 382.12, m/z found 383.15[M+H]⁺ 0.1H NMR (400 MHz, DMSO-d6) δ 8.22 (s, 2H), 8.07 (s, 1H), 7.60 (dd, J=8.7, 5.5 Hz, 1H), 7.53 (d, J=9.3 Hz, 1H), 7.41-7.37 (m, 1H), 6.96-6.91 (m, 1H), 6.66 (s, 1H), 6.40 (s, 2H), 5.99 (p, J=8.0 Hz, 1H), 3.73 (s, 3H).

### Example 2: Preparation of Compound 2

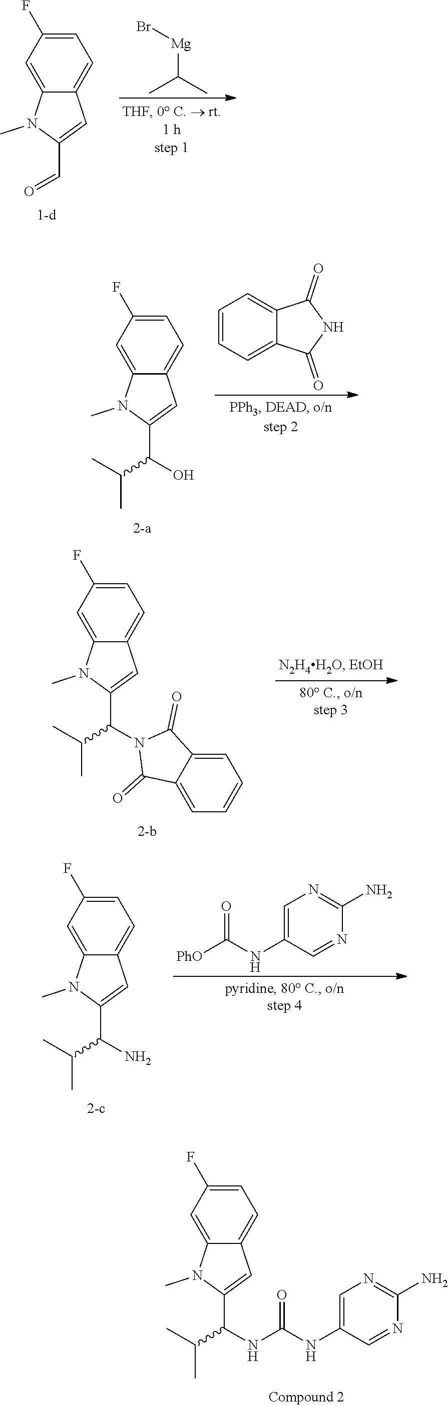

### Step 1

To a stirred solution of 6-fluoro-1-methylindole-2-carbaldehyde (1-d; 790 mg, 4.46 mmol, 1.00 equiv) in THF (16 mL) was added isopropylmagnesium bromide, 1 M solution in THF (22 mL, 22.30 mmol, 5.00 equiv) dropwise at 0° C. under nitrogen atmosphere. The resulting mixture was stirred for 1h at room temperature under nitrogen atmosphere. The reaction was quenched with sat. NH₄Cl (aq.) at 0° C. The resulting mixture was extracted with EtOAc (3×100 mL). The combined organic layers were washed with water (1×100 mL), dried over anhydrous Na₂SO₄. After filtration, the filtrate was concentrated under reduced pressure. The residue was purified by silica gel column chromatography, eluted with PE/EA (10:1) to afford 1-(6-fluoro-1-methylindol-2-yl)-2-methylpropan-1-ol (2-a; 580 mg, 59%) as a green solid. MS (ESI): mass calcd. for C₁₃H₁₆FNO, 221.12, m/z found 222.20 [M+H]⁺.

### Step 2

To a stirred solution of 1-(6-fluoro-1-methylindol-2-yl)-2-methylpropan-1-ol (2-a; 300 mg, 1.36 mmol, 1.00 equiv) and phthalimide (219 mg, 1.49 mmol, 1.10 equiv) in THF (3 mL) were added PPh₃ (533 mg, 2.03 mmol, 1.50 equiv) and DEAD (354 mg, 2.03 mmol, 1.50 equiv) in portions at 0° C. under nitrogen atmosphere. The resulting mixture was stirred overnight at room temperature under nitrogen atmosphere. The resulting mixture was diluted with water (10 mL) and extracted with EtOAc (3×50 mL). The combined organic layers were washed with water (1×30 mL), dried over anhydrous Na₂SO₄. After filtration, the filtrate was concentrated under reduced pressure. The residue was purified by silica gel column chromatography, eluted with PE/EA (2:1) to afford 2-[1-(6-fluoro-1-methylindol-2-yl)-2-methylpropyl]isoindole-1,3-dione (2-b; 330 mg, 69%) as a yellow oil. MS (ESI): mass calcd. for C₂₁H₁₉FN₂O₂, 350.14, m/z found 351.10 [M+H]⁺.

### Step 3

To a stirred solution of 2-[1-(6-fluoro-1-methylindol-2-yl)-2-methylpropyl]isoindole-1,3-dione (2-b; 280 mg, 0.80 mmol, 1.00 equiv) in EtOH (5 mL) was added NH₂NH₂·H₂O (400 mg, 7.99 mmol, 10.00 equiv). The resulting mixture was stirred for 2 h at 80° C. under air atmosphere. The resulting mixture was diluted with water (20 mL) and extracted with EtOAc (3×20 mL). The combined organic layers were washed with 1M NaOH (1×20 mL), water (1×20 mL), brine (1×20 mL), dried over anhydrous Na₂SO₄. After filtration, the filtrate was concentrated under reduced pressure. The residue was purified by silica gel column chromatography, eluted with CH₂C1₂/MeOH (10:1) to afford 1-(6-fluoro-1-methylindol-2-yl)-2-methylpropan-1-amine (2-c; 170 mg, 96%) as a yellow oil. MS (ESI): mass calcd. for C₁₃H₁₇FN₂, 220.14, m/z found 221.15 [M+H]⁺.

### Step 4

A solution of 1-(6-fluoro-1-methylindol-2-yl)-2-methylpropan-1-amine (2-c; 100 mg, 0.45 mmol, 1.00 equiv) and phenyl N-(2-aminopyrimidin-5-yl)carbamate (125 mg, 0.54 mmol, 1.20 equiv) in pyridine (2 mL) was stirred overnight at 80° C. under nitrogen atmosphere. The reaction was monitored by LCMS. The resulting mixture was diluted with water (10 mL) and extracted with EtOAc (3×20 mL). The combined organic layers was washed with 1M HCl (1×20 mL). The resulting mixture was concentrated under reduced pressure. The residue was purified by reverse flash chromatography with the following conditions: column, C18 silica gel; mobile phase, MeCN in Water (0.1% FA), 10% to 60% gradient in 30 min; detector, UV 254 nm to afford 1-(2-aminopyrimidin-5-yl)-3-[1-(6-fluoro-1-methylindol-2-yl)-2-methylpropyl]urea (Compound 2; 54.3 mg, 34%) as a light yellow solid. MS (ESI): mass calcd. for C₁₈H₂₁FN₆O, 356.18, m/z found 357.05[M+H]⁺. ¹H NMR (400 MHz, DMSO-d₆) δ 8.20 (s, 2H), 8.09 (s, 1H), 7.47 (dd, J=8.6, 5.5 Hz, 1H), 7.30-7.27 (m, 1H), 6.87-6.81 (m, 1H), 6.73 (d, J=9.0 Hz, 1H), 6.36 (s, 1H), 6.26 (s, 2H), 4.79-4.75 (m, 1H), 3.70 (s, 3H), 2.16-2.08 (m, 1H), 0.97 (d, J=6.6 Hz, 3H), 0.93 (d, J=6.6 Hz, 3H).

### Example 3: Preparation of Compound 3

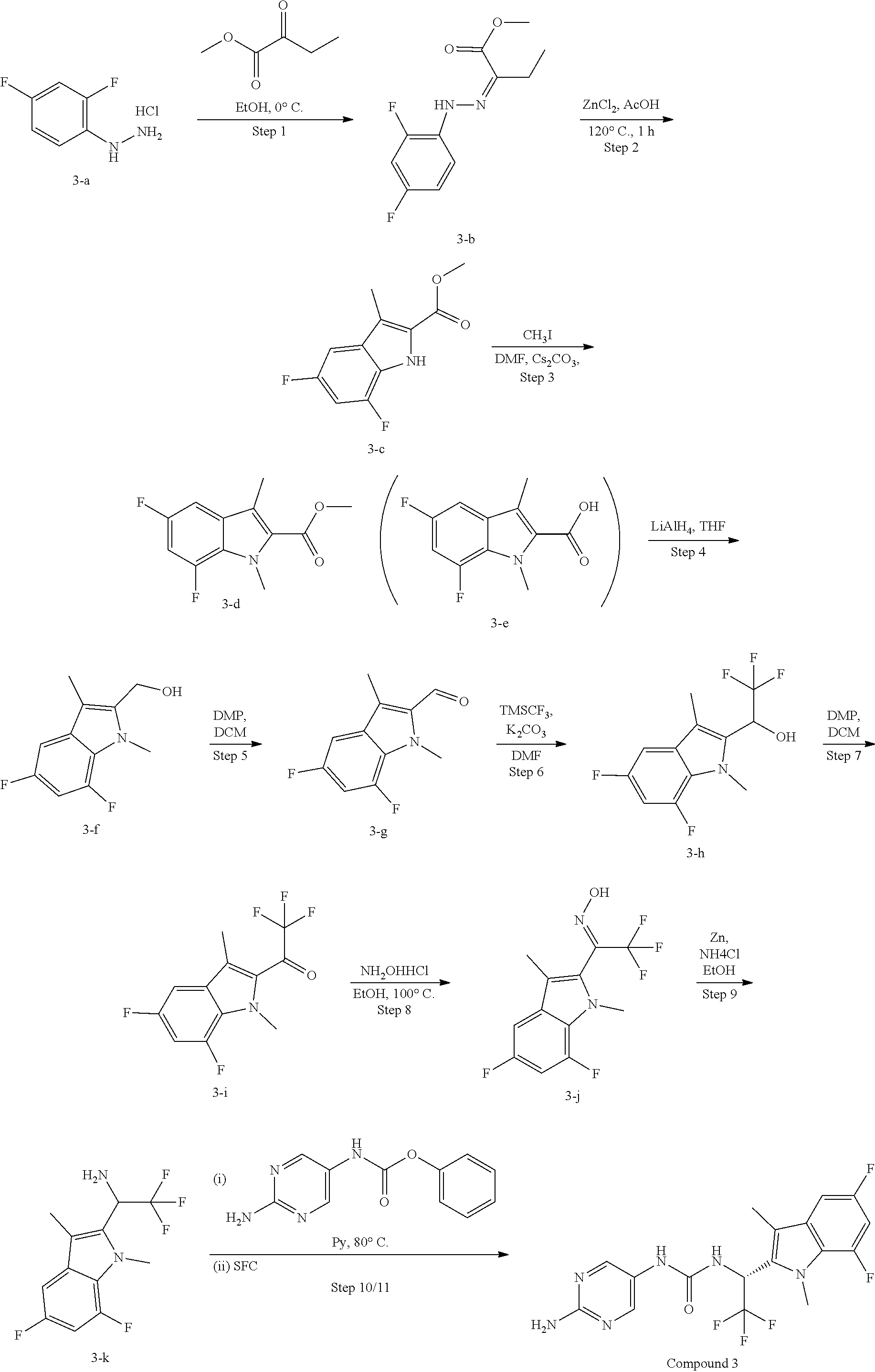

### Step 1

A solution of (2,4-difluorophenyl)hydrazine (3-a; 1.8 g, 12.49 mmol, 1 equiv) and dimethylpyruvic acid (1.89 g, 16.28 mmol, 1.30 equiv) in EtOH (60 mL) was stirred for 3 h at room temperature. After the reaction was completed, the solvent was evaporated to get methyl (2Z)-2-[2-(2,4-difluorophenyl)hydrazin-1-ylidene]butanoate (3-b; 2.43 g, 80.33%) yellow semi-solid as product.

### Step 2

A solution of methyl (2Z)-2-[2-(2,4-difluorophenyl)hydrazin-1-ylidene]butanoate (3-b; 2.43 g, 10.00 mmol, 1 equiv) and ZnCl₂ (50.6 g, 371.6 mmol, 5 equiv) in AcOH was stirred for 1 h at 120° C. After the reaction was completed, the pH of the solution was adjusted with NaHCO₃to 8, then the reaction mixture was extracted with EA, washed with the brine, and dried over anhydrous Na₂SO₄ to afford methyl 5,7-difluoro-3-methyl-1H-indole-2-carboxylate (3-c; 5 g, 30%) yellow solid as product.

### Step 3

A solution of methyl 5,7-difluoro-3-methyl-1H-indole-2-carboxylate (3-c; 1.55 g, 6.88 mmol, 1 equiv), CH₃I (4.88 g, 34.38 mmol, 5.00 equiv) and Cs₂CO₃ (5.61 g, 17.21 mmol, 2.5 equiv) in DMF (50 mL) was stirred overnight at room temperature. After the reaction was completed, the mixture was quenched with water, extracted with EA, washed with the brine, dried over anhydrous Na₂SO₄ to get the crude product. The residue was purified by reverse-phase flash chromatography with the following conditions: column, C18 silica gel; mobile phase, MeCN in Water, 0% to 100% gradient in 10 min; detector, UV 254 nm to afford methyl 5,7-difluoro-1,3-dimethylindole-2-carboxylate (3-d; 1.325 g, 80%) yellow solid as product. MS (ESI): mass calcd. for C₁₂H₁₁F₂NO₂, 239.2. m/z found 240.0 [M+H]⁺.

### Step 4

A solution of methyl 5,7-difluoro-1,3-dimethylindole-2-carboxylate (3-d; 1.3 g, 5.43 mmol, 1 equiv) and LiAlH₄ (412.46 mg, 10.87 mmol, 2 equiv) in THF (15 mL) was stirred for 2h from 0° C. to room temperature. After the reaction was completed, the solution was quenched with NH₄C1, extracted with ethyl acetate, washed with brine, dried over anhydrous Na₂SO₄ to provide (5,7-difluoro-1,3-dimethylindol-2-yl) methanol (3-f; 1.1 g, 96%) as a yellow solid.

### Step 5

A solution of (5,7-difluoro-1,3-dimethylindol-2-yl) methanol (3-f; 1.05 g, 4.97 mmol, 1 equiv) and Dess-Martin periodinane (DMP; 3.16 g, 7.46 mmol, 1.5 equiv) in DCM (50 mL) was stirred for 3 h at room temperature. After the reaction was completed, the reaction mixture was quenched with saturated NaHCO₃(aq) and filtrated. The filtrate was extracted with DCM, then dried over anhydrous Na₂SO₄ to provide crude product, which was purified by reserve-phase flash chromatography with the following conditions: column, C18 silica gel; mobile phase, MeCN in Water, 0% to 100% gradient in 10 min; detector, UV 254 nm to afford 5,7-difluoro-1,3-dimethylindole-2-carbaldehyde (3-g; 600 mg, 58%) yellow solid as product. MS (ESI): mass calcd. for C₁₁H₉F₂NO, 209.2, m/z found 210.1 [M+H]⁺.

### Step 6

A solution of 5,7-difluoro-1,3-dimethylindole-2-carbaldehyde (3-g; 607 mg, 2.90 mmol, 1 equiv), K₂CO₃ (802.03 mg, 5.80 mmol, 2 equiv) and TMSCF₃ (825.19 mg, 5.80 mmol, 2 equiv) in DMF (10 mL) was stirred overnight at room temperature. After the reaction was completed, the reaction mixture was quenched by water, extracted with water, washed with the brine, dried over anhydrous Na₂SO₄, and concentrated under reduced pressure to afford 1-(5,7-difluoro-1,3-dimethylindol-2-yl)-2,2,2-trifluoroethanol (3-h; 580 mg, 71.59%) as a light-yellow solid. MS (ESI): mass calcd. for C12H₁₀F₅NO, 279.2, m/z found 280.15 [M+H]⁺.

### Step 7

A solution of 1-(5,7-difluoro-1,3-dimethylindol-2-yl)-2,2,2-trifluoroethanol (3-h; 2.1 g, 7.52 mmol, 1 equiv) and DMP (4.79 g, 11.28 mmol, 1.5 equiv) in DCM (70 mL) was stirred for 4 h at room temperature. After the reaction was completed, the solution was quenched with saturated NaHCO₃(aq) and filtrated. The filtrate was extracted with DCM and dried over anhydrous Na₂SO₄ to obtain crude product, which was purified by reserve-phase flash chromatography to afford 1-(5,7-difluoro-1,3-dimethylindol-2-yl)-2,2,2-trifluoroethanone (3-i; 1.8 g, 86%) as a yellow solid.

### Step 8

A solution of 1-(5,7-difluoro-1,3-dimethylindol-2-yl)-2,2,2-trifluoroethanone (3-i; 831 mg, 3.00 mmol, 1 equiv), NH₂OH·HCl (1041.62 mg, 14.99 mmol, 5 equiv) and NaOAc (1229.65 mg, 14.99 mmol, 5 equiv) in EtOH (30 mL) was stirred overnight at 80° C. After the reaction was completed, the reaction mixture was purified by reserve-phase flash chromatography with the following conditions: column, C18 silica gel; mobile phase, MeCN in Water, 0% to 100% gradient in 10 min; detector, UV 254 nm to afford (Z)-N-[1-(5,7-difluoro-1,3-dimethylindol-2-yl)-2,2,2-trifluoroethylidene]hydroxylamine (3-j; 720 mg, 82%) as a yellow solid. MS (ESI): mass calcd. for C₁₂H₉F₅N₂O, 292.2, m/z found 293.0[M+H]⁺.

### Step 9

A solution of (Z)-N-[1-(5,7-difluoro-1,3-dimethylindol-2-yl)-2,2,2trifluoroethylidene]hydroxylamine (3-j; 643.3 mg, 2.20 mmol, 1 equiv), Zn powder (1439.34 mg, 22.02 mmol, 10 equiv) and NH₄Cl (588.79 mg, 11.01 mmol, 5 equiv) in EtOH (30 mL) and H₂O (10 mL) was stirred overnight at 80° C. After the reaction was completed, the reaction mixture was purified by reserve-phase flash chromatography with the following conditions: column, C18 silica gel; mobile phase, MeCN in Water, 0% to 100% gradient in 10 min; detector, UV 254 nm to obtain 1-(5,7-difluoro-1,3-dimethylindol-2-yl)-2,2,2-trifluoroethanamine (3-k; 252 mg, 41%) yellow solid. MS (ESI): mass calcd. for C₁₂H₁₁F₅N₂, 278.2, m/z found 262.05 [M-NH₃+H]⁺.

### Step 10

A solution of 1-(5,7-difluoro-1,3-dimethylindol-2-yl)-2,2,2-trifluoroethanamine (3-k; 132 mg, 0.47 mmol, 1 equiv) in phenyl N-(2-aminopyrimidin-5-yl) carbamate (3 mL) was stirred overnight at 80° C. After the reaction was completed, the reaction mixture was concentrated under reduced pressure, then purified by reverse-phase flash chromatography to obtain the crude product, which was purified by Prep-HPLC with the following conditions (Column: Xselect CSH C18 OBD Column 30*150 mm 5 m, n; Mobile Phase A: Water(0.1% FA), Mobile Phase B: ACN; Flow rate: 60 mL/min; Gradient: 33% B to 47% B in 8 min, 47% B; Wave Length: 254; 220 nm; RT1(min): 7.65; Injection Volume: 1.5 mL) to to obtain 35 mg of racemic product.

### Step 11

The racemic product mixture from Step 10 was purified by Prep-Chiral-HPLC (Column: (R, R)-WHELK-01-Kromasi, 5*25 cm, 5 m; Mobile Phase A: Hex(0.5% 2M NH₃-MeOH)— HPLC, Mobile Phase B: EtOH— HPLC; Flow rate: 20 mL/min; Gradient: 40% B to 40% B in 13 min; Wave Length: 220/254 nm; RT1(min): 5.45; RT2(min): 11.55; Sample Solvent: EtOH— HPLC; Injection Volume: 2.65 mL; Number Of Runs: 1) to obtain 1-(2-aminopyrimidin-5-yl)-3-[(1R)-1-(5,7-difluoro-1,3-dimethylindol-2-yl)-2,2,2-trifluoroethyl]ureas (Compound 3; 19.8 mg, 10%) as an off-white solid. MS (ESI): mass calcd. for C₁₇H₁₅F₅N₆O, 414.3, m/z found 415.2 [M+H]⁻. ¹H NMR (400 MHz, DMSO) δ 8.35 (s, 1H), 8.22 (s, 1H), 7.63 (d, J=8.8 Hz, 1H), 7.24 (dd, J=2.4, 8.2 Hz, 1H), 7.09-7.03 (m, 1H), 6.39 (s, 2H), 6.06 (d, J=8.8 Hz, 1H), 3.96 (s, 3H), 2.33 (s, 3H).

### Example 4: Preparation of Compound 4 and Compound 5

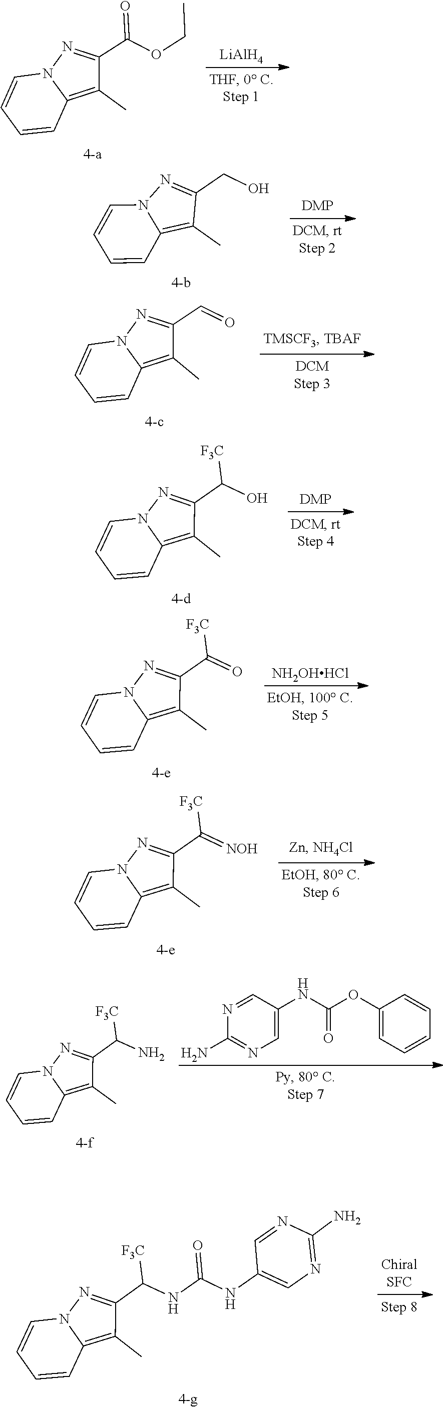

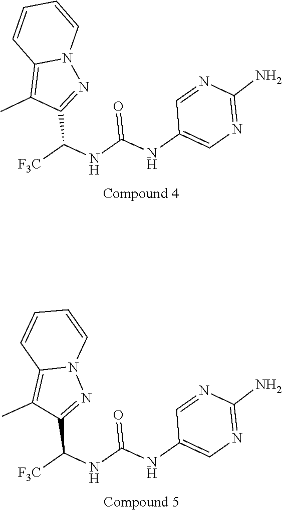 [SMILES: Cc1c([C@H](NC(=O)Nc2cnc(N)nc2)C(F)(F)F)nn2ccccc12 ; Cc1c([C@@H](NC(=O)Nc2cnc(N)nc2)C(F)(F)F)nn2ccccc12]

### Step 1

A solution of ethyl 3-methylpyrazolo[1,5-a]pyridine-2-carboxylate (4-a; 500 mg, 2.448 mmol, 1 equiv) and LiAlH₄ (92.91 mg, 2.448 mmol, 1 equiv) in THF (10 mL, 61.714 mmol) was stirred for 30 min at 0° C. under nitrogen atmosphere. The resulting mixture was stirred for 2 h at room temperature under nitrogen atmosphere. Desired product could be detected by LCMS. The reaction was quenched by the addition of sodium sulfate decahydrate (2 g) at 0° C. The resulting mixture was filtered, the filter cake was washed with EtOAc (3×1 1 mL). The resulting mixture was extracted with EtOAc (2×5 mL). The combined organic layers were washed with brine (4×1 10 mL), dried over anhydrous Na₂SO₄. After filtration, the filtrate was concentrated under reduced pressure. The residue was purified by Prep-TLC (PE/EA 5:1). The resulting mixture was concentrated under reduced pressure to afford {3-methylpyrazolo[1,5-a]pyridin-2-yl}methanol (4-b; 260 mg, 65%) as a light brown solid. MS (ESI): mass calcd. for C₉H₁₀N₂O, 162.1, m/z found 163.3 [M+H]⁺.

### Step 2

A solution of {3-methylpyrazolo[1,5-a]pyridin-2-yl}methanol (4-b; 200 mg, 1.233 mmol, 1 equiv) and DMP (1046.03 mg, 2.466 mmol, 2 equiv) in DCM (10 mL) was stirred for 3 h at room temperature. Desired product could be detected by LCMS. The resulting mixture was filtered, and the filter cake was washed with EtOAc (3×1 mL). The filtrate was concentrated under reduced pressure. The residue was purified by Prep-TLC (PE/EA 5:1) to afford 3-methylpyrazolo[1,5-a]pyridine-2-carbaldehyde (4-c; 160 mg, 81%) as a light yellow solid. MS (ESI): mass calcd. for C₉H₈N₂O, 161.1, m/z found 161.2 [M+H]⁺.

### Step 3

Into a 40 mL vial were added 3-methylpyrazolo[1,5-a]pyridine-2-carbaldehyde (4-c; 400 mg, 2.497 mmol, 1 equiv), trifluoromethyltrimethylsilane (1065.30 mg, 7.491 mmol, 3 equiv) and THF (10 mL) at room temperature. The mixture was cooled to 0° C. and TBAF (652.94 mg, 2.497 mmol, 1 equiv) was added with stirring at 0° C. The resulting mixture was stirred for additional 12 h at room temperature. Desired product could be detected by LCMS. The resulting mixture was extracted with EtOAc (3×5 mL). The combined organic layers were washed with brine (3×1 10 mL), dried over anhydrous Na₂SO₄. After filtration, the filtrate was concentrated under reduced pressure. The residue was purified by Prep-TLC (PE/EA 3:1) to afford 2,2,2-trifluoro-1-{3-methylpyrazolo[1,5-a]pyridin-2-yl}ethanol) (4-d; 444 mg, 77%) as a light brown solid. MS (ESI): mass calcd. for C₁₀H₉F₃N₂O, 230.1, m/z found 231.2 [M+H]⁺.

### Step 4

A solution of 2,2,2-trifluoro-1-{3-methylpyrazolo[1,5-a]pyridin-2-yl}ethanol (4-d; 420 mg, 1.825 mmol, 1 equiv) and DMP (1547.77 mg, 3.650 mmol, 2 equiv) in DCM (10 mL) was stirred for 2 h at room temperature under nitrogen atmosphere. The resulting mixture was concentrated under reduced pressure. The residue was purified by Prep-TLC (PE/EA 1:1) to afford 2,2,2-trifluoro-1-{3-methylpyrazolo[1,5-a]pyridin-2-yl}ethanone (4-e; 416 mg, 100%) as a yellow solid. MS (ESI): mass calcd. for C₁₀H₇F₃N₂O, 228.2, m/z found 247.2 [M+H+H₂O]⁺.

### Step 5

To a stirred solution of 2,2,2-trifluoro-1-{3-methylpyrazolo[1,5-a]pyridin-2-yl}ethanone (4-e; 300 mg, 1.446 mmol, 1 equiv) and NH₂OH·HCl (502.50 mg, 7.230 mmol, 5 equiv) in EtOH (20 mL) was added NaOAc (593.21 mg, 7.23 mmol, 5 equiv) at room temperature under air atmosphere. The resulting mixture was stirred for 2 h at 80° C. under nitrogen atmosphere. Desired product could be detected by LCMS. The resulting mixture was extracted with EtOAc (2×10 mL). The combined organic layers were washed with brine (4×1 20 mL), dried over anhydrous Na₂SO₄. After filtration, the filtrate was concentrated under reduced pressure. The residue was purified by Prep-TLC (PE/EA 5:1) to afford (Z)-N-(2,2,2-trifluoro-1-{3-methylpyrazolo[1,5-a]pyridin-2-yl}ethylidene) hydroxylamine (4-e; 180 mg, 56%) as a white solid. MS (ESI): mass calcd. for C₁₀H₈F₃N₃O, 243.2, m/z found 244.2 [M+H]⁺.

### Step 6

To a stirred solution of (Z)-N-(2,2,2-trifluoro-1-{3-methylpyrazolo[1,5-a]pyridin-2-yl}ethylidene) hydroxylamine (4-e; 150 mg, 0.617 mmol, 1 equiv) and Zn powder (403.27 mg, 6.170 mmol, 10 equiv) in EtOH (3 mL) was added NH₄Cl (329.93 mg, 6.170 mmol, 10 equiv) dropwise at room temperature under nitrogen atmosphere. The resulting mixture was filtered, and the filter cake was washed with EtOAc (4×1 1 mL). The filtrate was concentrated under reduced pressure. The residue was purified by reverse-phase flash chromatography with the following conditions: column, silica gel; mobile phase, MeCN in water, 10% to 50% gradient in 10 min; detector, UV 254 nm. The resulting mixture was concentrated under reduced pressure to afford 2,2,2-trifluoro-1-{3-methylpyrazolo[1,5-a]pyridin-2-yl}ethanamine (4-f; 80 mg, 57%) as a white solid. MS (ESI): mass calcd. for C₁₀H₁₀F₃N₃, 229.2, m/z found 230.2 [M+H]⁺.

### Step 7

A solution of 2,2,2-trifluoro-1-{3-methylpyrazolo[1,5-a]pyridin-2-yl}ethanamine (4-f; 100 mg, 0.436 mmol, 1 equiv) and phenyl N-(2-aminopyrimidin-5-yl)carbamate (100.45 mg, 0.436 mmol, 1 equiv) in Pyridine (3 mL) was stirred for 4 h at 80° C. under nitrogen atmosphere. Desired product could be detected by LCMS. The resulting mixture was extracted with EtOAc (2×5 mL). The combined organic layers were washed with brine (4×1 10 mL), and dried over anhydrous Na₂SO₄. After filtration, the filtrate was concentrated under reduced pressure. The residue was purified by trituration with MeCN (1 mL). The resulting mixture was filtered, the filter cake was washed with MeCN (3×1 1 mL). The filtrate was concentrated under reduced pressure to afford crude product (4-g, 50 mg) as an off-white solid. MS (ESI): mass calcd. for C₁₅H₁₄F₃N₇0, 365.1, m/z found 366.2 [M+H]⁺.

### Step 8

50 mg of racemic 4-g was purified by chiral SFC to Compound 4 (9 mg as white solid) and Compound 5 (5 mg as a white solid).

### Chiral Separation Conditions:

Apparatus: SFC 80
Column: DZ-CHIRALPAK IG-3, 4.6*50 mm, 3.0 m
Mobile Phase A: Hex(0.2% DEA): (EtOH: DCM=1: 1)=70: 30
Flow rate: 1 mL/min
Gradient: 0% B to 0% B
Injection Volume: 5 ul mL

Compound 4: MS (ESI): mass calcd. for C₁₅H₁₄F₃N₇O, 365.1, m/z found 366.2 [M+H]⁺.

¹H NMR (400 MHz, DMSO-d6) δ 8.63 (dt, J=7.0, 1.1 Hz, 1H), 8.43 (s, 1H), 8.22 (s, 2H), 7.70 (dt, J=8.9, 1.2 Hz, 1H), 7.40 (d, J=9.2 Hz, 1H), 7.24 (ddd, J=9.0, 6.7, 1.0 Hz, 1H), 6.94 (td, J=6.9, 1.4 Hz, 1H), 6.37 (s, 2H), 5.85 (p, J=8.3 Hz, 1H), 2.30 (s, 3H).

Compound 5: MS (ESI): mass calcd. for C₁₅H₁₄F₃N₇O, 365.1, m/z found 366.2 [M+H]⁺.

¹H NMR (400 MHz, DMSO-d6) δ 8.63 (d, J=7.0 Hz, 1H), 8.43 (s, 1H), 8.22 (s, 2H), 7.70 (dd, J=9.0, 1.3 Hz, 1H), 7.40 (d, J=9.2 Hz, 1H), 7.24 (dd, J=9.0, 6.7 Hz, 1H), 6.94 (td, J=6.8, 1.4 Hz, 1H), 6.37 (s, 2H), 5.85 (p, J=8.2 Hz, 1H), 2.30 (s, 3H).

### Example 5: Preparation of Compound 6 and Compound 7

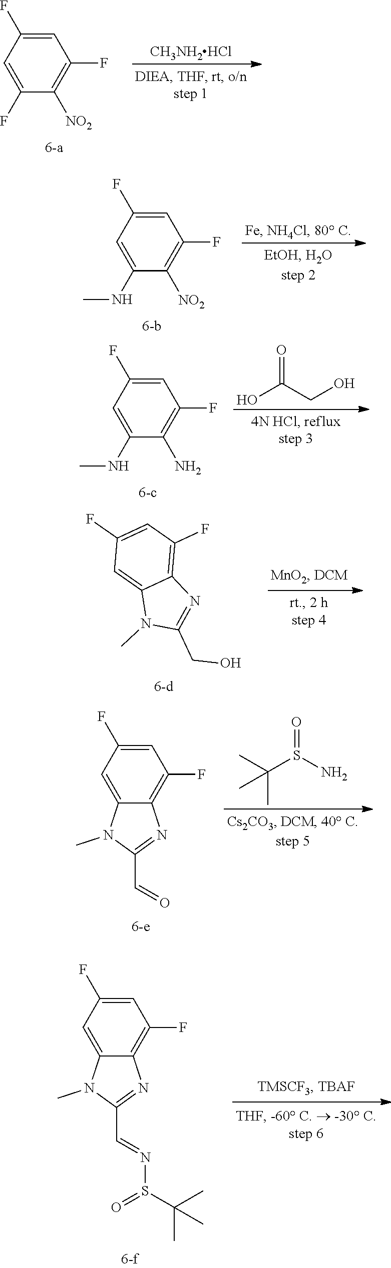

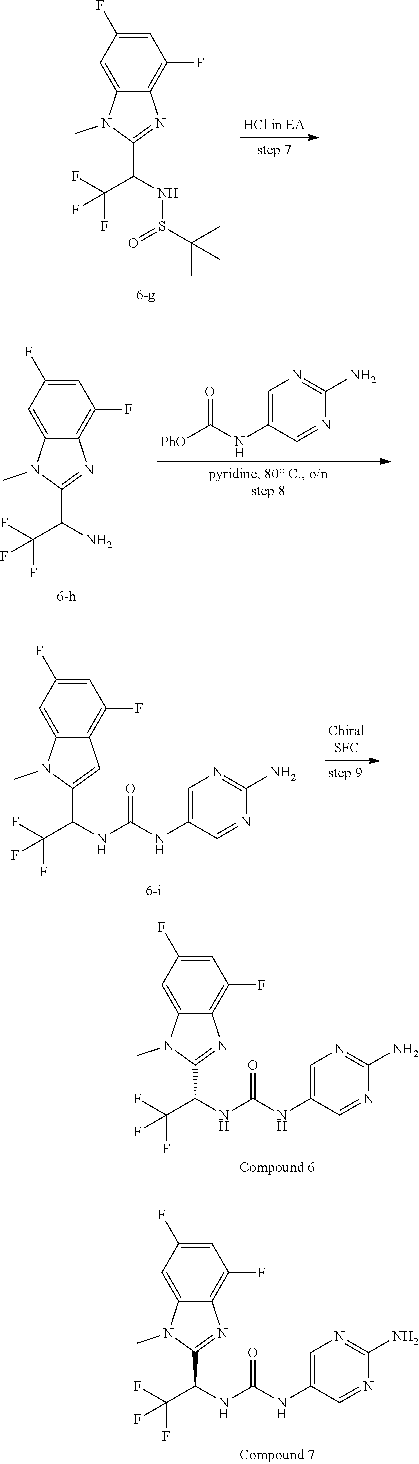 [SMILES: Cn1c(C(NS(=O)C(C)(C)C)C(F)(F)F)nc2c(F)cc(F)cc21 ; Cn1c(C(N)C(F)(F)F)nc2c(F)cc(F)cc21 ; Nc1ncc(NC(=O)Oc2ccccc2)cn1 ; Cn1c(C(NC(=O)Nc2cnc(N)nc2)C(F)(F)F)cc2c(F)cc(F)cc21 ; Cn1c([C@H](NC(=O)Nc2cnc(N)nc2)C(F)(F)F)nc2c(F)cc(F)cc21 ; Cn1c([C@@H](NC(=O)Nc2cnc(N)nc2)C(F)(F)F)nc2c(F)cc(F)cc21]

### Step 1

To a stirred solution of 1,3,5-trifluoro-2-nitrobenzene (6-a; 1.00 g, 5.65 mmol, 1.00 equiv) and DIEA (2.19 g, 16.94 mmol, 3.00 equiv) in THF (10 mL) was added methanamine hydrochloride (570 mg, 8.47 mmol, 1.50 equiv) in portions at 0° C. under nitrogen atmosphere. The resulting mixture was stirred overnight at room temperature under nitrogen atmosphere. The resulting mixture was concentrated under reduced pressure. The residue was purified by silica gel column chromatography, eluted with PE/EA (10:1) to afford 3,5-difluoro-N-methyl-2-nitroaniline (6-b; 1.05 g, 99%) as a yellow solid. MS (ESI): mass calcd. for C₇H₆F₂N₂O₂, 188.04, m/z found 189.00 [M+H]⁺.

### Step 2

A mixture of 3,5-difluoro-N-methyl-2-nitroaniline (6-b; 1.05 g, 5.59 mmol, 1.00 equiv) and Fe powder (1.56 g, 27.95 mmol, 5.00 equiv), NH₄Cl(1.50 g, 27.95 mmol, 5.00 equiv) in EtOH (10 mL) and H₂O (2 mL) was stirred for 1 h at 80° C. under nitrogen atmosphere. The resulting mixture was filtered, the filter cake was washed with MeOH (3×20 mL). The filtrate was concentrated under reduced pressure. The residue was purified by silica gel column chromatography, eluted with PE/EA (5:1) to afford 3,5-difluoro-N1-methylbenzene-1,2-diamine (6-c; 890 mg, 88%) as a brown oil. MS (ESI): mass calcd. for C₇H₈F₂N₂, 158.07, m/z found 159.05 [M+H]⁺.

### Step 3

To an aqueous solution of HCl (10 mL) (4M) and glycolic acid (481 mg, 6.32 mmol, 1.00 equiv) was added 3,5-difluoro-N1-methylbenzene-1,2-diamine (6-c; 1.00 g, 6.32 mmol, 1.00 equiv) and the reaction mixture was refluxed overnight. The reaction mixture was cooled to 0° C. and then alkalized with a ˜40% NaOH aqueous solution up to pH=8. The precipitated solids were collected by filtration and washed with water (3×30 mL) to afford (4,6-difluoro-1-methyl-1,3-benzodiazol-2-yl)methanol (6-d; 1 g, 80%) as a grey solid. MS (ESI): mass calcd. for C₉H₈F₂N₂O, 198.06, m/z found 199.00 [M+H]⁺.

### Step 4

A mixture of (4,6-difluoro-1-methyl-1,3-benzodiazol-2-yl)methanol (6-d; 1.00 g, 5.05 mmol, 1.00 equiv) and MnO₂ (4.39 g, 50.46 mmol, 10.00 equiv) in DCM (15 mL) was stirred overnight at room temperature under nitrogen atmosphere. The resulting mixture was filtered, and the filter cake was washed with ethyl acetate (3×30 mL). The filtrate was concentrated under reduced pressure. The residue was purified by silica gel column chromatography, eluted with EA to afford 4,6-difluoro-1-methyl-1,3-benzodiazole-2-carbaldehyde (6-e; 480 mg, 48%) as a yellow solid. MS (ESI): mass calcd. for C₉H₆F₂N₂O, 196.04, m/z found 197.00 [M+H]⁺.

### Step 5

To a solution of 4,6-difluoro-1-methyl-1,3-benzodiazole-2-carbaldehyde (6-e; 200 mg, 1.02 mmol, 1.00 equiv) in DCM (2 mL) was added Cs₂CO₃ (339 mg, 1.04 mmol, 1.02 equiv) at 25° C. And the reaction mixture was stirred at 25° C. for 10 min, then tert-butanesulfinamide (140 mg, 1.15 mmol, 1.13 equiv) was added. The reaction mixture was stirred at 40° C. overnight. The reaction mixture was filtered and concentrated under reduced pressure to give a residue, which was purified by silica gel column chromatography eluted with PE/EA (3:1) to afford N-[(1LE)-(4,6-difluoro-1-methyl-1,3-benzodiazol-2-yl)methylidene]-2-methylpropane-2-sulfinamide (6-f; 250 mg, 82%) as a light yellow solid. MS (ESI): mass calcd. for C₁₃H₁₅F₂N₃OS, 299.09, m/z found 300.00 [M+H]⁺.

### Step 6

To a solution of N-[(1E)-(4,6-difluoro-1-methyl-1,3-benzodiazol-2-yl)methylidene]-2-methylpropane-2-sulfinamide (6-f; 200 mg, 0.67 mmol, 1.00 equiv) in THF (5 mL) at 0° C. was added difluorotriphenylsilanuide; tetrabutylazanium (361 mg, 0.67 mmol, 1.00 equiv) and TMSCF₃ (380 mg, 2.67 mmol, 4.000 equiv) was added at 0° C. The mixture was stirred at 0° C. for 1 h. The reaction was quenched with sat. NH₄Cl (aq.) at 0° C. then extracted with EtOAc (2×30 mL). The combined organic layers were dried over anhydrous Na₂SO₄. After filtration, the filtrate was concentrated under reduced pressure. The crude N-[1-(4,6-difluoro-1-methyl-1,3-benzodiazol-2-yl)-2,2,2-trifluoroethyl]-2-methylpropane-2-sulfinamide (6-g; 300 mg) as a yellow oil was used in the next step directly without further purification. MS (ESI): mass calcd. for C14H₁₆F₅N₃OS, 369.09, m/z found 370.10 [M+H]⁺.

### Step 7

To a solution of N-[1-(4,6-difluoro-1-methyl-1,3-benzodiazol-2-yl)-2,2,2-trifluoroethyl]-2-methylpropane-2-sulfinamide (6-g; 300 mg, 0.81 mmol, 1.00 equiv) in EA (4 mL) was added HCl (2 mL) (4 M in EA) at 0° C. The mixture was stirred at room temperature for 1 hr. The resulting mixture was concentrated and purified by silica gel column chromatography, eluted with PE/EA (1:1) to afford 1-(4,6-difluoro-1-methyl-1,3-benzodiazol-2-yl)-2,2,2-trifluoroethanamine (6-h; 60 mg, 28%) as a light yellow oil. MS (ESI): mass calcd. for C₁₀H₈F₅N₃, 265.06, m/z found 266.05 [M+H]⁺.

### Step 8

A solution of 1-(4,6-difluoro-1-methyl-1,3-benzodiazol-2-yl)-2,2,2-trifluoroethanamine (6-h; 60 mg, 0.23 mmol, 1.00 equiv) and phenyl N-(2-aminopyrimidin-5-yl)carbamate (53 mg, 0.23 mmol, 1.00 equiv) in pyridine (1.5 mL) was stirred overnight at 80° C. under nitrogen atmosphere. The resulting mixture was concentrated under reduced pressure. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm to afford 1-(2-aminopyrimidin-5-yl)-3-[1-(4,6-difluoro-1-methyl-1,3-benzodiazol-2-yl)-2,2,2-trifluoroethyl]urea (6-i; 60 mg, 66%) as a light yellow solid. MS (ESI): mass calcd. for C₁₅H₁₂F₅N₇O, 401.10, m/z found 402.10 [M+H]⁺.

### Step 9

60 mg of racemic 6-i was separated by CHIRAL-HPLC to give Compound 6 (22.8 mg as white solid) and Compound 7 (18.0 mg as white solid).

### Chiral Separation Condition:

Apparatus: Prep-HPLC-037
Column: CHIRALPAK IG, 2*25 cm, 5 m
Mobile phase: Mobile Phase A: Hex(0.5% 2M NH₃-MeOH)— HPLC, Mobile Phase B: EtOH:
DCM=1: 1— HPLC
Flow rate: 20 mL/min
Wavelength: UV 220/254 nm
Temperature: 25° C.

Compound 6: MS (ESI): mass calcd. for C₁₅H₁₂F₅N₇O, 401.10, m/z found 402.10 [M+H]+.

¹H NMR (400 MHz, DMSO-d₆) δ 8.41 (s, 1H), 8.22 (s, 2H), 7.71 (d, J=9.1 Hz, 1H), 7.50 (dd, J=8.9, 2.3 Hz, 1H), 7.18 (td, J=10.6, 2.3 Hz, 1H), 6.39 (s, 2H), 6.27-6.13 (m, 1H), 3.89 (s, 3H).

Compound 7: MS (ESI): mass calcd. for C₁₅H₁₂F₅N₇0, 401.10, m/z found 402.10 [M+H]+.

¹H NMR (400 MHz, DMSO-d₆) δ 8.42 (s, 1H), 8.22 (s, 2H), 7.71 (d, J=9.1 Hz, 1H), 7.50 (dd, J=9.0, 2.2 Hz, 1H), 7.18 (td, J=10.6, 2.2 Hz, 1H), 6.40 (s, 2H), 6.21 (p, J=7.2 Hz, 1H), 3.89 (s, 3H).

### Example 6: Preparation of Compound 8 and Compound 9

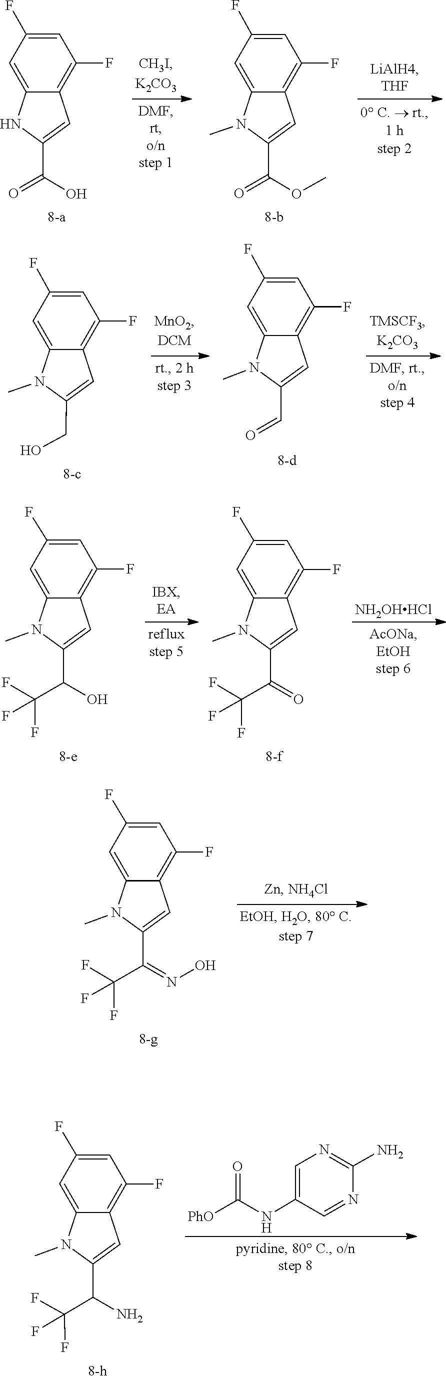

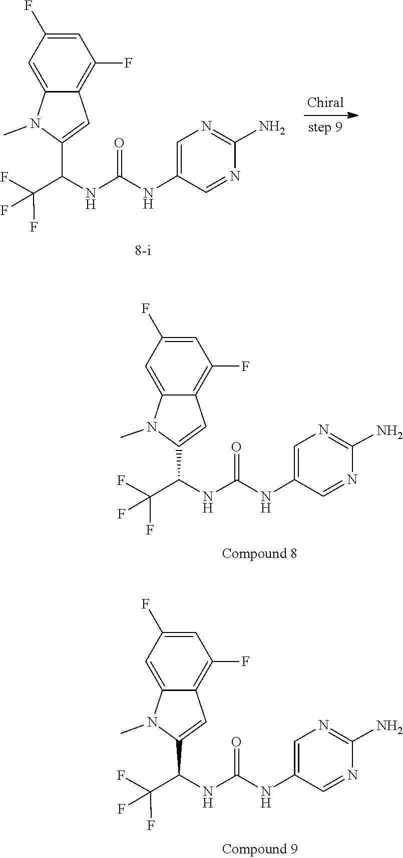 [SMILES: Cn1c(C(NC(=O)Nc2cnc(N)nc2)C(F)(F)F)cc2c(F)cc(F)cc21 ; Cn1c([C@H](NC(=O)Nc2cnc(N)nc2)C(F)(F)F)cc2c(F)cc(F)cc21 ; Cn1c([C@@H](NC(=O)Nc2cnc(N)nc2)C(F)(F)F)cc2c(F)cc(F)cc21]

### Step 1

To a stirred solution of 4,6-difluoro-1H-indole-2-carboxylic acid (8-a; 2.00 g, 10.15 mmol, 1.00 equiv) and K₂CO₃ (4.21 g, 30.44 mmol, 3.00 equiv) in DMF (20 mL) was added CH₃I (2.53 mL, 40.58 mmol, 4.00 equiv) dropwise at 0° C. under nitrogen atmosphere. The solution was stirred overnight at room temperature under nitrogen atmosphere. The reaction was monitored by LCMS. The reaction was quenched with sat. NH₄Cl (aq.) at 0° C. The precipitated solids were collected by filtration and washed with water (1×50 mL). The resulted methyl 4,6-difluoro-1-methylindole-2-carboxylate (8-b; 2.1 g, 92%) as a white solid. MS (ESI): mass calcd. for C₁₁H₉F₂NO₂, 225.06, m/z found 226.15 [M+H]⁺.

### Step 2

To a stirred solution of methyl 4,6-difluoro-1-methylindole-2-carboxylate (8-b; 1.00 g, 4.44 mmol, 1.00 equiv) in THF (5 mL) was added LiAlH₄ (solution in THF; 5.33 mL, 5.33 mmol, 1.20 equiv) dropwise at 0° C. under nitrogen atmosphere. The solution was stirred for 1 h at room temperature under nitrogen atmosphere. The reaction was monitored by LCMS. The reaction was quenched with sodium sulfate decahydrate at 0° C. The resulting mixture was filtered, the filter cake was washed with ethyl acetate (1×100 mL). The filtrate was concentrated under reduced pressure to afford (4,6-difluoro-1-methylindol-2-yl)methanol (8-c; 895 mg) as a white solid. The crude product was used in the next step directly without further purification. MS (ESI): mass calcd. for C₁₀H₉F₂NO, 197.07, m/z found 198.15 [M+H]⁺.

### Step 3

To a stirred solution of (4,6-difluoro-1-methylindol-2-yl)methanol (8-c; 850 mg, 4.31 mmol, 1.00 equiv) in DCM (10 mL) was added MnO₂ (7.50 g, 86.22 mmol, 20.00 equiv) in portions at 0° C. under nitrogen atmosphere. The solution was stirred for 1 days at room temperature under nitrogen atmosphere. The reaction was monitored by LCMS. The resulting mixture was filtered, the filter cake was washed with ethyl acetate (1×100 mL). The filtrate was concentrated under reduced pressure to afford 4,6-difluoro-1-methylindole-2-carbaldehyde (8-d; 880 mg) as a white solid. The crude product was used in the next step directly without further purification. MS (ESI): mass calcd. for C₁₀H₇F₂NO, 195.05, m/z found 196.00 [M+H]⁺.

### Step 4

To a stirred solution of 4,6-difluoro-1-methylindole-2-carbaldehyde (8-d; 895 mg, 4.59 mmol, 1.00 equiv) and K₂CO₃ (1.90 g, 13.76 mmol, 3.00 equiv) in DMF (10 mL) was added TMSCF₃ (1.30 g, 9.17 mmol, 2.00 equiv) dropwise at 0° C. under nitrogen atmosphere. The solution was stirred for 2 h at room temperature under nitrogen atmosphere. The reaction was monitored by LCMS. The reaction was quenched with TBAF at 0° C. The resulting mixture was diluted with water (200 mL). The resulting mixture was extracted with EtOAc (3×100 mL). The combined organic layers were washed with brine (1×200 mL), dried over anhydrous Na₂SO₄. After filtration, the filtrate was concentrated under reduced pressure to afford 1-(4,6-difluoro-1-methylindol-2-yl)-2,2,2-trifluoroethanol (8-e; 1.02 g, 84%) as a yellow oil. MS (ESI): mass calcd. for C₁₁H₈F₅NO, 265.05, m/z found 266.00 [M+H]⁺.

### Step 5

To a stirred solution of 1-(4,6-difluoro-1-methylindol-2-yl)-2,2,2-trifluoroethanol (8-e; 1.02 g, 3.85 mmol, 1.00 equiv) in EA (10 mL) was added IBX (2.15 g, 7.69 mmol, 2.00 equiv) in portions at 0° C. under nitrogen atmosphere. The solution was stirred overnight at 80° C. under nitrogen atmosphere. The reaction was monitored by TLC. The resulting mixture was filtered, the filter cake was washed with ethyl acetate (1×100 mL). The filtrate was concentrated under reduced pressure. The residue was purified by silica gel column chromatography, eluted with PE to afford 1-(4,6-difluoro-1-methylindol-2-yl)-2,2,2-trifluoroethanone (8-f; 910 mg, 90%) as a white solid.

### Step 6

To a stirred solution of 1-(4,6-difluoro-1-methylindol-2-yl)-2,2,2-trifluoroethanone (8-f; 850 mg, 3.23 mmol, 1.00 equiv) and AcONa (1.32 g, 16.15 mmol, 5.00 equiv) in EtOH (8 mL) was added NH₂OH·HCl (1.12 g, 16.15 mmol, 5.00 equiv) in portions at 0° C. under nitrogen atmosphere. The solution was stirred overnight at 80° C. under nitrogen atmosphere. The reaction was monitored by LCMS. The resulting mixture was diluted with water (100 mL). The resulting mixture was extracted with EtOAc (3×100 mL). The combined organic layers were washed with brine (1×200 mL), dried over anhydrous Na₂SO₄. After filtration, the filtrate was concentrated under reduced pressure. The residue was purified by Prep-TLC (PE/EA 5:1) to afford (E)-N-[1-(4,6-difluoro-1-methylindol-2-yl)-2,2,2-trifluoroethylidene]hydroxylamine (8-g; 890 mg, 99%) as a yellow solid. MS (ESI): mass calcd. for C₁₁H₇F₅N₂O, 278.05, m/z found 277.00[M−H]⁻.

### Step 7

To a stirred solution of (E)-N-[1-(4,6-difluoro-1-methylindol-2-yl)-2,2,2-trifluoroethylidene]hydroxylamine (8-g; 850 mg, 3.06 mmol, 1.00 equiv) and NH₄Cl(1.63 g, 30.56 mmol, 10.00 equiv) in EtOH (7 mL) and H₂O (1.4 mL) was added Zn powder (1.99 g, 30.56 mmol, 10.00 equiv) in portions at 0° C. under nitrogen atmosphere. The solution was stirred for 2 h at 80° C. under nitrogen atmosphere. The reaction was monitored by LCMS. The resulting mixture was filtered, the filter cake was washed with ethyl acetate (1×100 mL). The filtrate was concentrated under reduced pressure. The residue was purified by silica gel column chromatography, eluted with EA to afford 1-(4,6-difluoro-1-methylindol-2-yl)-2,2,2-trifluoroethanamine (8-h; 610 mg, 75.56%) as a brown solid. MS (ESI): mass calcd. for C₁₁H₉F₅N₂, 264.07, m/z found 248.00 [M-NH₄+H]⁺.

### Step 8

To a stirred solution of 1-(4,6-difluoro-1-methylindol-2-yl)-2,2,2-trifluoroethanamine (8-h; 400 mg, 1.51 mmol, 1.00 equiv) in pyridine (4 mL) was added phenyl N-(2-aminopyrimidin-5-yl)carbamate (523 mg, 2.27 mmol, 1.50 equiv) in portions at 0° C. under nitrogen atmosphere. The solution was stirred for 1 day at 80° C. under nitrogen atmosphere. The reaction was monitored by LCMS. The residue was purified by reverse phase flash with the following conditions (column, C18 silica gel; mobile phase, MeCN in Water (0.1% FA), 10% to 50% gradient in 20 min; detector, UV 254 nm) to afford 1-(2-aminopyrimidin-5-yl)-3-[1-(4,6-difluoro-1-methylindol-2-yl)-2,2,2-trifluoroethyl]urea (8-i; 350 mg, 58%) as a white solid. MS (ESI): mass calcd. for C16H₁₃F₅N₆O, 400.11, m/z found 401.05 [M+H]⁺.

### Step 9

Racemic 8-i (350 mg) was separated by CHIRAL-HPLC to give Compound 8 (114.4 mg as white solid) and Compound 9 (105.7 mg as white solid).
Column: CHIRALPAK IG, 2*25 cm, 5 m;
Mobile Phase A: Hex(0.2% TEA)— HPLC, Mobile Phase B: EtOH: DCM=1: 1— HPLC;
Flow rate: 20 mL/min;
Gradient: 30% B to 30% B in 11 min;
Wave Length: 220/254 nm;
RT1(min): 5.64; RT2(min): 8.04;
Sample Solvent: EtOH: DCM=1: 1— HPLC;
Injection Volume: 0.65 mL;
Number Of Runs: 20

Compound 8: MS (ESI): mass calcd. for C₁₆H₁₃F₅N₆O, 400.11, m/z found 401.05 [M+H]⁺.

1H NMR (400 MHz, DMSO-d6) δ 8.22 (s, 2H), 8.07 (s, 1H), 7.62 (d, J=9.3 Hz, 1H), 7.36-7.31 (m, 1H), 6.93-6.92 (m, 1H), 6.69 (s, 1H), 6.40 (s, 2H), 6.02 (p, J=7.9 Hz, 1H), 3.76 (s, 3H).

Compound 9: MS (ESI): mass calcd. for C₁₆H₁₃F₅N₆0, 400.11, m/z found 401.10 [M+H]⁺.

1H NMR (400 MHz, DMSO-d6) δ 8.22 (s, 2H), 8.07 (s, 1H), 7.62 (d, J=9.3 Hz, 1H), 7.36-7.31 (m, 1H), 6.93-6.92 (m, 1H), 6.69 (s, 1H), 6.40 (s, 2H), 6.02 (p, J=8.0 Hz, 1H), 3.76 (s, 3H).

### Example 7: Preparation of Compound 10 and Compound 11

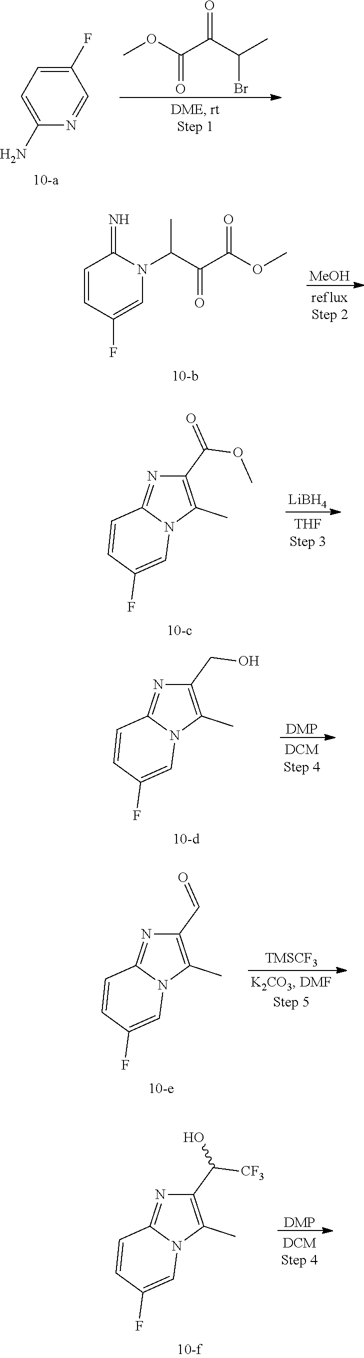

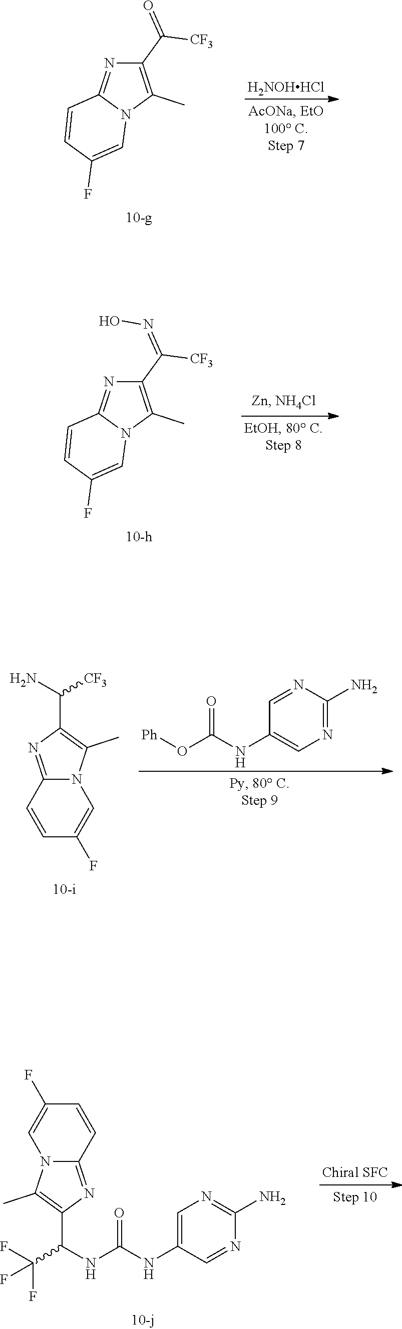

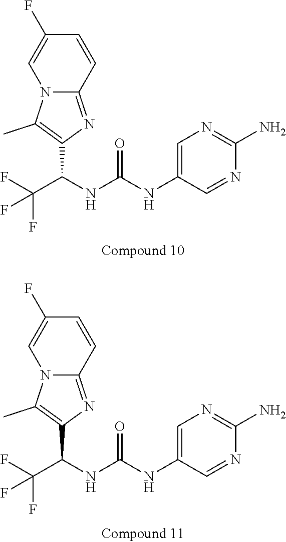 [SMILES: Cc1c([C@H](NC(=O)Nc2cnc(N)nc2)C(F)(F)F)nc2ccc(F)cn12 ; Cc1c([C@@H](NC(=O)Nc2cnc(N)nc2)C(F)(F)F)nc2ccc(F)cn12]

### Step 1

A solution 5-fluoropyridin-2-amine (10-a; 10 g, 89.200 mmol, 1 equiv) and methyl 3-bromo-2-oxobutanoate (16.70 g, 85.632 mmol, 0.96 equiv) in DME was stirred overnight at room temperature under air atmosphere. The reaction was monitored by LCMS. The precipitated solids were collected by filtration and washed with DME (3×5 mL). This resulted in methyl 3-(5-fluoro-2-iminopyridin-1-yl)-2-oxobutanoate (10-b; 19 g, 94%) as an off-white solid. MS (ESI): mass calcd. for C₁₀H₁₁FN₂O₃, 226.1, m/z found 227.1 [M+H]⁺.

### Step 2

A solution of methyl 3-(5-fluoro-2-iminopyridin-1-yl)-2-oxobutanoate (10-b; 19 g) in MeOH (20 mL) was stirred for 3 h at 75° C. Desired product could be detected by LCMS. The resulting mixture was concentrated under reduced pressure. The crude product (10-c) was used in the next step directly without further purification. MS (ESI): mass calcd. for C₁₀H₉FN₂O₂, 208.1, m/z found 209.1 [M+H]⁺.

### Step 3

To a stirred mixture of methyl 6-fluoro-3-methylimidazo[1,2-a]pyridine-2-carboxylate (10-c; 16.4 g, 78.773 mmol, 1 equiv) in THF was added LiBH₄ (59.1 mL, 118.162 mmol, 1.5 equiv) at 0° C. under nitrogen atmosphere. The resulting mixture was stirred overnight at room temperature under nitrogen atmosphere. The reaction was monitored by LCMS. The resulting mixture was concentrated under reduced pressure. This resulted in {6-fluoro-3-methylimidazo[1,2-a]pyridin-2-yl}methanol (10-d; 13 g). The crude product was used in the next step directly without further purification. MS (ESI): mass calcd. for C₉H₉FN₂O, 180.1, m/z found 181.1 [M+H]⁺.

### Step 4

A solution of 2,2,2-trifluoro-1-{6-fluoro-3-methylimidazo[1,2-a]pyridin-2-yl}ethanol (10-d; 3 g, 12.086 mmol, 1 equiv) and DMP (7.687 g, 18.134 mmol, 1.5 equiv) in DCM was stirred for 2 h at room temperature under air atmosphere. The reaction was monitored by LCMS. The resulting mixture was filtered, the filter cake was washed with MeCN (3×100 mL). The filtrate was concentrated under reduced pressure to obtain 2,2,2-trifluoro-1-{6-fluoro-3-methylimidazo[1,2-a]pyridin-2-yl}ethanone (10-e; 1.8 g) as a brown solid that was used crude for the next step. MS (ESI): mass calcd. for C₉H₇FN₂O, 178.1, m/z found 179.1 [M+H]⁺.

### Step 5

A solution of 6-fluoro-3-methylimidazo[1,2-a]pyridine-2-carbaldehyde (10-e; 1.13 g, 6.342 mmol, 1 equiv) in DMF was treated with K₂CO₃ (876.55 mg, 6.342 mmol, 1 equiv) at 0° C. under nitrogen atmosphere followed by the addition of TMSCF₃ (1.80 g, 12.684 mmol, 2 equiv) in portions. The reaction was monitored by LCMS. The resulting mixture was concentrated under reduced pressure. The residue was purified by reverse flash chromatography with the following conditions: column, silica gel; mobile phase, MeCN in water, 0% to 100% gradient in 30 min; detector, UV 254 nm. This resulted in 2,2,2-trifluoro-1-{6-fluoro-3-methylimidazo[1,2-a]pyridin-2-yl}ethanol (10-f; 720 mg, 46%) as a brown solid. MS (ESI): mass calcd. for C₁₀H₈F₄N₂O, 248.1, m/z found 249.1 [M+H]⁺.

### Step 6

A solution of 2,2,2-trifluoro-1-{6-fluoro-3-methylimidazo[1,2-a]pyridin-2-yl}ethanol (10-f; 250 mg, 1.007 mmol, 1 equiv) and DMP (854.50 mg, 2.014 mmol, 2 equiv) in DCM was stirred for 2 h at room temperature under air atmosphere. The reaction was monitored by LCMS. The resulting mixture was filtered, the filter cake was washed with MeCN (1×50 mL). The filtrate was concentrated under reduced pressure. This resulted in 2,2,2-trifluoro-1-{6-fluoro-3-methylimidazo[1,2-a]pyridin-2-yl}ethanone (10-g; 250 mg, 100%) as a brown solid. MS (ESI): mass calcd. for C₁₀H₆F₄N₂O, 246.0, m/z found 247.0 [M+H]⁺.

### Step 7

To a stirred mixture of (3-chlorophenyl)[1-(trifluoromethyl)cyclopropyl]methanone (10-g; 650 mg, 2.614 mmol, 1 equiv) and hydroxylamine hydrochloride (908.35 mg, 13.070 mmol, 5 equiv)in EtOH was added AcONa (1072.32 mg, 13.070 mmol, 5 equiv) in portions at 100° C. under nitrogen atmosphere. The resulting mixture was stirred for 4 h at 100° C. under nitrogen atmosphere. The reaction was monitored by LCMS. The resulting mixture was concentrated under reduced pressure. This resulted in (E)-N-(2,2,2-trifluoro-1-{6-fluoro-3-methylimidazo[1,2-a]pyridin-2-yl}ethylidene)hydroxylamine (10-h; 150 mg, 100.98%) as a brown solid. MS (ESI): mass calcd. for C₁₀H₇F₄N₃0, 261.1, m/z found 262.1 [M+H]⁺.

### Step 8

A solution of (E)-N-(2,2,2-trifluoro-1-{6-fluoro-3-methylimidazo[1,2-a]pyridin-2-yl}ethylidene)hydroxylamine (10-h; 500 mg, 1.91 mmol, 1 equiv) and NH₄Cl (1024.01 mg, 19.140 mmol, 10 equiv) and Zn (1251.63 mg, 19.14 mmol, 10 equiv) in EtOH was stirred overnight at 80° C. under air atmosphere. The reaction was monitored by LCMS. The resulting mixture was filtered, the filter cake was washed with MeCN (3×5 mL). The filtrate was concentrated under reduced pressure. The residue was purified by reverse flash chromatography with the following conditions: column, silica gel; mobile phase, MeCN in water, 0% to 100% gradient in 30 min; detector, UV 254 nm. This resulted in 2,2,2-trifluoro-1-{6-fluoro-3-methylimidazo[1,2-a]pyridin-2-yl}ethanamine (10-i; 200 mg, 42%) as a brown oil. MS (ESI): mass calcd. for C₁₀H₉F₄N₃, 247.1, m/z found 248.1 [M+H]⁺.

### Step 9

A solution of 2,2,2-trifluoro-1-{6-fluoro-3-methylimidazo[1,2-a]pyridin-2-yl}ethanamine (10-i; 150 mg, 0.607 mmol, 1 equiv) and phenyl N-(2-aminopyrimidin-5-yl)carbamate (139.70 mg, 0.607 mmol, 1 equiv) in pyridine was stirred overnight at 80° C. under air atmosphere. The reaction was monitored by LCMS. The resulting mixture was concentrated under vacuum. The residue was purified by Prep-TLC (CH₂Cl₂/MeOH 15:1) to afford 1-(2-aminopyrimidin-5-yl)-3-(2,2,2-trifluoro-1-{6-fluoro-3-methylimidazo[1,2-a]pyridin-2-yl}ethyl)urea (10-j; 15 mg, 6.45%) as an off-white solid. MS (ESI): mass calcd. for C₁₅H₁₃F₄N₇0, 383.1, m/z found 384.1 [M+H]⁺.

### Step 10

Racemic 10-j (20 mg) was purified by Chiral SFC to give Compound 10 (5.5 mg as off-white solid) and Compound 11 (5.5 mg as white solid).
Chiral separation condition:
Column: DZ-CHIRALPAK IG-3, 4.6*50 mm, 3.0 m
Mobile Phase A: Hex(0.2% DEA): EtOH=50: 50
Flow rate: 1 mL/min
Injection Volume: 5 ul mL

Compound 10: MS (ESI):mass calcd. for C₁₅H₁₃F₄N₇0, 383.1, m/z found 384.1 [M+H]⁺.

¹H NMR (400 MHz, Chloroform-d) δ 8.28 (s, 2H), 7.86 (s, 2H), 7.52 (s, 1H), 7.20 (s, 2H), 5.89 (s, 2H), 5.05 (s, 1H), 2.54 (s, 3H).

Compound 11: MS (ESI):mass calcd. for C₁₅H₁₃F₄N₇0, 383.1, m/z found 384.1 [M+H]⁺. ¹H NMR (400 MHz, Chloroform-d) δ 8.28 (s, 2H), 7.85 (s, 2H), 7.50 (dd, J=9.4, 4.9 Hz, 1H), 7.20 (d, J=9.7 Hz, 2H), 5.87 (q, J=7.8 Hz, 2H), 5.04 (s, 1H), 2.53 (s, 3H).

### Assays

### ADP-Glo

Compounds were plated using acoustic liquid dispensing on the Echo 655T by transferring 50 nL/well compound DMSO solution from an Echo source plate to a 384-well assay plate to generate a 10-point dose response curve with a top final concentration of 10 M, 1:3 serial dilution down; the same volume of DMSO was dispensed to wells designated as positive and negative controls. On the day of the assay, a 2X enzyme solution in the assay buffer (50 mM Tris, 150 mM NaCl, 0.01% Brij35, 15 mM MgCl2, 0.05% Tween-20, and 1 mM DTT) was prepared. The enzyme concentrations used were: 20 nM for wild-type, 2 nM for H1047R and H1047L, and 10 nM for H1047Y proteins. 2.5 L/well of 2X enzyme solution was dispensed into the assay plates and the enzyme was incubated with the test compound at room temperature for 1 hour; the same volume of assay buffer without enzyme was dispensed to wells designated as positive controls. At the end of the incubation, 2.5 L/well of 2X ATP solution in assay buffer was dispensed to initiate the ATPase reaction and incubated at room temperature for 100 min. Reactions were terminated and ADP production measured using the ADP-Glo kit following instructions from the manufacturer. See Table 3.

The biological activity of certain compounds using the assays described above is shown in Table 3. The IC₅₀(nM) ranges are as follows: A denotes <200 nM; B denotes 200 nM≤IC₅₀<500 nM; C denotes ≥500 nM. ND denotes value not determined with that assay for the specified compound.

TABLE 3
ADP Glo Data

| | ADP Glo |
|---|---|
| Compound | H1047R |
| No. | IC₅₀ (nM) |
| 1 | A |
| 2 | A |
| 3 | B |
| 4 | ND |
| 5 | C |
| 6 | ND |
| 7 | A |
| 8 | ND |
| 9 | A |
| 10 | ND |
| 11 | C |

1. A compound of Formula (I): 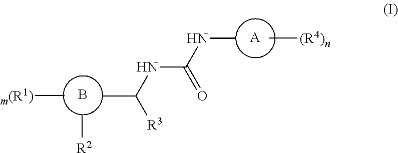
or a pharmaceutically acceptable salt thereof, wherein: 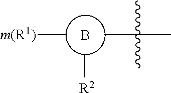
is 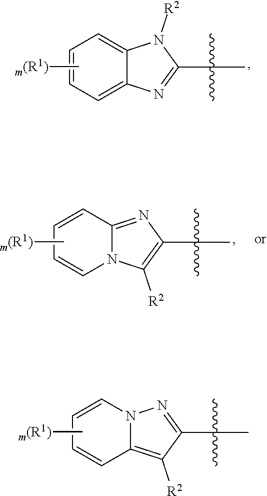
each R¹ is independently selected from halogen;
m is 0, 1, 2, or 3;
R² is an unsubstituted C1-C6 alkyl;
R³ is a C1-C6 alkyl, a C1-C6 haloalkyl;
Ring A is a pyrimidinyl;
R⁴ is —NR^{A}R^{B};
n is 1; and
R^{A} and R^{B} are each hydrogen.

2. (canceled)

3. (canceled)

4. The compound of claim 1, wherein 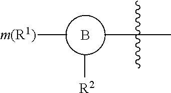
is

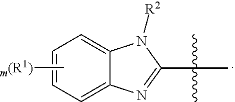

5. The compound of claim 1, wherein 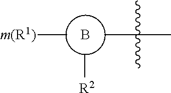
is

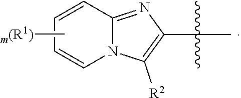

6. The compound of claim 1, wherein 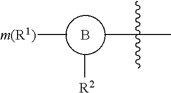
is

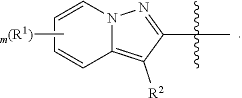

7. The compound of claim 1, wherein m is 1.

8. The compound of claim 1, wherein m is 2.

9. (canceled)

10. (canceled)

11. The compound of claim 1, wherein each R¹ is fluoro.

12.-15. (canceled)

16. The compound of claim 1, wherein m is 0.

17. (canceled)

18. (canceled)

19. The compound of claim 1, wherein R² is methyl.

20.-25. (canceled)

26. The compound of claim 1, wherein R³ is a C1-C6 haloalkyl.

27. The compound of claim 1, wherein R³ is difluoromethyl.

28. The compound of claim 1, wherein R³ is trifluoromethyl.

29. The compound of claim 1, wherein R³ is a C1-C6 alkyl.

30. The compound of claim 1, wherein R³ is Me, Et, or iPr.

31.-53. (canceled)

54. The compound of claim 1, wherein 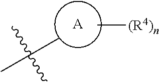
is 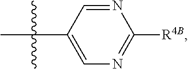
and R^{4B} is amino.

55.-63. (canceled)

64. A compound selected from the group consisting of 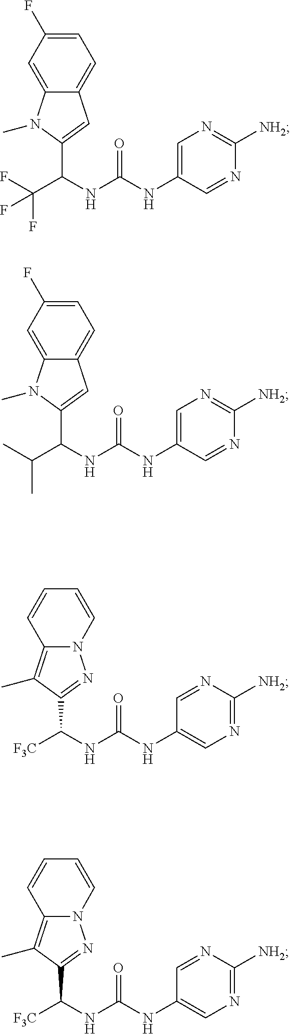 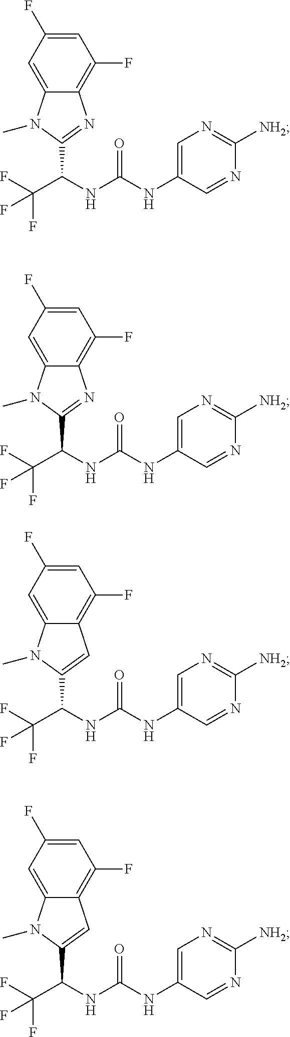 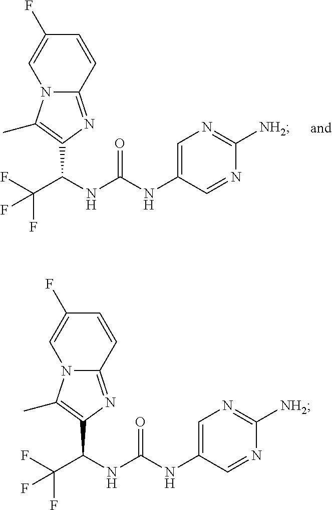 [SMILES: Cc1c([C@H](NC(=O)Nc2cnc(N)nc2)C(F)(F)F)nc2ccc(F)cn12 ; Cc1c([C@@H](NC(=O)Nc2cnc(N)nc2)C(F)(F)F)nc2ccc(F)cn12]
or a pharmaceutically acceptable salt thereof.

65. A pharmaceutical composition comprising a compound of claim 1, or a pharmaceutically acceptable salt thereof, and one or more pharmaceutically acceptable excipients.

66. A method for treating cancer in a subject in need thereof, the method comprising administering to the subject a therapeutically effective amount of a compound of claim 1, or a pharmaceutically acceptable salt thereof.

67. A method for treating cancer in a subject in need thereof, the method comprising (a) determining that the cancer is associated with a dysregulation of a PIK3CA gene, a PI3Kα protein, or expression or activity or level of any of the same; and (b) administering to the subject a therapeutically effective amount of a compound of claim 1, or a pharmaceutically acceptable salt thereof.

68. A method of treating a PI3Kα-associated cancer in a subject, the method comprising administering to a subject identified or diagnosed as having a PI3Kα-associated cancer a therapeutically effective amount of a compound of claim 1 or a pharmaceutically acceptable salt thereof.

69. A method for modulating PI3Kα in a mammalian cell, the method comprising contacting the mammalian cell with an effective amount of a compound of claim 1, or a pharmaceutically acceptable salt thereof.


SEQUENCE LISTING (1 sequence)

SEQ ID NO: 1 - protein, 1068 aa, Homo sapiens
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN